(19) United States
(12) Patent Application Publication
(10) Pub. No.: US 2004/0241720 A1
(43) Pub. Date: Dec. 2, 2004

(54) Sodium activated potassium ion channel for therapeutic drug screening

(76) Inventors: Lawrence B. Salkoff, Richmond Hts, MO (US); Alex Yuan, Saint Louis, MO (US)

Correspondence Address: Patrick W. Rasche, Armstrong Teasdale LLP, Suite 2600, One Metropolitan Square, St. Louis, MO, 63102, US

(21) Appl. No.: 10/787,726

(22) Filed: Feb. 26, 2004

Related U.S. Application Data
(60) Provisional application No. 60/450,530, filed on Feb. 26, 2003.
(60) Provisional application No. 60/451,181, filed on Feb. 28, 2003.

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

Isolated nucleic acid and amino acid sequences of the gene hSlo2.2, a Na+ sensitive potassium channel expressed in cardiomyocytes and neurons; antibodies to hSlo2.2; methods of screening for hSlo2.2 inhibitors; and methods of screening for hSlo2.2 homologues.

### CROSS REFERENCE TO RELATED APPLICATIONS

This application claims the individual and collective benefit of pending U.S. provisional patent application U.S. Ser. No. 60/450,530 filed Feb. 26, 2003 and pending U.S. provisional application U.S. Ser. No. 60/451,181 filed Feb. 28, 2003, both pending, each of which is incorporated herein in its respective entirety by reference.

### STATEMENT REGARDING FEDERALLY SPONSORED RESEARCH OR DEVELOPMENT

This invention was made with United States Government Support awarded by the National Institute of Health contract R01-DK54637. The United States Government has certain rights in this invention.

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

This invention generally relates to genes encoding and expressing proteins relating to ion channels, and individual proteins encoded and expressed thereby. Further, this invention relates to a method for identifying molecules that modulate and target sodium activated potassium channels.

### BACKGROUND OF THE INVENTION

Ion channels are membrane bound proteins that selectively permit the flow of ions across the membranes of cells. Illustrative ions include sodium, potassium, calcium and chloride. Ion channels are present in all mammalian cells, and play a crucial role in the operation of physiological and pharmacological processes within eukaryotic cells, including those of humans and primates.

Voltage gated sodium activated potassium channels, also referred to as sodium activated potassium channels, are found in neuronal, muscular, glandular, immune and epithelial tissue, and are involved in physiological processes, including regulation of heartbeat, dilation of arteries, release of insulin, excitability of nerve cells, and regulation of renal electrolyte transport. Further, these channels are critical in propagating the action potential in excitable cells including neurons and muscle cells as these ion channels regulate electrical potentials across cell membranes and the concentration of potassium within such cells.

Several mechanisms exist in cells for opening and closing of potassium ion channels, e.g., sodium concentration sensitivity, voltage-gating, and ATP-sensitivity. The regulation of potassium ion channels opening and closing is important for a number of cellular functions, e.g., controlling resting membrane potentials, providing feedback mechanisms for a train of action potentials, acting as a delayed rectifier in non-pathological states and assisting with preservation of cellular function under ischemic conditions. During an action potential, outward current carried by some potassium channels repolarizes the cell. Sodium/potassium-ATPase pumps are important in that these mechanisms control the concentration of intracellular potassium and extracellular sodium particularly in instances following repeated depolarization of the cellular membrane. In such situations the sodium/potassium-ATPase intracellular mechanism pumps sodium out of the cell and potassium into the cell, in effect maintaining the ambient concentrations of sodium and potassium ions inside the cell over time. In contrast, current through potassium channels is responsible for resetting the cell to propagate another action potential.

Other types of potassium channels are responsible for the controlling the resting potential, or intentionally lowering the excitability of the cell during conditions of cellular damage or stress. In this latter category, sodium activated potassium channels play an essential role, with native sodium activated potassium channels playing an important role under ischemic conditions and digitalis toxicity in cardiomyocytes (Kameyama et al., 1984; Luk and Carmeliet, 1990; Mitani and Shattock, 1992). Ischemia causes an increase in the concentration of sodium ions due primarily to the inhibition of the Na⁺/K⁺-ATPase pumps. The resulting activation of the sodium activated potassium channels reduces membrane electrical activity, improves calcium ion transport by preventing intracellular calcium ion accumulation, and may help protect the cells by preventing further excitable responses, action potentials; and, thus, conserve resources under ischemic conditions. In neurons, sodium activated potassium channels play a similar role (Haddad and Jiang, 1993; Dryer, 1994). In cardiomyocytes, sodium activated potassium channels also are involved in action potential shortening during ischemia (Dryer, 1994). Since sodium ion concentration is elevated in ischemic cardiomyocytes, sodium activated potassium channels contribute to action potential shortening.

The sequence of the rSlo2 gene (“rSlo2”) is reported and is publicly available in the public database (NCBI) under the sequence ID numbers: XM_{—}222699 Rattus norvegicus(Slack) [Rattus norvegicus] (LOC 304827), mRNA. gi/27712339/ref/xm_{—}222699.1/[27712339]. This gene was reported as the mammalian orthologue of the Slo-2 gene in C. elegans (“Slo-2”). Two mammalian Slo gene paralogues had also been characterized: Slo1 which encodes the BK channel, a Ca⁺ activated large conductance potassium ion channel (Atkinson et al., 1991; Adelman et al., 1992; Butler et al., 1993), and Slo3 which encodes a pH-sensitive potassium ion channel (Schreiber et al., 1998). Slo channels somewhat resemble voltage-gated potassium ion channels with the addition of a cytoplasmic carboxyl-terminal domain. Regions in the carboxyl-terminal domain like the RCK domain (Jiang et al., 2001; Jiang et al., 2002; Xia et al., 2002) and the calcium bowl (Schreiber and Salkoff, 1997; Screiber et al., 1999) are important for determining ionic sensitivity.

Because of the critical role played by sodium activated potassium channels in cellular function, it is highly desired to identify those genes encoding the protein(s) that make up sodium activated potassium ion channels.

### BRIEF DESCRIPTION OF THE INVENTION

For the first time isolated nucleotide and amino acid sequences of the human orthologue of the rSlo2 gene have been identified (“hSlo2.2”), are characterized and are provided herein. hSlo2.2 is a human gene which encodes a protein which is a voltage-regulated sodium activated potassium ion channel having novel functional properties and utilities, abundantly expressed in cardiomyocytes, neurons, and other tissues in the human body. The ion channel encoded by the hSlo2.2 gene exhibits sensitivity to intracellular concentrations of sodium (”[Na⁺]ᵢ”), and exhibits marked selectivity for sodium ions (“Na⁺ᵢ.”).

In addition to activation by [Na⁺]ᵢ, hSlo2.2 sodium activated potassium channels are cooperatively activated by intracellular chloride ions (“Cl⁻ᵢ”), and display cooperative sensitivity to the intracellular concentration of chloride ions (”Cl⁻]ᵢ”). Since intracellular [Na⁺]ᵢ and [Cl⁻]ᵢ both rise in oxygen-deprived cells, co-activation by both ions more effectively triggers the activity of hSlo2.2 channels in ischemic cells.

The present invention identifies and provides functional, isolated, and characterized nucleotide sequences of the novel human gene hSlo2.2, a sodium activated sensitive potassium channel expressed in cardiomyocytes and neurons; antibodies to hSlo2.2; amino acid sequences of hSlo2.2; methods of screening for hSlo2.2 inhibitors and activators; methods of identifying hSlo2.2 homologues; and methods of identifying agonists and antagonists specific for sodium activated potassium channels (“K_{Na}”).

In a first aspect, an isolated nucleic acid having nucleic acid sequence comprising a sequence of polynucleotides as shown in one of SEQ ID NO:1 or SEQ ID NO:2 or the complement thereof comprises a nucleic acid sequence which encodes a polypeptide monomer having the amino acid sequence SEQ ID NO:3, which encodes a sodium activated potassium channel wherein the peptide monomer (i) has a calculated molecular weight of about 120 kDa and about 150 kDa; (ii) has a unit conductance of about 60 to 190 pS, which depends upon the ionic conditions of measurement when the monomer is in a functional tetrameric form of a potassium channel and is expressed in a Xenopus oocyte or mammalian cell; (iii) has increased activity when intracellular sodium concentrations are raised from 1 to 150 mM; and (iv) specifically binds to polyclonal antibodies generated against SEQ ID NO:3, and forms a sodium activated potassium channel. In an aspect, the nucleic acid encodes and expresses hSlo2.2. In an aspect hSlo2.2 has a sequence of at least one of SEQ ID NO:1 and SEQ ID NO:2.

In an aspect, the nucleic acid has a nucleotide sequence of at least one of SEQ ID NO:1 and SEQ ID NO:2 encodes the amino acid sequence SEQ ID NO:3 having ion channel activity.

In an aspect, the nucleic acid selectively hybridizes under moderate stringency hybridization conditions to at least one of SEQ ID NO:1 and SEQ ID NO:2.

In an aspect, the nucleic acid having a nucleotide sequence of at least one of SEQ ID NO:1 and SEQ ID NO:2 encodes the amino acid sequence SEQ ID NO:3 having ion channel activity.

In an aspect, the nucleic acid having a nucleotide sequence of at least one of SEQ ID NO:1 and SEQ ID NO:2 is amplified by primers that selectively hybridize under stringent hybridization conditions to the same sequence as the primer sets selected from the group consisting of:

| | | |
|---|---|---|
| Set 1: | | |
| Upper: | GCGGGCGGGCGAGAACCTGTC | (SEQ ID NO:4) |
| Lower: | GTAGAGGGGGCAGTTGGGGGCGAAGT | (SEQ ID NO:5) |
| Set 2: | | |
| Upper: | ACTTCGCCCCCAACTGCCCCCTCTAC | (SEQ ID NO:6) |
| Lower: | GTCCTCCCGCTTCAGCCCGATGAG | (SEQ ID NO:7) |
| and | | |
| Set 3: | | |
| Upper: | GGGCTGAAGCGGGAGGACAACAAGAG | (SEQ ID NO:8) |
| Lower: | GCTTTCACAGGCAGGAGGTGGCACAG | (SEQ ID NO:9) |

and the nucleic acid having the nucleotide sequence SEQ ID NO:2 is amplified by primers that selectively hybridize under stringent hybridization conditions to the same sequence as the primer set consisting of: such primer sets, set 1, 2, 3 and 4 hybridizing to regions of the nucleotide sequence of hSlo2.2 that are conserved across all slice variants of the gene.

| | | |
|---|---|---|
| Set 4: | | |
| Upper: | ATGAGCGACCTGGACTCCGAGGTGCTG | (SEQ ID NO:10) |
| Lower: | TCAGAGCTGTGTCTCGTCGCGAGTCTC | (SEQ ID NO:11) |

In an aspect, an isolated nucleic acid encodes and expresses at about 1151 to about 1500 contiguous amino acids forming a sodium activated potassium channel polypeptide monomer, said monomer having an amino acid sequence of SEQ ID NO:3, and conservatively modified variants thereof.

In an aspect, the monomer comprises 1204 amino acids and has a molecular weight of 136612.70. In an aspect hSlo2.2 is expressed in humans.

In an aspect, an isolated nucleic acid encodes a sodium sensitive potassium channel polypeptide monomer having: (i) unit conductance of about 60 pS to about 180 pS when the monomer is in a functional tetrameric form of a potassium channel and is expressed in a heterologous cell system such as Xenopus oocytes or a mammalian cell line; (ii) a molecular weight about 120 kDa to about 150 kDa; and (iii) increased activity above an intracellular sodium concentration of about 1 mM; and where the nucleic acid either: (i) selectively hybridizes under moderate stringency hybridization conditions to a nucleotide sequence comprising at least one of SEQ ID NO:1 and SEQ ID NO:2 or (ii) encodes a protein which can be encoded by a nucleic acid that selectively hybridizes under moderate stringency hybridization conditions to a nucleotide of SEQ ID NO:3.

In an aspect, an isolated nucleic acid encodes a polypeptide monomer of a sodium activated potassium ion channel, the sequence: (i) encoding a monomer having a core domain that has greater than 60% amino acid sequence identity to amino acids 200-600 of a hSlo2.2 core domain as measured using a sequence comparison algorithm; and (ii) specifically binding to polyclonal antibodies raised against the core domain of SEQ ID NO:3.

In an aspect, the invention provides an isolated polypeptide monomer of a sodium sensitive potassium channel, the monomer having: (i) a calculated molecular weight of about 120 to about 50 kDa; (ii) a unit conductance of about 60 to about 80 pS when the monomer is in a functional tetrameric form of a potassium channel and is expressed in a cell; (iii) increased activity above approximately intracellular sodium concentration of about 1 mm; and (iv) specifically binding to polyclonal antibodies generated against at least one of SEQ ID NO:1 and SEQ ID NO:2.

In an aspect, the invention provides an antibody that selectively binds to hSlo2.2.

In an aspect, an expression vector comprises a nucleic acid encoding a polypeptide monomer of a sodium activated potassium channel, the monomer having: (i) a calculated molecular weight of between about 120 kDa to about 150 kDa; (ii) a unit conductance of about 60 to about 80 pS when the monomer is in a functional tetrameric form of a potassium channel and is expressed in a cell; (iii) increased activity above approximately intracellular sodium concentration of about 1 mm; and (iv) specifically binding to polyclonal antibodies generated against at least one of SEQ ID NO:1 and SEQ ID NO:2.

In an aspect, a host cell is provided having the expression vector.

In an aspect, a method for identifying a compound that modulates ion flux through a sodium activated potassium channel encoded by a gene selected from at least one of hSlo2.2 or rSlo2 comprises: (i) contacting one of a eukaryotic host cell or cell membrane in which has been expressed a sodium sensitive potassium channel monomer polypeptide with a candidate compound: (a) the peptide having a calculated molecular weight of between 120 kDa and 150 kDa; (b) having a unit conductance of about 60 pS to about 180 pS when the monomer is in the functional tetrameric form of a potassium channel and is expressed in an eukaryotic cell; and (c) specifically binding to polyclonal antibodies generated against at least one of SEQ ID NO:1 and SEQ ID NO:2; and (ii) determining the functional effect of the compound upon the cell or cell membrane expressing the sodium activated potassium channel.

In an aspect, the modulation flux of ions is determined by measuring whole cell conductance. In another aspect, modulation flux is selected from increasing flux, steady flux, changing flux and decreasing flux.

In an aspect, the sodium activated potassium channel monomer polypeptide is recombinant.

In an aspect, a method of detecting the presence of hSlo2.2 in mammalian tissue comprises (i) obtaining and isolating a functional biological sample from a patient; (ii) contacting the biological sample with a hSlo2.2 specific reagent that selectively binds to hSlo2.2 and, (iii) detecting the level of hSlo2.2 specific reagent that selectively associates with the functional biological sample.

In an aspect, the hSlo2.2 specific reagent is selected from the group consisting of hSlo2.2 specific antibodies, hSlo2.2 specific oligonucleotide primers and Slo2 nucleic acid probes. In an aspect, the biological sample is from a human.

In an aspect, the invention provides in a computer system, a method of screening for mutations of hSlo2.2 genes, comprising: (i) receiving input of a first nucleic acid sequence encoding a sodium activated potassium channel protein having a nucleotide sequence of at least one of SEQ ID NO:1 and SEQ ID NO:2, and conservatively modified versions thereof; (ii) comparing the first nucleic acid sequence with a second nucleic acid sequence having substantial identity to the first nucleic acid sequence; and (iii) identifying nucleotide differences between the first nucleic acid sequence and its second nucleic acid sequence.

In an aspect, the invention provides in a computer system, a method for identifying a three-dimensional structure of hSlo2.2 proteins, the method comprising: (i) receiving input of about 270 to 900 nucleotides or 90 to 300 amino acids of an amino acid sequence of a sodium activated potassium channel monomer or a nucleotide sequence of a gene encoding the protein, the protein having an amino acid sequence of at least one of SEQ ID NO:1 and SEQ ID NO:2, and conservatively modified versions thereof; and (ii) generating a three-dimensional structure of the protein encoded by the amino acid sequence.

In an aspect, the amino acid sequence is a primary structure generated by a process comprising (i) forming a secondary structure from said primary structure using energy terms encoded by the primary structure and (ii) forming a tertiary structure from the secondary structure using energy terms encoded by the secondary structure.

In an aspect, the generation includes forming a quaternary structure from the tertiary structure using anisotrophy terms encoded by the tertiary structure.

In an aspect, the generation further comprises identifying regions of the three-dimensional structure of the protein that bind to ligands and using the regions to identify ligands that bind to the protein.

In an aspect, a transgenic cell is provided, the transgenic cell having a gene comprising an isolated nucleic acid selected from one of hSlo2.2 or rSlo2 stably integrated in its germane encoding (and expressing) a polypeptide monomer of sodium activated potassium channel(s), and having an assay system operably connected thereto, wherein the transgenic cell and associated assay system is configurably enabled to produce an output associatively indicative of sodium activated potassium ion channel activity modulated by presented molecule (compound). In an aspect, the compound is externally presented.

In an aspect, a biosensor and comprising a transgenic cell having stably integrated in its genome a gene selected from at least one of hSlo2.2 and rSlo2 which encodes a respective protein which functions as a cloned sodium activated potassium ion channel, the transgenic cell having an assay system operatively coupled thereto wherein the assay system is configurably enabled to produce an output signal associatively indicative of sodium induced potassium ion channel modulation.

In an aspect, a candidate compound is added to the biosensor and the assay system is used to classify the compound as an agonist, antagonist or ineffective compound.

In an aspect, a method of preparing a biosensor comprises competently and stably transfecting a gene selected from one of hSlo2.2 or rSlo2 having a nucleic acid functionally encoding a sodium activated potassium ion channel as a stable expression product in a cell, coupling an assay operatively to the cell, configuring the assay to capably produce an output associatively indicative of the activity of the channel encoded and expressed by the hSlo2.2 or rSlo2 gene in the biosensor. In an aspect, the biosensor and assay system are suitably operably coupled and configured so as to provide an assay system wherein multiple additions are made to one or more transgenic cells so that one or more assay send outs are obtained, thus providing a high through out screening system for therapeutic drug screening.

In an aspect, method for determining the expression of the genes hSlo2.2 or rSlo2 comprises using antibodies in vitro as diagnostic tools to examine hSlo2.2 or rSlo2 gene expression.

In an aspect, a method for treating a patient having conditions related to cardiomyocyte and neuron physiology including ischemic conditions and digitalis toxicity comprises isolating cells from the patient, transfecting the isolated cells with a hSlo2.2 channel protein nucleic acid (gene or cDNA), and re-infusing the transfected cells back into the patient. In an aspect the patent is a known patent.

In an aspect, a method for preconditioning organ transplants comprises isolating cells from the candidate organ explant (donor), transfecting the candidate explant with a hSlo2.2 channel protein nucleic acid (gene or cDNA) and re-infusing the transfected cells back into the candidate organ explant prior to harvesting of the organ from the donor.

In an aspect, a method for preconditioning organ transplants comprises transfecting the candidate explant organ with a hSlo2.2 nucleic acid (gene or cDNA).

In an aspect, a kit for determining in a biological sample the presence or absence of a normal gene selected from one of hSlo2.2 or rSlo2 or gene product encoding one of hSlo2.2 or rSlo2 encoded protein, for the presence or absence of an abnormal gene or gene product encoding a hSlo2.2 or rSlo2 sodium activated potassium channel, or quantification of the transcription levels of normal or abnormal hSlo2.2 or rSlo2 channel protein gene product, the kit comprising a stable preparation of nucleic acid probes for performing the assay of the present invention and optionally includes a hybridization solution in either dry or liquid form for the hybridization of probes to target sodium sensitive potassium channel proteins or sodium sensitive potassium channel protein nucleic acids of the present invention, a solution for washing and removing undesirable and non-hybridized nucleic acids and a substrate suitable for detecting the hybridization complex.

In an aspect, a method is provided for modulating the membrane potential of a cell which comprises adding a voltage modulating effective amount of an agonist to a transgenic cell expressing a gene selected from one of hSlo2.2 and rSlo2 whereby the cell membrane potential is modulated to place the cell in a position of increased or maximum potassium ion outflow. The effective amount of agonist is that amount which opens and renders active a sodium activated potassium ion channel yet is a nontoxic, transgenic cell life sustaining amount.

In an aspect, a method of treating a central or peripheral nervous system disorder or condition of a subject through modulation of a voltage-dependent potassium channel comprises administering to a subject in need of such treatment, an effective amount of a compound which increases the sodium activated potassium channel activity.

In an aspect, a method for reducing pain in a subject in need thereof by increasing ion flow through K potassium channels in a cell comprises administering to the subject a pharmaceutical composition comprising a pharmaceutically acceptable carrier and a compound able to increase ion flow through sodium activated potassium channels, when the composition administered to the subject is an effective potassium channel opening amount, thereby reducing pain in the subject.

In an aspect, a nucleic acid molecule comprises a nucleotide sequence which encodes protein comprising SEQ ID NO:3 or the complement thereof, selected from (a) a nucleic acid molecule comprising a nucleotide sequence as shown in SEQ ID NO:1 or SEQ ID NO:2 or the complement thereof as a nucleic acid molecule which is capable of hybridization under high stringency conditions to a nucleic acid molecule as set forth in a) above.

In an aspect hSlo2.2 comprises cDNA's regardless of length which hybridize under stringent conditions to nucleic acids SEQ ID NO:1 or SEQ ID NO:2.

In an aspect hSlo2.2 comprises cDNA's regardless of length which hybridize under moderate conditions to nucleic acids SEQ ID NO:1 or SEQ ID NO:2, and which hybridize under stringent conditions to human chromosome 9q34.

### BRIEF DESCRIPTION OF THE DRAWINGS

FIG. 1 depicts overall structural features of hSlo2.2 channels. hSlo2.2 channels are members of the Slo gene family of large and intermediate conductance K⁺ channels activated by intracellular ions.

FIG. 1a is a schematic representation comparing the structures of voltage-gated K⁺ channels (top), Slo-1 Ca²⁺ and voltage activated channels (middle), and Slo-2 channels (bottom). Both Slo-1 and Slo-2 channels resemble voltage-gated K⁺ channels but also contain a long cytoplasmic carboxy-terminus domain. In Slo-1 channels, the “calcium bowl” (Schreiber and Salkoff, 1997; Schreiber et al., 1999) and two predicted RCK domains (Jiang et al., 2002; Xia et al., 2002) are important for sensing intracellular Ca²⁺. Slo-1 channels also include an additional S0 transmembrane domain (Wallner et al., 1996; Meera et al., 1997). In place of the “calcium bowl”, Slo-2 channels contain a “chloride bowl” (Yuan et al., 2000). Slo-2 channels are absent an S0 transmembrane domain.

FIG. 1b depicts a sequence alignment in the “chloride bowl” of Slo-2 channels, and the analogous region of the “calcium bowl” of Slo-1 channels. Black shaded residues are identical in all Slo channels. Grey shaded residues are identical in all Slo-2 channels. Three positions (boxed) in Slo-2 channels are notably different between C. elegans Slo-2 (nSlo2.2) and mammalian Slo-2 (rSlo2.2 and mSlo2.2). Locations of the “calcium bowl” and “chloride bowl” are shown in Yuan et al., 2000.

FIG. 2 depicts rSlo-2 as activated by sodium. We investigated the properties of rSlo 2 channels in the Xenopus oocyte expression system by transcribing cRNA and injecting it into oocytes; the single electrode patch clamp system was used to analyze inside-out patches.

FIG. 2a depicts current traces from an inside-out macropatch (2-3 MΩ electrode prior to seal formation) in the presence of 50 mM [Na+], FIG. 2a (i) and in the absence of [Na⁺]ᵢ FIG. 2a (ii). Voltage steps (−70 mV to +70 mV) were applied in 10 mV increments from a holding potential of −70 mV. The calculated potassium equilibrium potential was −78 mV in these experiments. FIG. 2a (iii) The current-voltage relationship of the mean current obtained from 7 macropatches with a holding potential of −80 mV, and voltage steps from −80 to +80 mV in 10 mV increments FIG. 2a (iv) Plot of normalized IK_{Na} obtained upon perfusion of inside-out patches with different Na⁺ concentrations as indicated.

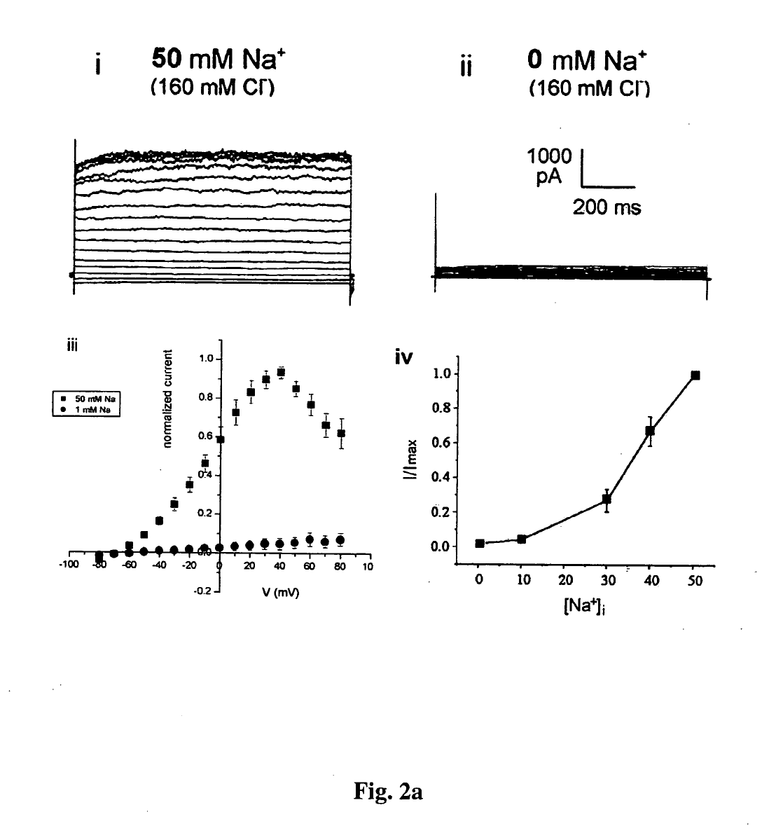

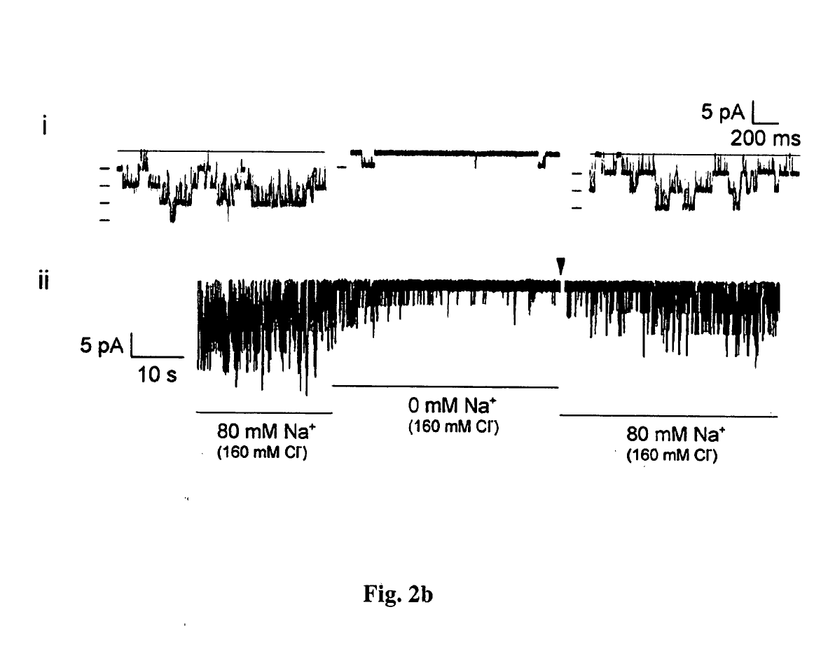

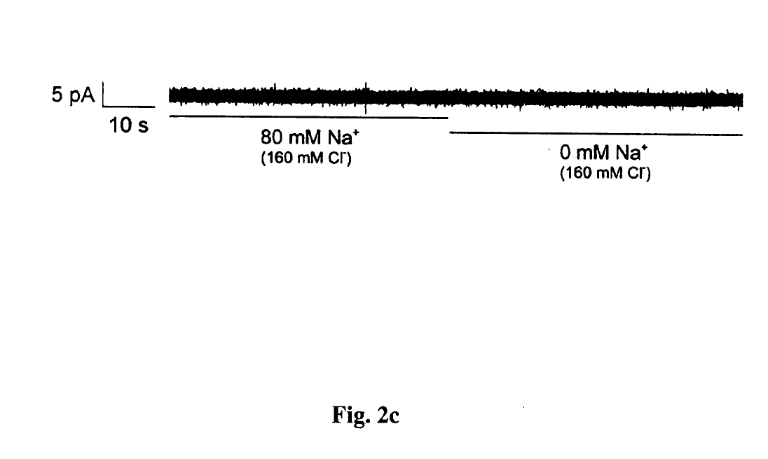

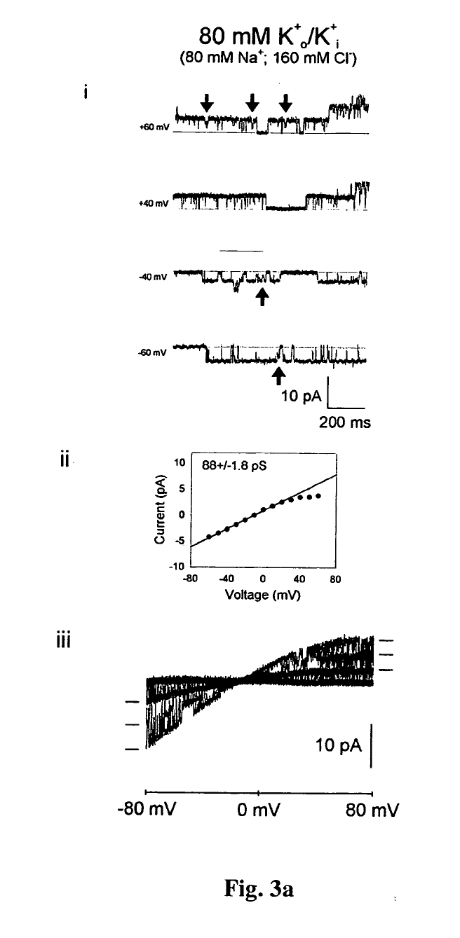

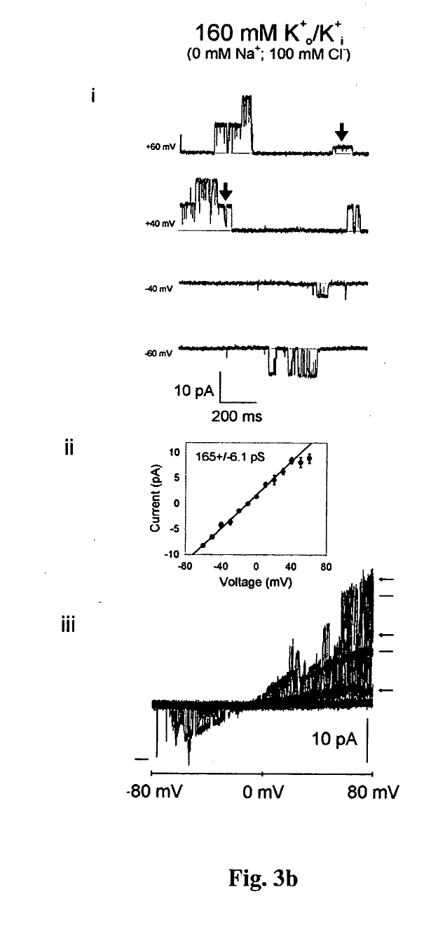

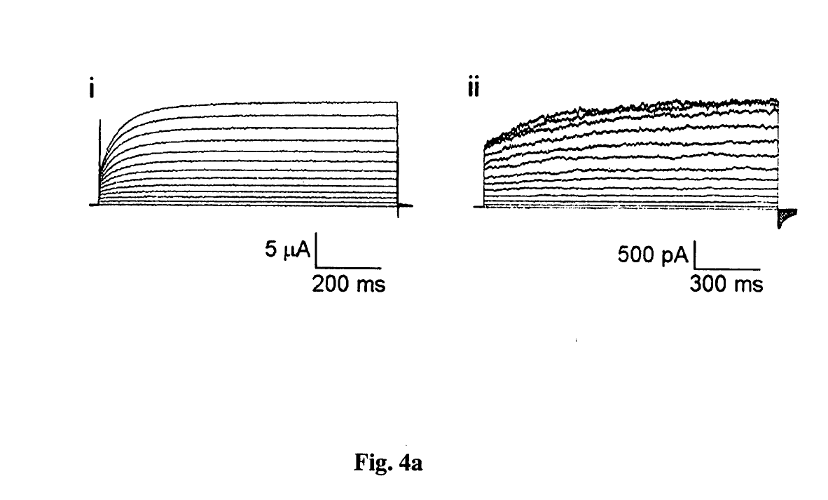

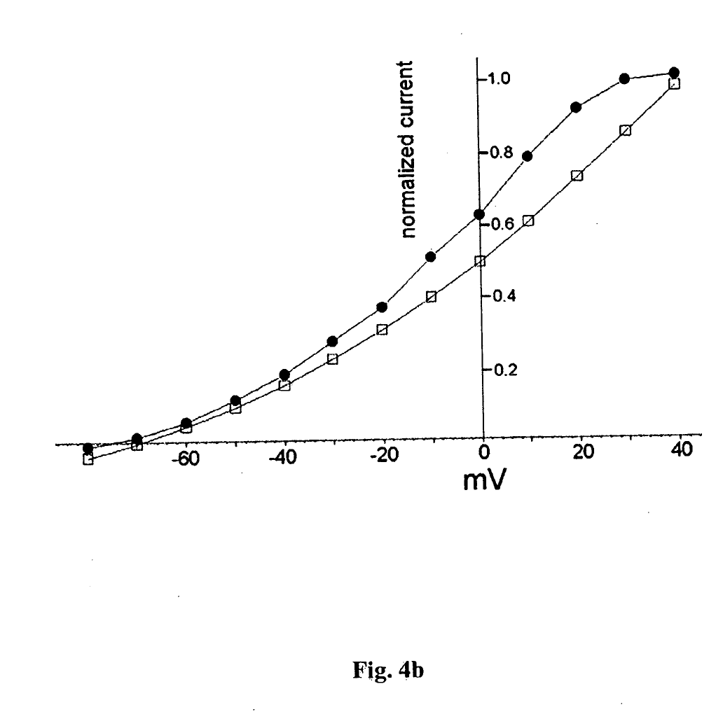

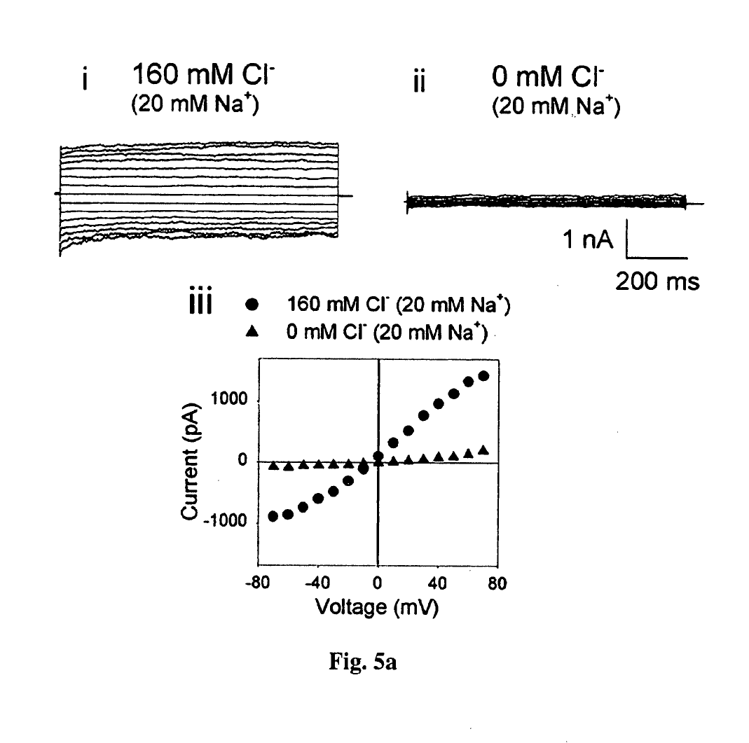

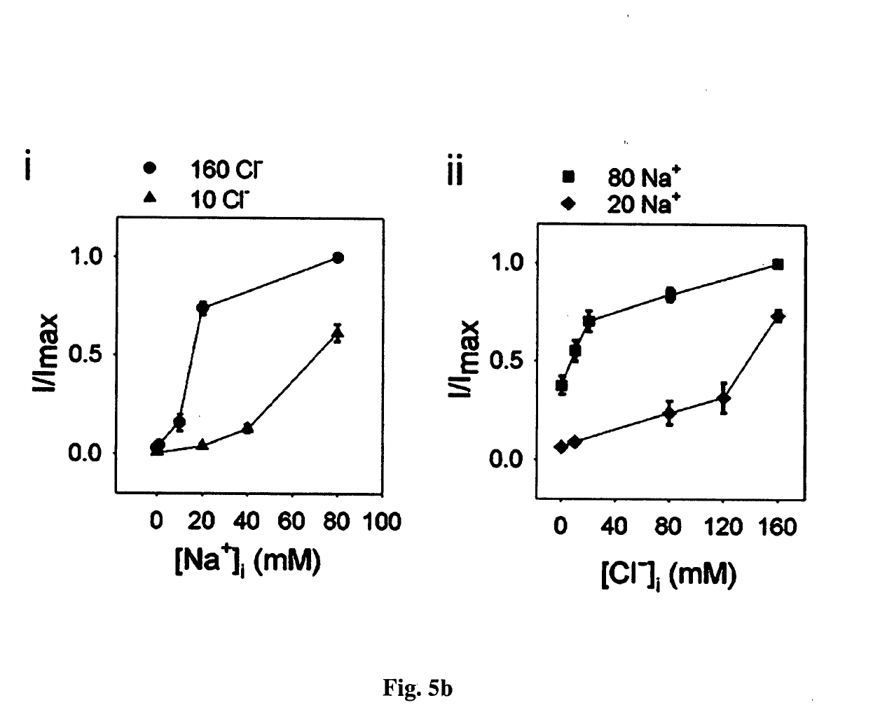

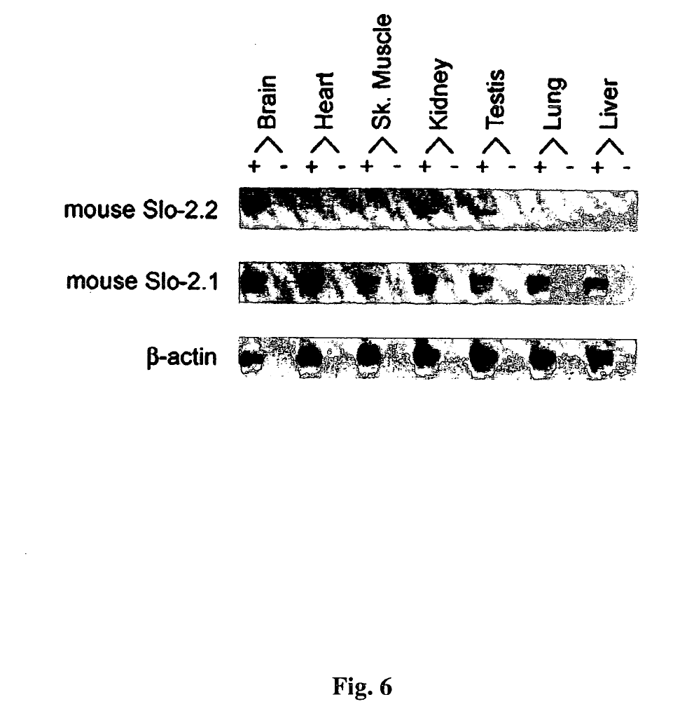

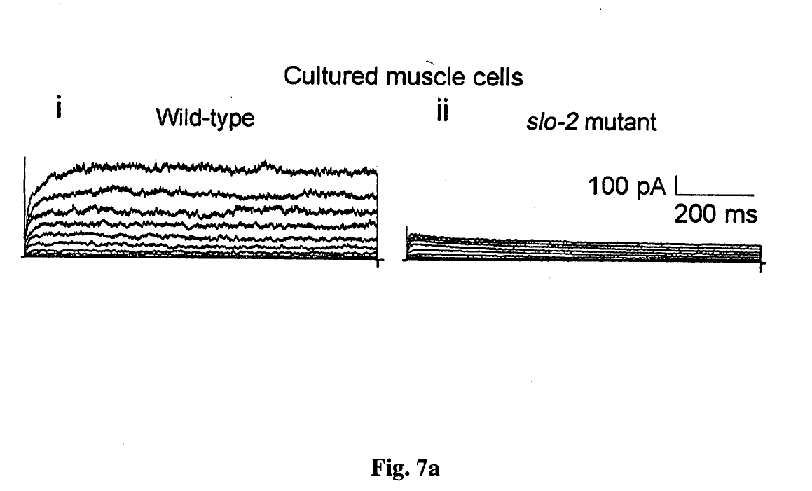

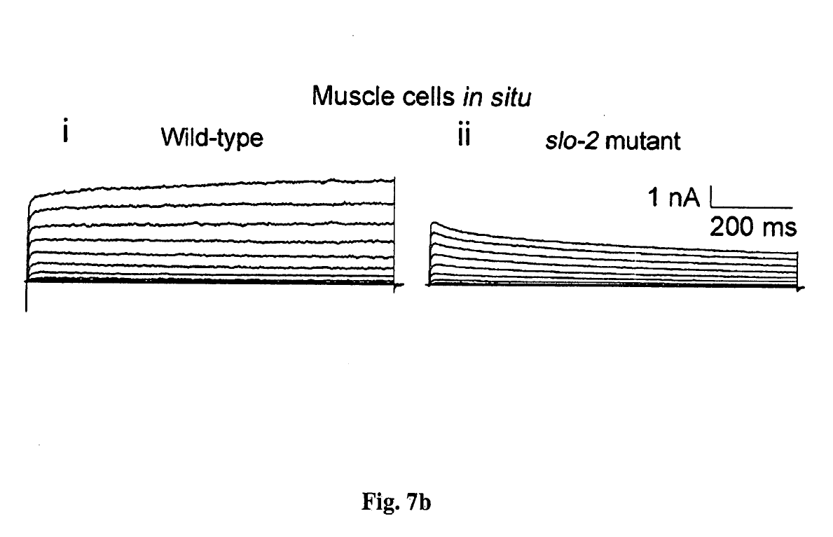

Further, FIG. 2a i depicts macroscopic currents from an inside-out patch. In the presence of sytoplasmic Na⁺, rSlo-2 channels produced large (multi nanoamp) K⁺ currents that showed slight outward rectification over a wide voltage range (−70 mV to +30 mV). No macroscopic channel activity was detected in the absence of [Na⁺]ᵢ(FIG. 2a (ii)). Although the current-voltage relationship for rSlo-2 shows slight outward rectification between −70 mV to +30 mV, “inward rectification” is seen at potentials more positive that +30 (FIG. 2a (iii)). This phenomenon has been observed in native K_{Na} and may be due to Na⁺ block of the outward K⁺ current (Wang et al., 1991; Egan et al., 1992b). The actrivation of macroscopic currents in patches is sharply dependent on the concentration of sodium ion that the cytoplasmic side of the patch is exposed to, with the amplitude of current increasing more than ten times as the sodium concentration is increased from 10 to 50 mM (FIG. 2a (iv)).

FIG. 2b depicts single channel activity over many seconds; activity dramatically decreased when Na⁺ᵢ was removed, but increased upon reintroduction of high Na⁺ᵢ. Like K_{Na} channels from native tissue, rSlo-2 channels showed rundown. Channel activity decreased with time, possible suggesting the wash-out of an intracellular factor required to maintain full channel activity. Single channel currents from an inside-out patch (10 MΩ electrode prior to seal formation) held at −60 mV and perfused with 80 mM [Na⁺]ᵢ or 0 mM [Na⁺]ᵢ shown on an expanded time scale FIG. 2b (i) and a compressed time scale in FIG. 2b (ii). Four unitary conductance levels can be seen. The arrowhead indicates a gap of ˜50 s of data not shown. Symmetric (80 mM) K⁺ was used in these tests.

FIG. 2c depicts a current trace of an uninjected oocyte. Although a previous report indicated that endogenous K_{Na} were sparsely present in the membrane of Xenopus oocytes (Egan et al., 1992a), recordings from inside-out patches of uninjected oocytes under the conditions we used did not reveal any Na⁺-activated K⁺ channels (n=10). On the other hand, most (>90%) of the patches from oocytes injected with hSlo2.2 cRNA produced multiple channels in high [Na⁺]ᵢ, and macroscopic currents were observed in a majority of macropatches 3-5 days post injection. For the macropatch experiments the pipette solution contained (in mM) 145 Na⁺-gluconate, 5 KCI, 5 Ca²⁺-gluconate, 3 Mg²⁺-gluconate, 11 dextrose, 5 HEPES, pH 7.4 with NaOH. The bath solution contained (in mM): 110 KCI, 50 NaCI and 5 HEPES, pH 7.3 with KOH. NaCI was replaced by choline-Cl for the dose-response curve. In single channel experiments, the pipette solution contained (in mM) 80 K⁺-gluconate, 80, 5 HEPES, 2 MgCI₂, pH 7.2 with KOH. The bath solution contained (in mM) 80 KCI, 80 NaCI or 80 cholineCl, 5 HEPES, 5 EGTA, pH 7.2 with KOH.

FIG. 3 depicts single rSlo-2 channels. Single channel conductance of rSlo-2 is dependent on K⁺₀ and K⁺ᵢ. (a) Single channels recorded from an inside-out patch in 80 mM symmetrical K⁺₀/K⁺ᵢ.

FIG. 3a (i) depicts currents from a patch held at +60, +40, −40, and −60 mV. Arrows indicate subconductance states.

FIG. 3a (ii) depicts mean current-voltage relationship of single channels from multiple patches (n=5). The slope conductance, measured from −60 mV to +10 mV, is 88+/−1.8 pS.

FIG. 3a (iii) depicts currents evoked by 12 superimposed voltage ramps from −80 mV to +80 mV. Three main conductance levels are present (horizontal lines). The pipette solution contained (in mM) 80 K⁺-gluconate, 80 Na⁺-gluconate, 5 HEPES, 2 MgCI₂, pH 7.2 with KOH. The bath solution contained (in mM) 80 KCI, 80 NaCI, 5 HEPES, 5 EGTA, pH 7.2 with KOH.

FIG. 3b depicts single channels recorded from inside-out patches in 160 mM symmetrical K⁺₀/K⁺ᵢ.

FIG. 3b (i) depicts currents from a patch held at +60, +40, −40, and −60 mV. Arrows in FIG. 3b(i) indicate subconductance states.

FIG. 3b (ii) depicts mean current-voltage relationship of single channels from multiple patches (n=3). The slope conductance, measured from −60 mV to +10 mV, is 165+/−6.1 pS.

FIG. 3b (iii) depicts currents evoked by 12 superimposed voltage ramps from −80 mV to +80 mV. Two main conductance levels are present (horizontal lines). Subconductance states of approximately 50 pS (at +80 mV) are indicated by arrows. The pipette solution contained (in mM) 156 KK⁺₀/K⁺-gluconate, 4 KCI, 10 HEPES, pH 7.3 with KOH. The bath solution contained (in mM) 100 KCI, 30 KK⁺₀/K⁺-gluconate, 10 HEPES, 11 EGTA, pH 7.2 with 30 KOH.

FIG. 4 depicts whole cell rSlo-2 currents from heterologous expression in Xenopus oocytes compared with currents from macropatches.

FIG. 4a (i) depicts current traces from a Xenopus oocyte injected with rSIo2 cRNA and analyzed with the two electrode voltage clamp method. Approximately 15 _{μ}A of outward current was present while uninjected control oocytes expressed less than 100 ηA of current. Voltage steps (−80 mV to +40 mv) were applied in 10 mV increments from a holding potential of −70 mV. Fitting the time-dependent rise of current at +80 mV with a single exponential produced a time constant of approximately 70 ms. Recording pipettes were filled with 3 M KCI and the oocyte bath solution contained (in mM) 96 NaCI, 2 KCI, 1.8 CaCl₂, 1 MgCI₂, 5 HEPES, pH 7.5 with NaOH.

FIG. 4a (ii) depicts current traces from an inside-out macropatch (2-3 MΩ electrode prior to seal formation) in the presence of 50 mM [Na+]1. Voltage steps (−80 mV to +40 mV) were applied in 10 mV increments from a holding potential of −70 mV. Solutions are similar to those used in FIG. 2a (i).

FIG. 4b depicts the normalized current-voltage relationship of the maximal current from the above traces. The currents reversed close to the potassium ion equilibrium potential (˜−78 mV in macropatch experiments).

FIG. 5 depicts rSlo-2 activated by chloride.

FIG. 5a depicts current traces from an inside-out macropatch (2 MΩ electrode tip) in the presence of 160 mM [CI]ᵢ (i) and in the absence of [CI]_{I}

FIG. 5a (ii) depicts voltage steps (−70 mV to +70 mv) were applied in 10 mV increments from a holding potential of 0 mV.

FIG. 5a (iii) depicts the current-voltage relationship of the mean current between 700-800 ms. The pipette solution contained (in mM) 80 K⁺-gluconate, 80 Na⁺-gluconate, 5 HEPES, 2 MgCI₂, pH 7.2 with KOH. The bath solution contained (in mM) 80 KCI or 80 K⁺-gluconate, 20 NaCI and 60 choline-Cl or 20 Na⁺-gluconate and 120 dextrose, 5 HEPES, 5 EGTA, pH 7.2 with KOH. (b) (i) Dose response relationship for [Na+]i at 160 mM [CI]ᵢ or 10 mM [CI]ᵢ,. (ii) Dose response relationship for [CI]ᵢ at 80 mM [Na⁺]ᵢ or 20 mM [Na⁺]ᵢ. Currents for the dose response curves were measured and averaged over a 1 second interval at −60 mV and each patch was normalized to the current elicited in 80 mM [Na⁺]ᵢ, and 160 mM [CI]ᵢ. Each experiment was completed within 120 seconds to minimize rundown and was perfused in order of decreasing [Na⁺]ᵢ or [CI]ᵢ.

FIG. 6 depicts the expression of two hSlo2.2 paralogues in different tissues from mouse. Results are from RT-PCR tests using primers specific for mouse Slo2.2, mouse Slo2.1, and a β-actin control. Note that Slo2.2 is the mouse orthologue of rSlo2. Sbo2.2 was detected in brain, kidney, heart, testis. A faint signal was also detected in heart. Sbo2.1 was detected in all tissue types tested. A plus sign indicates the addition of reverse transcriptase to the reaction. A minus sign indicates a control in the absence of reverse transcriptase.

FIG. 7 depicts Slo-2 current as the major component of the delayed-rectifier in C. elegans muscle.

FIG. 7a depicts current traces from whole-cell patch clamp recordings of wild-type and slo-2 mutant muscle cells in culture. (i) In most (15/22) wild-type cells, the delayed outward current is larger than the transient current. (ii) In all (15/15) slo-2(nf100) mutant cells, the transient current is the larger component. The average delayed current measured between 800-900 ms at +60 mV was 37.6+1−5 pA/pF (n=22) for wild-type cells, and 10.0+1−1 pA/pF (n=15) for mutant cells.

FIG. 7b depicts current traces from whole-cell patch clamp recordings of wild-type and slo-2 mutant body wall muscle cells recorded in situ from adult animals.

FIG. 7b (i) depicts in most wild-type cells (4/5) the delayed current is larger than the transient component.

FIG. 7b(ii) shows that in all (6/6) of the slo-2(nf100) mutant cells, the transient current is the larger component. The average delayed current measure between 800-900 ms at +60 mV was 130.1+/−21 pA/pF (n=5) for wild-type cells, and 48.8+/−10 pA/pF (n=6) for mutant cells. Voltage steps (−70 mV to +60 mV) were applied in 10 mV increments with a holding potential of −70 mV. We confirmed these results with two slo-2 deletion alleles: slo-2(nf100) and slo-2(nf101) (Wei et al, 2002). The pipette solution contained (in mM) 120 KCI, 20 KOH, 4 MgCI₂, 5 Tris, 0.25 CaCI₂, 36 sucrose, 5 EGTA, 4 Na₂ATP, pH 7.2 with HCI. The external solution contained (in mM) 140 NaCI, 5 KCI, 5 CaCI₂, 5 MgCI2, 11 dextrose, 5 HEPES, pH 7.2 with NaOH.

### DETAILED DESCRIPTION OF THE INVENTION

The present invention provides for the first time isolated nucleic acid and amino acid sequences for the human gene hSlo2.2 and demonstrates that hSlo2.2 encodes a sodium (“Na+”) activated potassium channel. The inventors discovered that functionally, the gene hSlo2.2 is expressed in cardiomyocytes and neurons, among other tissues, is voltage-gated, and is Na⁺ sensitive. Furthermore, the expressed product protein (SEQ ID NO:3) hSlo2.2 exhibits markedly greater Na⁺ selectivity and sensitivity than most voltage-gated potassium channels. hSlo2.2 mRNA was detected in cardiomyocytes and neurons, where the mRNA was abundantly expressed. This expression pattern and sensitivity to both Na+ and voltage, show that the gene hSlo2.2 is involved in activating potassium channel proteins in response to an increase in intracellular Na⁺ concentrations.

Structurally, the full length nucleic acid sequence of hSlo2.2 (SEQ ID NO:1) encodes (id expresses) a protein of about 1151 amino acids (SEQ ID NO:3). hSlo2.2 is a member of the Slo potassium channel protein family as evidenced by sequence homology to the BK calcium-activated potassium channel. Hydrophilicity profiles of the hSlo2.2 sequence indicate six hydrophobic segments, S1 through S6, which define the “core” domain. The amino acid sequences which follow the core domain are involved in sensing the concentrations of intracellular ions. (Wei et al., Neuron 13:671-681 (1994)) Yuan, A., Dourado, M., Butler, A., Walton, N., Wei, A., and Salkoff, L. (2000). Slo-2, a K⁺ channel with an unusual Cl-dependence, Nat Neurosci 3, 77 1-9.

This discovery demonstrates that hSlo2.2 sodium activated potassium channels are related to sodium activated potassium channels encoded by rSlack [Joiner, 1998 #9] (“rSlo2”), a gene originally cloned from Rattus norvegicus, and a member of the Slo family of potassium (“K⁺”) channels, other of which encode high and intermediate conductance channels sensitive to a variety of intracellular factors.

The single electrode patch clamp system was first used to analyze inside-out patches, and to investigate the properties of rSlo2 in the Xencpus oocyte expression system by transcribing cRNA and injecting it into oocytes.

Calcium Sensitivity of rSlo2 Channels

rSlo2 was previously reported as being inhibited by calcium ions (“Ca²⁺ᵢ”) [Joiner, 1998 #9]. In that study, rSlo2 channels were not exposed to sodium ions (“Na⁺ᵢ”); and, therefore, channel openings were infrequent. Confirmation of this observation in the absence of Na⁺ᵢ, where single channel activity was inhibited with about 100 μM Ca²⁺ᵢ (data not shown) was performed. However, the presence of about 80 μM ˜200 μM Ca²⁺ᵢ had no effect on channel activity (data not shown), which is consistent with reports that the native sodium activated potassium channels are insensitive to Ca²⁺ᵢ [Dryer, 1989 #3; Haimann, 1990 #21; Haimann, 1992 #13].

Single Channel Properties

Single channel properties of rSlo2 closely resembled those of native sodium activated potassium channels. The single channel conductance of native sodium activated potassium channels is strongly dependent on potassium ion concentration (”[K⁺]ᵢ”) [Dryer, 1989 #3; Haimann, 1990 #21; Safronov, 1996 #6; Mistry, 1997 #22]. In one example, native sodium activated potassium channels recorded from guinea pig cardiomyocytes had a conductance of 75 pS in symmetrical 60 mM K⁺ₒ/K⁺ᵢ, and 220 pS in 150/70 mM K⁺ₒ and K⁺ᵢ[Mistry, 1997 #22]. For rSlo2, when the potassium ion concentration was increased from symmetrical 80 mM K⁺ₒ and K⁺ᵢ to 160 mM K⁺ₒ and K⁺ᵢ, the slope conductance increased from 88+/−1.8 pS (n=5) (FIG. 2aii) to 165+1-6.1 p5 (n=3) (FIG. 2bii). Native sodium activated potassium channels also have subconductance states [Wang, 1991 #38], and are more strongly inwardly rectifying when K⁺ o>K⁺ᵢ[Luk, 1990 #11] or when [Na⁺]ᵢ, is high [Wang, 1991 #38; Egan, 1992 #5]. Subconductance states were also observed in rSlo2 (FIG. 2ai and FIG. 2bi, arrows). The inward rectification of rSlo2 was also much weaker in 160 mM K⁺ₒ and K⁺ᵢ(FIG. 2bii and 2biii) compared to 80 mM K⁺ₒ and K⁺ᵢ(FIG. 2aii and 2aiii), due to the absence of Na⁺ᵢ in the experiments with 160 mM K⁺₀ and K⁺ᵢ.

Whole Cell Currents

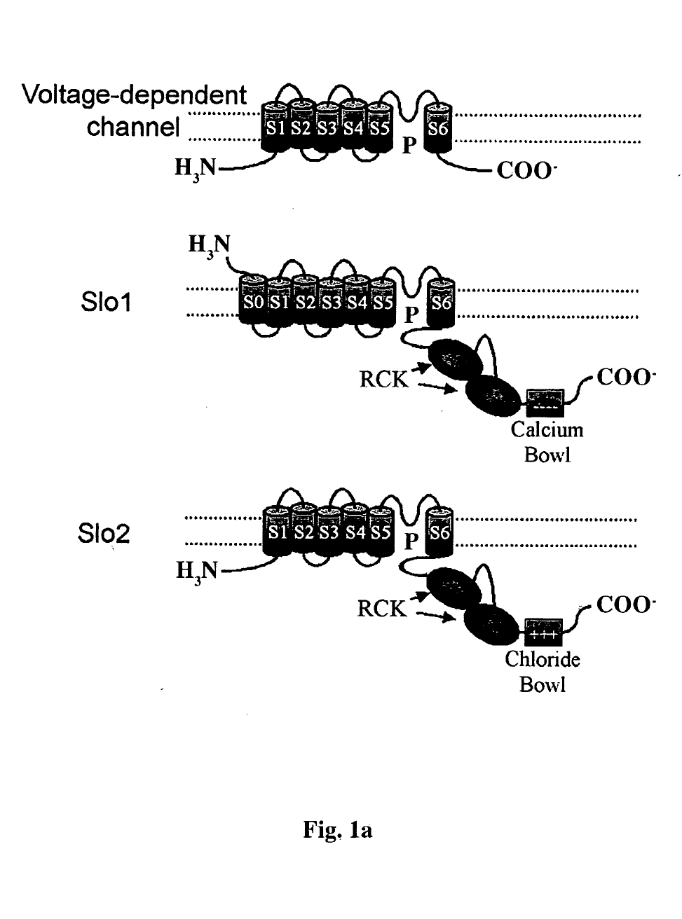

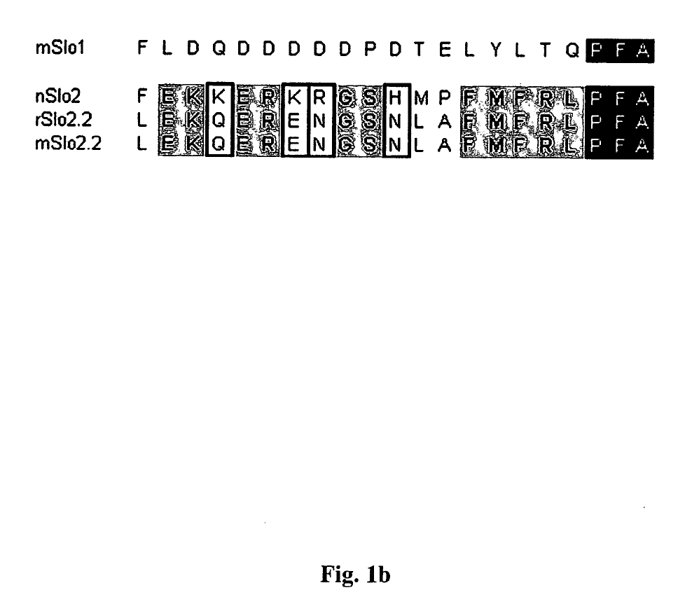

Whole-cell recordings from neurons indicate that I (sodium activated potassium channels) may be an outwardly rectifying current under physiological conditions (low K⁺ₒ high K⁺ᵢ) [Schwindt, 1989 #4; Dale, 1993 #8]. We also observed outwardly rectifying currents in rSlo2 injected oocytes from cell-attached macropatches using low [K⁺]ᵢ external (pipette) solution (FIG. 3ai and 3aiii). Two-electrode voltage clamp (TEVC) of rSlo2 injected oocytes also produced similar outwardly rectifying currents (FIG. 3b). There are obvious differences in activation kinetics between the currents recorded in FIG. 3ai and 3bi and currents recorded from inside-out patches in symmetrical K⁺(FIG. 1ai). The delayed onset of activation in the cell-attached and TEVC modes could be due to several factors such as modification of channel kinetics by an intracellular factor.

Co-Activation By Chloride

An unusual property of Slo-2, the C. elegans orthologue of rSlo2, is the dependence on chloride ions (“Cl⁻ᵢ”) for activation. This dependence was confirmed by testing rSlo2 for Cl⁻ᵢ dependence, and our discovery that rSlo2, like Slo-2, is indeed activated by Cl⁻ᵢ(FIG. 4a). This activation by Cl⁻ᵢ was seen only when Na⁺ᵢ is also present (FIG. 4bi). This is similar to C. elegans Slo-2, where Cl⁻ᵢ activation is seen only in the presence of Ca²⁺ᵢ.

In C. elegans Slo-2 the dual requirement for Ca²⁺, and Cl⁻ᵢ is absolute; no significant macroscopic currents are seen in the absence of either ion [Yuan, 2000 #10]. In contrast, in rSlo2, Cl⁻ᵢ enhances potassium ion channel activation, often occurring in the presence of Na⁺ᵢ alone. This dual activation shows cooperativity (synergy). That is, the activity of the channel in the presence of both ions is greater than the sum of the activity of the channel in the presence of in either ion alone. (FIG. 4bi and 4bii). This cooperative effect between Na⁺ᵢ and Cl⁻ᵢ is physiologically relevant. Serving as a mechanism to amplify the channel's response to ischemia. During ischemia or hypoxia, [Na⁺]ᵢ increases due to a failure of the Na⁺/K⁺-ATPase to pump Na⁺ᵢ out of the cell [Kameyama, 1984 #1]; and Cl⁻ᵢ also increases during ischemia [Lai, 1998 #23]; demonstrating that an increase in [Cl⁻]ᵢ dramatically boosts the sensitivity of rSlo2 to Na⁺ᵢ, allowing Slo2 to be activated by lower concentrations of Na⁺ᵢ. Further, an increase in [Na⁺]ᵢ is usually accompanied by an increase in the concentration of the counterion Cl⁻ᵢ. The Cl⁻/HCO₃⁻ exchanger mechanism for increased Cl⁻ᵢ is the accepted mechanism for increased [Cl⁻]ᵢ in cardiomyocytes, not passive influx of Cl⁻ᵢ as in epithelial cells. A similar mechanism occurs in neurons [Haddad, 1993 #25] where Slo2 plays a similar protective role.

Additionally, C. elegans Slo-2 is activated by Ca²⁺ᵢ˜ and Cl⁻ᵢ, instead of Na⁺ᵢ˜ and Cl⁻ᵢ. This difference in ionic sensitivity between C. elegans Slo-2 and rSlo2 is due to the relatively greater reliance on Ca²⁺ to carry inward current in C. elegans.

Human hSlo2.2 Gene

The isolated human gene, hSlo2.2, with a primary amino acid sequence, SEQ ID NO:1, was discovered. The inventors determined the tissue distribution of hSlo2.2 using RT-PCR. This hSlo2.2 gene was detected most abundantly in brain, heart, kidney, and testis, (FIG. 5), indicating that hSlo2.2 is specialized primarily for functions within these tissue types.

C. Elegans Slo-2 is a Major Current Regulator in Muscle

In order to determine what role Slo-2 plays in vivo, the mutants generated two Slo-2 deletion mutants in C. elegans (Wei et al., 2002). Using a recently developed C. elegans cell culture technique (Christensen, et al, 2002). We recorded from wild-type and Slo-2 (nf100) mutant cells in culture. We discovered that currents recorded from muscle cells consisted of two major components, one transient, and one delayed. Currents recorded from the Slo-2 mutant had a dramatically reduced delayed component. To quantify the amplitude of the delayed component in wild-type and Slo-2 mutant cells, we measured the current near the end of a 900 ms voltage step in both wild-type and Slo-2 mutants. The average size of the delayed outward current at +60 mV in wild-type cells was 37.6+/−5 pA/pF (n=22) compared to 10.0+/−1 pA/pF (n=15) in Slo-2 mutant cells. This difference is shown in representative current traces in FIG. 7a. In addition to quantitative differences between outward currents in wild-type and Slo-2 mutant cells, there were qualitative differences as well. In 100% of the Slo-2 mutant cells, the transient current was the major outward component (FIG. 7aii). In wild-type cells, single channel openings of a large conductance channel were observed which resulted in a choppy, high noise level of current traces. However, in the mutant cells, openings of large conductance single channels were never observed and the noise level of current recordings was lower (n=1 5). These results suggest that Slo-2 enclodes high conductance channels and that these channels constitute the major component of the delayed outward current in these cells.

Because the cultured muscle cells were embryonic in origin, we confirmed our results in adult body wall muscle cells using the filleted worm preparation technique (Richmond and Jorgensen, 1999; Wang et al., 2001). As in cultured cells, a comparison of adult wild-type and mutant cells showed that the average size of the delayed outward current in wild-type cells was much larger than in mutant cells (130.1+/−21 pA/pF (n=5) compared to 48.8+/−10 pA/pF (n=6) in mutant cells) (FIG. 7b). As with cells in culture, the delayed, non-inactivating current was the major component in most wild-type cells (FIG. 7bi), but in 100% of mutant Slo-2 cells, the transient current was the major component (FIG. 7bii).

Slo-2 Mutants Are Hypersensitive To Hypoxic Death

Mammalian Slo2 is found in both cardiomyocytes (Kameyama et al., 1984; Luk and Carmeliet, 1990) and neurons (Bader et al., 1985; Dryer et al., 1989; Schwindt et al., 1989; Egan et al., 1992b; Dale, 1993; Safronov and Vogel, 1996; Bischoff et al., 1998) where it may serve a protective role against ischemia by reducing membrane excitability during hypoxic stress (Kameyama et al., 1984; Dryer, 1994). Since [Cl⁻]ᵢ as well as [Na⁺]ᵢ and [Ca²⁺]ᵢ rise during hypoxia (Kameyama et al., 1984; Lai and Nishi, 1998), activation of rSlo2 by Cl⁻ᵢ as well as Na⁺ᵢ may boost its ability to sense and react to hypoxic conditions. Slo-2 in C. elegans could offer analogous protection against hypoxia by sensing Ca²⁺ᵢ and Cl⁻ᵢ. Using an assay for hypoxic death in C. elegans (Scott et al., 2002), we compared the response of wild-type animals to Slo-2 mutants and a mutant of the Slo-1 gene used as a control; Slo-1 encodes a Ca²⁺ but not a chloride activated potassium channel (Wang et al., 2001). After a 16 hour incubation in a hypoxic chamber, 58+/−6% of wild-type animals died compared to 86+/−2% of Slo-2 mutants [both deletion strains combined: 87+/−2% of Slo-2 (nf101), 84+/−3% of Slo-2 (nf101)]. t-test showed that Slo-2 mutants were significantly more sensitive to hypoxia than wild type (P<0.001), whereas a Slo-1 control was not significantly different (P=0.818).

Role of Sodium Activated Potassium Channels

A long-standing controversy concerning the role of sodium activated potassium channels is the ability of the channels to function under physiological concentrations of Na⁺ᵢ. Reports of the Na⁺ sensitivity of sodium activated potassium channels vary widely, ranging from an EC₅₀ of 7.3 mM to 80 mM (Dryer, 1994). During ischemia or hypoxia, [Na⁺]ᵢ increases due to a failure of the Na⁺/K⁺-ATPase pump to remove Na⁺ from the cell (Kameyama et al., 1984); [Cl⁻]ᵢ also increases during isehemia. Nevertheless, even though co-activation by Cl⁻makes it seem more likely that the rSlo2 channel will be, at least, partially active under conditions of isehemia, this question has not been settled and requires further investigation. The intracellular concentration of chloride is reported to rise to approximately 55 mM in some instances of simulated cardiac isehemia (Lai and Nishi, 1998). Intracellular sodium ion has been reported to range from approximately 19 mM (Nakamura et al, 1999; Kline et al, 1992) to 72 mM (Pogorelov et al, 2002). We undertook tests within these values in FIG. 5ii. From the plot in 20 mM concentrations of sodium ions we estimate that the current will reach approximately 0.18 of I max at 55 mM Cl⁻. Sensitivity to chloride ion has not yet been demonstrated for native sodium activated potassium channels. In one instance, sodium activated potassium channels were analyzed in the presence of high levels of chloride, and those channels did not show a particularly high sensitivity to sodium ion (Dryer, Fujii & Martin, 1989). Because more than one gene may encode sodium activated potassium channels, and because other factors such as alternative RNA splicing and accessory subunits may increase the functional heterogeneity of sodium activated potassium channels, many questions regarding the physiological roles and sodium sensitivity of this intriguing class of channels remain.

Only a small number of Slo-2 channels may ever be active, except under the most extreme conditions. The C. elegans data shows that Slo-2 represents such a large potential outward current in the muscle cells that its complete activation would almost certainly abolish any active inward current response. Conceivably, such a large number of Slo-2 high conductance channels could furnish a cell with a kind of safety net to provide early, fail safe interception and amelioration of the detrimental effects of hypoxia and/or ischemia, but only a tiny fraction of these channels need be active to respond to the initial effects of hypoxia. A similar mechanism may occur in neurons (Haddad and Jiang, 1993), where sodium activated potassium channels play a protective role in intercepting the early effects of ischemia. Sodium activated potassium channels are abundant in many mammalian cells, and act similarly in that usually, only a tiny fraction of this large potential conductance is ever used. Channels held in reserve, which are not usually active, represent the last line of defense against the more pathological conditions that accompany ischemia, namely a rise in the bulk concentrations of intracellular ions that can lead to a fatal osmotic imbalance.

Mechanism of NA⁺/Cl⁻ Activation

The activation of rSlo2 by Na⁺ and its C. elegans orthologue, Slo-2, by Ca²⁺ poses question as to what mechanism of Ca²⁺/Cl⁻ or Na⁺/Cl⁻ sensing. Site directed mutagenesis previously showed that a region in the tail of the C. elegans Slo-2 channel, the chloride bowl is important for sensing [Ca²⁺]ᵢ and [Cl⁻]ᵢ (Yuan et al., 2000), and the calcium bowl, an analogous region in the BK Slo1 channel is important for sensing [Ca²⁺]ᵢ(Schreiber and Salkoff, 1997; Schreiber et al., 1999). The calcium bowl consists of a string of negatively charged amino acid residues that are perfectly conserved in all Slo1 channels. These negative charges may help to coordinate Ca²⁺ in Slo1 channels. In contrast, the chloride bowl consists of a string of positively charged residues in many positions which correspond to negative charges in the chloride bowl (FIG. 1b). Whether these residues help coordinate Cl⁻and perhaps even cooperatively bind Ca²⁺, remains to be determined. Nevertheless, site-directed changes in the chloride bowl produced one mutant that significantly reduced sensitivity of gating to both Ca²⁺ and Cl⁻, and another mutant that eliminated sensitivity to Ca²⁺ and Cl⁻ altogether (Yuan et al., 2000). Hence, differences in the amino acid sequence between the mammalian and C. elegans sodium activated potassium channels in this region may conceivably relate to differences between C. elegans Slo-2 and rSlo2 with respect to Ca²⁺/Cl⁻ versus Na⁺/Cl⁻ activation (FIG. 1b).

In Slo1 channels, a second region possibly corresponding to a RCK domain (Jiang et al., 2001; Jiang et al., 2002) has recently been discovered to also be important for sensing [Ca²⁺]ᵢ(Jiang et al., 2001; Jiang et al., 2002; Xia et al., 2002). Conceivably, one or more RCK domains are also present in hSlo2.2 at corresponding sites, and such regions are important for determining the gating properties in hSlo2.2 channels.

Evolution of Na⁺/Cl⁻ Activated Channel

The difference in ion-dependence of gating between the Slo-2 orthologues in C. elegans and hSlo2.2 in humans may reflect a more important role that Ca²⁺ plays in C. elegans since a voltage-gated sodium channel is apparently absent (Bargmann, 1998). It has been suggested that Ca²⁺ channels may have preceded Na⁺ channels in evolution (Hille, 1992). Perhaps a Ca²⁺/Cl⁻ activated Slo channel was present in a common ancestor to C. elegans and vertebrates. Following the evolution of Na⁺ channels, this ancestral Slo channel may have evolved to sense Na⁺/Cl⁻ instead of Ca²⁺/Cl⁻.

Protection Against Hypoxia—A Conserved Role

Using a hypoxic death assay, Slo-2 mutants showed hypersensitivity to hypoxic death, suggesting that Slo-2 in C. elegans protects against the detrimental effects of hypoxia. During hypoxia, an increase in intracellular concentrations of sodium ions is followed by an increase in intracellular concentrations of calcium and cloride ions. A likely scenario is that in humans, the rise in sodium and chloride ions activates hSlo2.2 encoded sodium activated potassium channels, while in C. elegans, the rise in calcium and chloride ions causes activation of the rSlo-2 encoded calcium activated potassium channels. The resulting activation of hSlo2.2 may protect the cell by hyperpolarizing the resting potential, limiting electrical activity, and improving Na⁺ transport.

Clinical Relevance

The identification of the molecular identity of sodium activated potassium channels will allow for the development of specific agonists and antagonists. New therapies might be developed for treatment of any condition involving hypoxia including angina, stroke, cardiac isehemia, cardiac arrhythmias, brain trauma, fetal hypoxia, and hypoxia during organ transplantation. In this latter role, preconditioning organs for transplant using K_{ATP} channel openers now appears to be highly effective (Zhang et al., 2001). Indeed, hypoxic preconditioning may prove to be the key to success in many operations involving brain and heart. Because sodium-activated potassium channels are widespread, openers for this channel may be equally or more effective than openers for K_{ATP} in clinical medicine.

The core domain of hSlo2.2 (amino acids 200-600) is useful to identify members of the human Slo gene family, and also homologues of hSlo2.2. For example, potassium channel proteins that share at least about 92% or greater amino acid identity in the core domain are hSlo2.2 proteins.

Our isolation of the hSlo2.2 protein encoded by the hSlo2.2 gene, for the first time provides a useful means for assaying for compounds that modulate, i.e. increase or decrease the ion channel activity of sodium activated potassium channels, which are involved in cardiomyocyte and neuron physiology.

Additionally hSlo2.2 nucleic acids and these exercised proteins served proteins have utility widely in that they are useful for testing inhibitors or activators of hSlo2.2 using in vitro assays, e.g, expressing hSlo2.2 in cells or cell membranes and then measuring flux of ions through the channel. In an aspect, Inhibitors or activators identified using hSlo2.2 can be used therapeutically to treat conditions related to cardiomyocyte and neuron physiology, including ischemic conditions and digitalis toxicity.

We have discovered that hSlo2.2 expression provides a convenient diagnostic marker in cells for ischemia resistance.

Cardiomyocytes and neurons lacking hSlo2.2 expression may be indicative of cells that lack the capability to withstand more than the most basic conditions incident to ischemia or digitalis toxicity, which are essential to cellular survival. Antibodies or other probes for hSlo2.2 can be also used in vitro as diagnostic tools to examine hSlo2.2 expression.

hSlo2.2 can also be used to study cardiomyocyte and neuron physiology in vitro, e.g., the ability of these cells to withstand prolonged conditions of ischemia and digitalis toxicity that are detrimental to the survival of cardiomyocytes and neurons.

Portions of the hSlo2.2 nucleotide sequence may be used to identify homologues of the ion channel, as well as variants or mutations of the channel that may be associated with disease. This identification can be made in vitro or by using the sequence information in a computer system for comparison with other nucleotide sequences. Similarly, these portions of hSlo2.2 nucleotide sequence may be used to determine the presence of a hSlo2.2 channel mRNA or channel protein in a particular tissue of interest. Information derived from the hSlo2.2 nucleotide sequence may also be used to identify the chromosomal localization of the hSlo2.2 gene or genes using chromosomal panels, radiation hybrid screening, fluorescent in situ hybridization methods (FISH), or by comparison of the sequence with computer nucleic acid databases. The hSlo2.2 channel or fragments thereof may also be used to treat diseases using gene therapy. In an aspect, hSlo2.2 nucleotide sequence information may also be used to construct models of the sodium activated potassium channel protein in a computer system, these models subsequently used to predict compounds that can modulate channel function.

After reading the specification, once in the possession of one of these novel genes, or an equivalent gene, in an aspect one inserts the gene into an appropriate expression vector in order to obtain functional expression of the encoded channel polypeptides in a heterologous system. Such procedures are used to characterize the functional properties of the channels and to evaluate them as targets for pharmaceuticals. In addition, the channel polypeptides can be overproduced in a suitable host cell and then purified from the other cell proteins using methods available to those skilled in the art.

It is noted that our discovery encompasses all forms of the genes disclosed herein, including, the genes in cDNA form and in genomic (unspliced) configuration.

After reading this specification, one of skill in the art will appreciate that the characteristic sequences need not be identical at the DNA level or the protein levels to those presented herein. Through the ordinary course of evolutionary mutation and somatic change in individuals, certain nucleotide and/or amino acid substitutions and small additions or deletions not affecting the activity of the proteins encoded thereby are to be expected. Degeneracy of the genetic code permits “wobble” at the third bases of codons. Thus, some nucleotide changes will have no bearing at all on the amino acids encoded by the genes. The inventors intend that the claims encompass all such modifications and variations of the sequences presented, without regard to the species of origin of the gene or protein or indeed whether isolated from a living organism or deliberately engineered in vitro using recombinant DNA methods available to the genetic engineer.

Unless otherwise indicated, nucleic acids are written left to right in 5′ to 3′ orientation; amino acid sequences are written left to right in amino to carboxy orientation, respectively. Numeric ranges are inclusive of the numbers defining the range.

The terms defined below are more fully defined by reference to the specification as a whole.

The terms “isolated” “purified” or “biologically pure” refer to a material or composition that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated hSlo2.2 nucleic acid is separated from open reading frames which flank the hSlo2.2 gene and encode proteins other than hSlo2.2. As used herein, the term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The terms “nucleic acid” “probe”, or “primer” includes reference to a deoxyribonucleotide or ribonucleotide polymer in either single- or double-stranded form, and unless otherwise limited, encompasses known analogues of natural nucleotides that hybridize to nucleic acids in manner similar to naturally occurring nucleotides. Unless otherwise indicated, a particular nucleic acid sequence includes the perfect complementary sequence thereof. Eukaryotic nucleic acids are nucleic acids from eukaryotic cells, preferably cells of multicellular eukaryotes.

As used herein, the terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein, a “polynucleotide” refers to a chain of nucleotides. Preferably, the chain has from about five to 10,000 nucleotides, more preferably from about 50 to 3,500 nucleotides. The term “probe” refers to a polynucleotide sequence capable of hybridizing with a target sequence to form a polynucleotide probe/target complex under hybridization conditions. A “target polynucleotide” refers to a chain of nucleotides to which a polynucleotide probe can hybridize by base pairing. In some instances, the sequences will be completely complementary (no mismatches) when aligned; in others, there may be up to a 10% mismatch.

As used herein, the term “subsequence” in the context of a referenced nucleic acid sequence includes reference to a contiguous sequence from the nucleic acid having fewer nucleotides in length than the referenced nucleic acid. In the context of a referenced protein, polypeptide, or peptide sequence (collectively, “protein”), “subsequence” refers to a contiguous sequence from the referenced protein having fewer amino acids than the referenced protein.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, “Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill in the art will recognize that each codon in a nucleic acid (except AUG, which is ordinarily the only codon for methionine) can be modified to yield a functionally identical molecule. Accordingly, each silent variation of a nucleic acid that encodes a polypeptide is implicit in each described sequence.

As to amino acid sequences, one of skill in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

1) Alanine (A), Serine (5), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

(see, e.g., Creighton, Proteins (1984)).

A “label” is a composition detectable by spectroscopic, photochemical, biochemical, immunochemical, chemical or electrochemical means. For example, useful labels include ³²P., fluorescent dyes, electron-dense reagents, enzymes (e.g., as commonly used in an ELISA), biotin, dioxigenln, or haptens and proteins for which antisera or monoclonal antibodies are available (e.g., the peptide of at least one of SEQ ID NO:1 and SEQ ID NO:2 can be made detectable, e.g., by incorporating a radio-label into the peptide. and used to detect antibodies specifically reactive with the peptide).

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C; or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

A “labeled nucleic acid probe or oligonucleotide” is one that is bound, either covalently, through a linker, or through ionic, van der Waals or hydrogen bonds to a label such that the presence of the probe may be detected by detecting the presence of the label bound to the probe.

“Amplification” primers are oligonucleotides comprising either natural or analogue nucleotides that can serve as the basis for the amplification of a select nucleic acid sequence. They include, e.g., polymerase chain reaction primers and ligase chain reaction oligonucleotides. Amplification primers are used to “amplify” a target nucleic acid sequence.

The term “recombinant” when used with reference to a cell, or protein, nucleic acid, or vector, includes reference to a cell, protein, or nucleic acid, or vector, that has been modified by the introduction of a heterologous nucleic acid or the alteration of a native nucleic acid to a form not native to that cell, or that the cell is derived from a cell so modified. Thus, for example, recombinant cells express genes and proteins that are not found within the native (non-recombinant) form of the cell or express native genes that are otherwise abnormally expressed, under expressed or not expressed at all.

The phrase “encodes a protein which could be encoded by a nucleic acid that selectively hybridizes under moderate stringency hybridization conditions to a sequence” in the context of nucleic acids refers to those nucleic acids encoding naturally occurring proteins or derivatives of natural proteins, but which are deliberately modified or engineered to no longer hybridize to the protein of natural origin under the stated conditions.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., USA).

The phrase “substantially identical,” in the context of two nucleic acids or polypeptides, refers to sequences or subsequences that have at least 60%, preferably 80%, most preferably 90-95% nucleotide or amino acid residue identity when aligned for maximum correspondence over a comparison window as measured using one of the following sequence comparison algorithms or by manual alignment and visual inspection. This definition also refers to the complement of a test sequence, which has substantial sequence or subsequence complementarity when the test sequence has substantial identity to a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection.

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul et al., J. Mol. Biol. Biol. 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff & Henikoff, Proc. Nat'l. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Na'l Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization.

Nucleic acids that do not hybridize to each other under stringent conditions are substantially identical if the polypeptides that they encode are substantially identical. This occurs, for example, when a copy of a nucleic acid is created using the maximum codon degeneracy permitted by the genetic code.

Exemplary “moderately stringent hybridization conditions” include a hybridization in a buffer of 40% formamide, 1 M NaCI, 1% SDS at 37° C., and a wash in 1X SSC at 45° C. A positive hybridization is at least twice background. Those of ordinary skill will readily recognize that alternative hybridization and wash conditions can be utilized to provide conditions of similar stringency. Nucleic acids which do not hybridize to each other under moderately stringent or stringent hybridization conditions are still substantially identical if the polypeptides which they encode are substantially identical. This may occur, e.g., when a copy of a nucleic acid is created using the maximum codon degeneracy permitted by the genetic code.

An indication that two polypeptide sequences are substantially identical is that one peptide is immunologically reactive with antibodies raised against the second peptide. Thus, a polypeptide is substantially identical to a second polypeptide, for example, where the two peptides differ only by a conservative substitution. An indication that two nucleic acid sequences are substantially identical is that the polypeptide which the first nucleic acid encodes is immunologically cross reactive with the polypeptide encoded by the second nucleic acid.

The phrase “a sequence encoding a gene product” refers to a nucleic acid that contains sequence information, e.g., for a structural RNA such as rRNA, a tRNA, the primary amino acid sequence of a specific protein or peptide, a binding site for a transacting regulatory agent, an antisense RNA or a ribozyme. This phrase specifically encompasses degenerate codons (i.e., different codons which encode a single amino acid) of the native sequence or sequences which may be introduced to conform with codon preference in a specific host cell.

The term “antibody” also includes antigen binding forms of antibodies (e.g., Fab, F(ab)₂). The term “antibody” refers to a polypeptide substantially encoded by an immunoglobulin gene or immunoglobulin genes, or fragments thereof which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist e.g., as intact immunoglobulins or as a number of well characterized fragments produced by digestion with various peptidases. Thus, for example, pepsin digests an antibody below the disulfide linkages in the hinge region to produce F(ab)′₂ a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially a Fab with part of the hinge region (see, e.g., Fundamental Immunology (Paul ed., 3d ed. 1993). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments such as single chain Fv, chimeric antibodies (i.e., comprising constant and variable regions from different species), humanized antibodies (i.e., comprising a complementarity determining region (CDR) from a non-human source) and heteroconjugate antibodies (e.g., bispecific antibodies).

An “anti-hSlo2.2” antibody includes an antibody or antibody fragment that specifically binds a polypeptide encoded by an hSlo2.2 gene, cDNA, or a subsequence thereof. The antibody can be either a monoclonal or polyclonal antibody.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The term “immunoassay” is an assay that uses an antibody to specifically bind an analyte. The immunoassay is characterized by the use of specific binding properties of a particular antibody to isolate, target, and/or quantify the analyte.

The phrase “specifically (or selectively) binds to an antibody” or specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. For example, antibodies raised to hSlo2.2 with the amino acid sequence encoded in at least one of SEQ ID NO:1 and SEQ ID NO:2, respectively, can be selected to obtain polyclonal antibodies specifically immunoreactive with that protein and not with other proteins, except for polymorphic variants. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

The phrase “functional effects” in the context of assays for testing compounds affecting the channel includes the determination of any parameter that is indirectly or directly under the influence of the channel. It includes changes in ion flux, membrane potential, voltage gating, pH, Cl⁻and Na⁺ sensitivity and also includes other physiologic effects such increases or decreases of transcription or hormone release.

By “determining the functional effect” is meant examining the effect of a compound that increases or decreases ion flux on a cell or cell membrane in terms of cell and cell membrane function. The ion flux can be any ion that passes through the channel and analogues thereof, e.g., potassium, rubidium, sodium. Preferably, the term refers to the functional effect of the compound on hSlo2.2 channel activity, e.g., changes in ion flux including radioisotopes, current amplitude, voltage gating, pH, Cl⁻and Na⁺ sensitivity, and the like. Such functional effects can be measured by any means known to those skilled in the art, e.g., patch clamping, whole cell currents, pH, Na+ and voltage sensitive dyes, radioisotope efflux, inducible markers, and the like.

“Inhibitors” and “activators” of the ion channel experienced by hSlo2.2 refer to inhibitory or activating molecules identified using in vitro assays for hSlo2.2 function. As used herein the term “Inhibitors” include compounds that decrease, block, prevent, delay activation, inactivate, desensitize, or down regulate the channel. As used herein, the term “activators” includes compounds that increase, open, activate, facilitate, enhance activation, sensitize or up regulate ion channel activity.

Useful nonlimiting assays for inhibitors and activators include expressing hSlo2.2 in a cell or membrane and determining changes in polarization (i.e., electrical potential). Useful non-limiting methods of measuring changes of cell membrane polarization include voltage-clamp techniques, determination of whole cell currents, radiolabeled rubidium flux assays, and fluorescence assays using voltage-sensitive dyes (as described hereinafter in more detail).

Samples or assays that are treated with a potential hSlo2.2 activator or inhibitor are compared to control samples without the inhibitor, to examine the extent of inhibition. Control samples (untreated with inhibitors) are assigned a relative hSlo2.2 activity value of 100%. Inhibition of hSlo2.2 is achieved when the hSlo2.2 activity value relative to the control is about 90%, preferably 50%, more preferably 25%. Activation of hSlo2.2 is achieved when the hSlo2.2 activity value relative to the control is 110%, more preferably 150%, more preferable 200% higher.

By “host cell” is meant a cell that contains an expression vector and competently supports the replication or expression of the expression vector. Host cells may be prokaryotic cells such as E. coli, or eukaryotic cells such as yeast, insect, amphibian, e.g., Xenopus, or mammalian cells such as CHO, HeLa and the like. The hSlo2.2 channel can also be expressed in a cell membrane derived from such a cell.

“Biological sample” as used herein includes a sample of biological tissue or fluid that contains a hSlo2.2 channel protein or nucleic acid encoding the corresponding hSlo2.2 channel protein. Such samples include, but are not limited to, cardiomyocytes, oocytes and neuron, brain, kidney, testis and heart tissue. Biological samples may also include sections of tissues such as frozen sections taken for histological purposes. A biological sample is typically obtained from a eukaryotic organism, preferably a multicellular eukaryotes such as insect, protozoa, birds, fish, reptiles, and preferably a mammal such as rat, mice, cow, dog, guinea pig, or rabbit, and most preferably a primate such as macaques, chimpanzees, or humans.

By “conductance” is meant electrical conductance. Electrical conductance is conveniently measured in Siemens (1/ohm =mho). Unitary conductance is determined by measuring single channel currents using a patch clamp protocol under conditions set forth in (i.e., in a Xenopus oocyte) using a symmetrical potassium ion concentration of 160 mM (see generally, Hille, Ionic Channels of Excitable Membranes (2d ed.) In the context of the present invention, “conductance” refers to the unitary electrical conductance of a single homomeric protein of the referenced hSlo2.2 channel protein.

“Functional tetrameric form” refers to expression of a hSlo2.2 protein or monomer in which a plurality of the hSlo2.2 proteins are assembled to form, by themselves or in conjunction with other endogenous Xenopus oocyte molecules, an hSlo2.2 potassium channel. Expression within a Xenopus oocyte is disclosed in the Examples provided herein. Typically the hSlo2.2 channel is a homotetramer formed of four hSlo2.2 monomer proteins.

By “contiguous amino acids from” in the context of a specified number of amino acid residues from a specified sequence, is meant a sequence of amino acids of the specified number from within the specified reference sequence which has the identical order of amino acids each of which is directly adjacent to the same amino acids as in the reference sequence.

As used herein, the term “Na⁺ sensitive potassium channel” or “hSlo2.2 channel” includes a membrane channel which is voltage-gated, Na⁺ sensitive (e.g., with increased activity as measured by increased current amplitude above a Na⁺ concentration of about 1 mm), has a unitary conductance of from about 80 pS to about 120 pS when measured under a symmetrical potassium concentration of 160 mM in a Xenopus oocyte using conditions provided herein. A hSlo2.2 channel comprises multiple hSlo2.2 channel proteins as subunits, typically four hSlo2.2 channel proteins (e.g., full length or substantially full length hSlo2.2 channel proteins).

As used herein, the term “Na⁺ sensitive potassium channel protein” or “hSlo2.2 channel protein” means a polypeptide of a molecular weight of between about 120-156 kDa. These proteins serve as monomers of the hSlo2.2 channel. Thus, a hSlo2.2 channel protein can have the functional characteristics to form a heteromeric or homomeric protein with the functional characteristics of a hSlo2.2 channel, or be a peptide fragment thereof. This term includes both recombinant and naturally occurring forms of hSlo2.2. Both recombinant and naturally occurring hSlo2.2 can be used in the methods of the invention described herein, e.g., in assays to identify inhibitors or activators of hSlo2.2. The term hSlo2.2 therefore refers to polymorphic variants, alleles, mutants, and interspecies variants of hSlo2.2 that: (1) have greater than 60% amino acid sequence identity to amino acids 200-600 of a hSlo2.2 core domain; or (2) bind to antibodies raised against an immunogen comprising an amino acid sequence selected from the group consisting of SEQ ID NO:1, SEQ ID NO:2, and conservatively modified variants thereof; or (3) specifically hybridize under stringent hybridization conditions to a sequence selected from the group consisting of SEQ ID NO:1 and SEQ ID NO:2, and conservatively modified variants thereof; or (4) are amplified by primers that specifically hybridize under stringent hybridization conditions to the same sequence as a primer set consisting of at least one of SEQ ID NO:1 and SEQ ID NO:2.

As used herein, the term “Na⁺ sensitive” refers to a characteristic of hSlo2.2 channels, where the channels have increased current amplitude in response to changes in intracellular Na⁺ concentrations Na⁺ (Na⁺ᵢ). Typically, Na⁺ sensitive channels show increased current amplitude above approximately Na⁺ᵢ of 1 mM. Na⁺ sensitivity can be measured using a number of assays. For example, single channel recordings are made from inside out patches that have been perfused with saline of different concentrations of Na⁺ and the open probability of the channel vs. the concentration of Na⁺ is plotted to determine Na⁺ concentration sensitivity. In another example, macroscopic current is examined with an inside out patch perfused with saline of varying concentrations of Na⁺, and the amplitude of the current is measured.

Na⁺ Sensitive Potassium Ion Channels

The present discovery provides isolated Na⁺ sensitive potassium channel proteins. The isolated Na⁺ sensitive potassium channel (hSlo2.2) proteins of the present invention comprise at least N amino acids from any one of the sequences selected from the group consisting of: at least one of SEQ ID NO:1, and SEQ ID NO:2, and conservatively modified variants thereof, where N is an integer and is any one of the integers selected from the group consisting of from about 10 to about 600 and the sequence is unique to the protein of origin. Useful nonlimiting examples of integers include integers 1,2,3,4,5,6,7,8,9,10 and the like.

Typically, the Na⁺ sensitive potassium channel proteins are at least about 100, 200, 300, 400, or 500 amino acids in length, and most preferably the full length of hSlo2.2 or conservatively modified variants thereof. Smaller hSlo2.2 peptide fragments of 15, 25, 35, or 50 amino acids are used for epitope mapping and antibody production. Thus, the present invention provides full-length and subsequences of hSlo2.2 and conservatively modified variants of hSlo2.2. A “full-length” amino acid sequence of hSlo2.2 means the sequence of at least one of SEQ ID NO:1 and SEQ NO:2. Full-length sequences of hSlo2.2 include those that encode a protein comprising SEQ ID NOS:1 and 2, which represent conservatively modified variants of hSlo2.2. The Na⁺ sensitive potassium channel proteins and peptides of the present invention can be used as immunogens for the preparation of immunodiagnostic probes for assessing increased or decreased expression of Na⁺ sensitive potassium channels in drug screening assays.

Na⁺ sensitive potassium channel proteins of the present invention include proteins that have substantial identity (i.e., similarity) to a Na⁺ sensitive potassium channel protein of at least N amino acids from any one of the sequences selected from the group consisting of at least one of SEQ ID NO:1 and SEQ ID NO:2 and SEQ ID NO: 2 and conservatively modified variants thereof, where N is any one of the integers selected from the group consisting of 10 to 600. Generally, the Na⁺ sensitive potassium channel proteins are at least 100, preferably at least 200, more preferably at least 400, and most preferably at least 600 amino acid residues in length. Typically, the substantially similar or conservatively modified variant of the Na⁺ sensitive potassium channel protein (hSlo2.2) is a eukaryotic protein, preferably from a multicellular eukaryotes such as insects, protozoans, birds, fishes, amphibians, reptiles, and mammals.

The hSlo2.2 channel proteins which are substantially identical to, or a conservatively modified variant of, a hSlo2.2 channel protein comprising a sequence selected from at least one of SEQ ID NO:1 and SEQ ID NO:2, will specifically react, under immunologically reactive conditions, with an immunoglobulin reactive to a hSlo2.2 channel protein selected from the group consisting of at least one of SEQ ID NO:1 and SEQ ID NO:2.

Alternatively, the hSlo2.2 channel proteins which are substantially identical to, or are a conservatively modified variant of, a hSlo2.2 channel protein having a sequence selected from at least one of SEQ ID NO:1 and SEQ ID NO:2, will comprise an amino acid sequence which has any one of the values from about 60% to 100% similarity to a comparison window within the core domain (or “core region”) of an hSlo2.2 channel protein selected from the group consisting of at least one of SEQ ID NO:1 or SEQ ID NO:2, and SEQ NO:3. The core domain corresponds to the 50-56 transmembrane regions of hSlo2.2, e.g., amino acids 200-600 of hSlo2.2. A subsequence of the core domain has a length of any one of the numbers from 10 to the length of a core domain sequence of SEQ ID NOS:1 or 2. Preferably, hSlo2.2 channel proteins comprise an amino acid sequence having at least 90% similarity over a comparison window of 20 contiguous amino acids from within the core domain sequence.

The present discovery provides functional isolated hSlo2.2 channel proteins and subsequences thereof. Functional hSlo2.2 channels of the present invention have a unitary conductance of between about 60 and about 180 pS (as measured in Xenopus oocytes with symmetrical potassium concentrations), and molecular weights between about 120 and about 156 kDa for each of the hSlo2.2 channel protein monomers that make up the hSlo2.2 channel. Unitary conductance may be conveniently determined using single channel or macroscopic channel inside-out or outside-out patch clamp configurations or whole cell recordings (see Wei et al., 1994, supra). These configurations are particularly indicated for the study of the biophysics of ionic channels (kinetics, conductivity, selectivity, mechanism of permeation and block). Patch clamp and whole cell recording methods are well known in the art (see, e.g., Franciolii, Experientia, 42:589-594 (1986); and Sakmann et al., Annual Review of Physiology, 46:455-472 (1984).

Isolated hSlo2.2 proteins within the scope of this discovery include those which, when full-length and expressed in a cell from a quiet line, define a functionality and pharmacology indicative of a hSlo2.2 channel. A quiet line is a cell line that in its native state (e.g., not expressing hSlo2.2 channels) has low or uninteresting electric activity, e.g., a CHO cell line. For example, a control cell (without expression of a hSlo2.2 channel of the present invention) and an experimental cell (expressing a hSlo2.2 channel) are maintained under conditions standard for measurement of electrophysiological parameters as provided in the working examples disclosed herein.

Subsequently, measurements of the cells are taken to detect induction of ion flux (e.g., by radiotracer), or a change in ionic conductance of the cell (e.g., by patch clamp), or a change in voltage (e.g., by fluorescent dye). If the presence of an ion channel is indicated by a Na⁺ induced or voltage gated change, subsequent tests are used to characterize the channel as a hSlo2.2 channel of the present invention. For example, Na⁺ sensitivity can be determined as described above, using inside out patches in saline of different concentrations of Na⁺. Whole cell recordings in sodium bicarbonate or ammonium chloride can also be used. Compounds such as rotenone and FCCP can be used to alter the concentration of Na⁺ within the cell, and protonofors can also be used to determine Na⁺ sensitivity. Preferably, at least two characteristics are determined, more preferably at least 3, or 4 are determined. Characteristics of hSlo2.2 channels of the present invention are disclosed more fully herein.

For example, a cell expressing a hSlo2.2 channel of the present invention can have a conductance of between 80-120 pS, can comprise an hSlo2.2 channel protein monomer of about 120 to about 156 kD, can exhibit Na⁺ sensitivity (e.g., increased activity at above approximately Na⁺ concentration of 1 mm) and voltage-gating, can exhibit amino acid identity of at least 60%, and more preferably at least 70%, 80%, 90% or 95% in an alignment with the core domain of the exemplary mouse and hSlo2.2 channel sequences disclosed herein, and can be specifically reactive, under immunologically reactive conditions, with an antibody raised to an exemplary hSlo2.2 sequence disclosed herein (e.g., at least one of SEQ ID NO:1 and SEQ ID NO:2, and SEQ ID NO:3). Such standard methods aid in the identification of hSlo2.2 proteins of this invention.

Solid phase synthesis of hSlo2.2 channel polypeptides of less than about 50 amino acids in length may be accomplished by attaching the C-terminal amino acid of the sequence to an insoluble support followed by sequential addition of the remaining amino acids in the sequence useful. Techniques for solid phase synthesis are described by Merrifield et al., J. Am. Chem. Soc., 85:2149-2156 (1963), and Stewart et al., Solid Phase Peptide Synthesis, 2nd ed. Pierce Chem. Co., Rockford, Ill. (1984). Which is hereby incorporated herein in its entirety by reference. hSlo2.2 channel proteins of greater length may be synthesized by condensation of the amino and carboxy termini of shorter fragments. Methods of forming peptide bonds by activation of a carboxy terminal end (e.g., by the use of the coupling reagent N,N′-dicycylohexylcarbodiimide)) is known to those of skill.

General procedure for obtaining nucleic acids encoding Na⁺ sensitive potassium channel proteins

The present discovery provides isolated nucleic acids of RNA, DNA, and chimeras thereof, which encode hSlo2.2 channel proteins.

In an aspect, nucleic acids of the present invention are used as probes, for example, in detecting deficiencies in the level of mRNA, mutations in the gene (e.g., substitutions, deletions, or additions), for monitoring up regulation of hSlo2.2 channels in drug screening assays, or for recombinant expression of hSlo2.2 channel proteins for use as immunogens in the preparation of antibodies or for in vitro expression assays.

In an aspect, nucleic acids encoding the Na⁺ sensitive potassium channel proteins of the present invention are made using standard recombinant or synthetic techniques. Using the amino acid sequences of the hSlo2.2 channel proteins herein provided, one of skill in the art can after reading this specification readily construct a variety of clones containing functionally equivalent nucleic acids, such as nucleic acids that encode the same protein. Cloning methodologies to accomplish these ends, and sequencing methods to verify the sequence of nucleic acids are well known in the art. Examples of appropriate cloning and sequencing techniques, and instructions sufficient to direct persons of skill through many cloning exercises are found in Sambrook et al., Molecular Cloning: A Laboratory Manual (2d ed. 1989)); Methods in Enzymology, Guide to Molecular Cloning Techniques (Berger & Kimmel eds., 1987); and Current Protocols in Molecular Biology (Ausubel et al. eds., 1987). These referenced articles incorporated in their entirety by reference. Product information from manufacturers of biological reagents and experimental equipment also provide information useful in known biological methods. Such manufacturers include the SIGMA chemical company (Saint Louis, Mo.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRI Life Technologies, Inc. (Gaithersberg, Md.), Fluka Chemica-Biochemika Analytika (Fluka Chemie AG, Buchs, Switzerland), Invitrogen, San Diego, Calif., and Applied Biosystems (Foster City, Calif.), as well as many other commercial sources known to one of skill.

Isolation of hSlo2.2 Channel Proteins by Nucleic Acid Hybridization

In an aspect, the isolated nucleic acid compositions of this invention, whether RNA, cDNA, genomic DNA, or a hybrid of the various combinations, are isolated from biological sources or synthesized in vitro. Deoxynucleotides can be prepared by any suitable method including, for example, cloning and restriction of appropriate sequences or direct chemical synthesis by methods such as the phosphotriester method of Narang et al., Meth. Enzymol. 68:90-99 (1979); the phosphodiester method of Brown et al., Meth. Enzymol. 68:109-151 (1979); the diethylphosphoramidite method of Beaucage et al., Tetra. Lett., 22:1859-1862 (1981); the solid phase phosphoramidite triester method described by Beaucage & Caruthers, Tetrahedron Letts., 22:1859-1862 (1981), e.g., using an automated synthesizer, e.g., as described in Needham-VanDevanter et al., Nucleic Acids Res. 12:6159-6168 (1984); and the solid support method of U.S. Pat. No. 4,458,066. Chemical synthesis produces a single stranded oligonucleotide. This may be converted into double stranded DNA by hybridization with a complementary sequence, or by polymerization with a DNA polymerase using the single strand as a template. One of skill would recognize that while chemical synthesis of DNA is limited to sequences of about 100 bases, longer sequences may be obtained by the ligation of shorter sequences.

Using the hSlo2.2 nucleic acids as a probe (includes all numbered hSlo2.2 nucleic acid sequences), a human cDNA library is probed using moderate stringency conditions to isolate an hhSlo2.2 clone. Alternatively, amplification primers for hSlo2.2 can be designed that correspond to the conserved core domain of hSlo2.2. These primers are used to amplify cDNA corresponding to hSlo2.2.

The isolated nucleic acids of the present invention may be cloned, or amplified from brain or heart mRNA or cDNA by in vitro methods, such as the polymerase chain reaction (PCR), the ligase chain reaction (LCR), and the transcription-based amplification system (TAS). Alternatively, the isolated hSlo2.2 nucleic acids of the present invention can be isolated by screening brain or heart, kidney or testis cDNA or genomic libraries with a hSlo2.2 probe. Moreover, a brain or heart, kidney or testis expression library can be screening with an antibody that binds to hSlo2.2. A wide variety of cloning and in vitro amplification methodologies are well-known to persons of skill. Examples of these techniques and instructions sufficient to direct persons of skill through many cloning exercises are found in Sambrook et al. supra, and Ausubel et al., supra.

Examples of useful techniques sufficient to direct persons of skill through in vitro amplification methods are found in Sambrook and Ausubel, as well as Mullis et al., U.S. Pat. No. 4,683,202; PCR Protocols A Guide to Methods and Applications (Innis et al. eds, 1990); The Journal Of NIH Research 3:81-94 (1991); Kwoh et al., Proc. Natl. Acad. Sci. USA 86:1173 (1989); Guatelli et al., Proc. Natl. Acad. Sci. USA 87:1874 (1990); Lomell et al., J. Clin. Chem. 35:1826 (1989); Landegren et al., Science 241:1077-1080 (1988); Van Brunt, Biotechnology 8:291-294 (1990); Wu & Wallace, Gene 4:560 (1989); and Barringer et al., Gene 89:117 (1990).

Isolated nucleic acids encoding hSlo2.2 channel proteins comprise a nucleic acid sequence encoding a hSlo2.2 channel protein selected from the group consisting of at least one of SEQ ID NO:1 and SEQ ID NO:2 and subsequences thereof.

In addition to the isolated nucleic acids identified herein, this discovery includes other isolated nucleic acids encoding Na⁺ sensitive potassium channel proteins which selectively hybridize, under stringent conditions, to a nucleic acid encoding a protein selected from the group consisting of at least one of SEQ ID NO:1 and SEQ ID NO:2, and subsequences thereof. Generally, the isolated nucleic acid encoding a Na⁺ sensitive potassium channel protein of the present invention will hybridize under at least moderate stringency hybridization conditions to a nucleic acid sequence from at least one of SEQ ID NOS:1 and 2, which encodes the core domain or subsequence thereof. Alternatively, or additionally, the isolated nucleic acid encoding the Na⁺ sensitive potassium channel protein will encode an amino acid sequence of at least 60%, 70%, 80%, or 90% similarity over the length of the core domain. Conveniently, the nucleic acid encoding a subsequence of the core region is obtained from at least one of SEQ ID NOS:1 and 2, and is at least any one of from 15 to 1800 nucleotides in length, and generally at least 200 or 600 nucleotides in length; preferably the nucleic acid will encode the entire core sequence. The nucleic acid sequence, or subsequence thereof, encoding the Na⁺ sensitive potassium channel protein comprises at least N′ nucleotides in length, where N′ is any one of the integers selected from the group consisting of from 18 to 5000. Thus, the nucleic acids of the present invention comprise genomic DNA and nuclear transcripts encoding hSlo2.2 channel proteins.

In aspects where the nucleic acid encoding a hSlo2.2 channel protein is used as nucleic acid probes, it is often desirable to label the nucleic acid with detectable labels. The labels may be incorporated by any of a number of means well known to those of skill in the art. However, in a preferred embodiment, the label is simultaneously incorporated during the amplification step in the preparation of the nucleic acids. Thus, for example, polymerase chain reaction (PCR) with labeled primers or labeled nucleotides will provide a labeled amplification product. In another preferred embodiment, transcription amplification using a labeled nucleotide (e.g., fluorescein-labeled UTP and/or CTP) incorporates a label into the transcribed nucleic acids.

Alternatively, a label may be added directly to an original nucleic acid sample (e.g., mRNA, poly A⁺ mRNA, cDNA, etc.) or to the amplification product after the amplification is completed. Means of attaching labels to nucleic acids are well known to those of skill in the art and include, for example, nick translation or end-labeling (e.g., with a labeled RNA) by phosphorylation of the nucleic acid and subsequent attachment (ligation) of a nucleic acid linker joining the sample nucleic acid to a label (e.g., a fluorophore).

Detectable labels suitable for use in the present invention include any composition detectable by spectroscopic, radioisotopic, photochemical, biochemical, immunochemical, electrical, optical or chemical means. Useful labels in the present invention include biotin for staining with labeled streptavidin conjugate, magnetic beads, fluorescent dyes (e.g., fluorescein, texas red, rhodamine, green fluorescent protein, and the like), radiolabels (e.g., ³H, ¹²⁵l ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. U.S. patents teaching the use of such labels include U.S. Pat. Nos. 3,817,837; 3,850,752; 3,939,350; 3,996,345; 4,277,437; 4,275,149; and 4,366,241.

Means of detecting such labels are well known to those of skill in the art. Thus, for example, radiolabels may be detected using photographic film or scintillation counters, and fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The probes are used to screen genomic or cDNA libraries from any source of interest including specific tissues (e.g., brain or heart and other tissues of interest such as kidney tissue) and animal sources such as rat, human, bird, etc. Screening techniques are known in the art and are described in the general texts cited above such as in Sambrook and Ausubel, supra.

Isolation of hSlo2.2 Channel Proteins by Immunoscreening

In addition to using nucleic acid probes for identifying homologues and family members of hSlo2.2, it is possible to use antibodies to probe expression libraries. This is a well known technology (see, e.g. Young & Davis, Proc. Natl. Acad. Sci., U.S.A. 80:1194-1198 (1982)). In general, a cDNA expression library maybe prepared from commercially available kits or using readily available components. Phage vectors are preferred, but a variety of other vectors are available for the expression of protein. Such vectors are those that can be used for expression in, e.g., yeast, animal cells and Xenopus oocytes. One selects mRNA from a source that is enriched with the target protein, e.g., brain or heart, and creates cDNA which is then ligated into a vector and transformed into the library host cells for immunoscreening. Screening involves binding and visualization of antibodies bound to specific proteins on cells or immobilized on a solid support such as nitrocellulose or nylon membranes. Positive clones are selected for purification to homogeneity and the isolated cDNA then prepared for expression in the desired host cells. When choosing to obtain Na⁺ sensitive channel proteins, antibodies selective for the entire protein or portions of the protein can be used, e.g., the core domain.

Nucleic Acid Assays

In an aspect, methods of detecting and/or quantifying hSlo2.2 channel protein expression by assaying for the gene transcript (e.g., nuclear RNA, mRNA) are provided. The assay can be for the presence or absence of the normal gene or gene product, for the presence or absence of an abnormal gene or gene product, or quantification of the transcription levels of normal or abnormal hSlo2.2 channel protein gene product.

In an aspect, nucleic acid assays are performed with a sample of nucleic acid isolated from the organism to be tested. In the simplest embodiment, such a nucleic acid sample is the total mRNA isolated from a biological sample. The nucleic acid (e.g., either genomic DNA or mRNA) may be isolated from the sample according to any of a number of methods well known to those of skill in the art.

Methods of isolating total DNA or mRNA are well known to those of skill in the art. For example, methods of isolation and purification of nucleic acids are described in detail in Chapter 3 of Laboratory Techniques in Biochemistry and Molecular Biology: Hybridization With Nucleic Acid Probes, Part L Theory and Nucleic Acid Preparation (Tijssen, ed. 1993). One of skill in the art will appreciate that where alterations in the copy number of the gene encoding hSlo2.2 channel protein is to be detected genomic DNA is preferably isolated. Conversely, where expression levels of a gene or genes are to be detected, preferably RNA (mRNA) is isolated.

Frequently, it is desirable to amplify the nucleic acid sample prior to hybridization. One of skill in the art will appreciate that whatever amplification method is used, if a quantitative result is desired, care must be taken to use a method that maintains or controls for the relative frequencies of the amplified nucleic acids. Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The high density array may then include probes specific to the internal standard for quantification of the amplified nucleic acid. Detailed protocols for quantitative PCR are provided in PCR Protocols, A Guide to Methods and Applications (Innis et al., ed. 1990).

In an aspect, a method of detecting the nucleic acid sequence encoding a hSlo2.2 channel protein comprises: (a) contacting the biological sample, under stringent hybridization conditions with a nucleic acid probe comprising a nucleic acid segment which selectively hybridizes to a nucleic acid sequence (target) encoding hSlo2.2 channel protein selected from the group consisting of at least one of SEQ ID NO:1, and allowing the probe to specifically hybridize to the nucleic acid encoding hSlo2.2 channel protein to form a hybridization complex, wherein detection of the hybridization complex is an indication of the presence of the hSlo2.2 nucleic acid sequence in the sample. The nucleic acid segment of the probe is a subsequence of at least N″ contiguous nucleotides in length from a nucleic acid encoding a hSlo2.2 channel selected from the group consisting of at least one of SEQ ID NO:1 and SEQ ID NO:2, and complementary sequences thereof, where N″ is an any one of the integers selected from the group consisting of each of the integers from 15 to 1500.

As used herein, contiguous nucleotides” from a referenced nucleic acid means a sequence of nucleotides having the same order and directly adjacent to the same nucleotides (i.e., without additions or deletions) as in the referenced nucleic acid. Typically, the nucleic acid segment is at least 18 nucleotides in length. The preferred length of the nucleic acid probe is from 24 to 200 nucleotides in length. In particularly preferred embodiments, the nucleic acid segment is derived from a nucleic acid which encodes a core region from a protein selected from the group consisting of at least one of SEQ ID NO:1 and SEQ ID NO:2. Usually, and particularly for cross-species hybridization, the nucleic acid segment would encode an amino acid sequence from within the core region and will be at least 250 nucleotides in length, most preferably will encode the entirety of the core region, and/or will hybridize to the target sequence under moderate stringency hybridization conditions.

Those of skill in the art will appreciate that nucleic acid sequences of the probe will be selected so as not to interfere in the selective hybridization of the nucleic acid segment to the target. Thus, for example, any additional nucleotides attached to the nucleic acid segment will generally be chosen so as not to selectively hybridize, under stringent conditions, to the nucleic acid target (potential false negative), nor to nucleic acids not encoding a hSlo2.2 channel protein or peptide (potential false positive). The use of negative and positive controls to ensure selectivity and specificity is known to those of skill. In general, the length of the probe should be kept to the minimum length necessary to achieve the desired results. The length of the nucleic acid encoding a hSlo2.2 channel protein or peptide is discussed more fully, supra, but is preferably at least 30 nucleotides in length.

A variety of nucleic acid hybridization formats are known to those skilled in the art. For example, common formats include sandwich assays and competition or displacement assays. Hybridization techniques are generally described in Berger & Kimmel, supra; Gall & Pardue, Proc. Natl. Acad. Sci., U.S.A. 63:378-383 (1969); and John et al., Nature 223:582-587 (1969)).

Sandwich assays are commercially useful hybridization assays for detecting or isolating nucleic acid sequences. Such assays utilize a “capture” nucleic acid covalently immobilized to a solid support and a labelled “signal” nucleic acid in solution. The biological sample will provide the target nucleic acid. The “capture” nucleic acid probe and the “signal” nucleic acid probe hybridize with the target nucleic acid to form a sandwich” hybridization complex. To be effective, the signal nucleic acid cannot hybridize with the capture nucleic acid.

In in situ hybridization, the target nucleic acid is liberated from its cellular surroundings in such as to be available for hybridization within the cell while preserving the cellular morphology for subsequent interpretation and analysis. The following articles provide an overview of the art of in situ hybridization: Singer et al., Biotechniques 4:230-250 (1986); Haase et al., Methods in Virology, Vol. VII, pp. 189-226 (1984); and Nucleic Acid Hybridization: A Practical Approach (Hames et al., eds. 1987).

Typically, labeled signal nucleic acids are used to detect hybridization. Complementary nucleic acids or signal nucleic acids may be labelled by any one of several methods typically used to detect the presence of hybridized oligonucleotides. The most common method of detection is the use of autoradiography with ³H, ¹²⁵I ³⁵S, ¹⁴C, or ³²P-labelled probes and the like. Other useful labels include ligands that bind to labeled antibodies, fluorophores, chemiluminescent agents, enzymes, and antibodies that can serve as specific binding pair members for a labelled ligand.

The label may also allow for the indirect detection of the hybridization complex. For example, where the label is a hapten or antigen, the sample can be detected by using antibodies. In these systems, a signal is generated by attaching fluorescent or enzyme molecules to the antibodies or, in some cases, by attachment to a radioactive label (Tijssen, Practice and Theory of Enzyme Immunoassays, in Laboratory Techniques in Bio-chemistry and Molecular Biology, pp 9-20 (1985)).

The detectable label used in nucleic acids of the present invention may be incorporated by any of a number of means known to those of skill in the art, e.g., as discussed supra. Means of detecting such labels are well known to those of skill in the art.

Those of skill in the art will appreciate that abnormal expression levels or abnormal expression products (e.g., mutated transcripts, truncated or non-sense proteins) are identified by comparison to normal expression levels and normal expression products. Normal levels of expression or normal expression products can be determined for any particular population, subpopulation, or group of organisms according to standard methods known to those of skill in the art. Typically this involves identifying healthy organisms (i.e., organisms with a functional hSlo2.2 channel protein as indicated by such properties as conductance and Na⁺ sensitivity) and measuring expression levels of the hSlo2.2 channel protein gene (as described herein) or sequencing the gene, mRNA, or reverse transcribed cDNA, to obtain typical (normal) sequence variations. Application of standard statistical methods used in molecular genetics permits determination of baseline levels of expression, and normal gene products as well as significant deviations from such baseline levels.

In an aspect, nucleic acids of this invention are included in a kit used to determine in a biological sample the presence or absence of the normal gene or gene product encoding a hSlo2.2 channel of the present invention, for the presence or absence of an abnormal gene or gene product encoding a hSlo2.2 channel, or quantification of the transcription levels of normal or abnormal hSlo2.2 channel protein gene product. The kit typically includes a stable preparation of nucleic acid probes for performing the assay of the present invention. Further, the kit may also include a hybridization solution in either dry or liquid form for the hybridization of probes to target Na⁺ sensitive potassium channel proteins or Na⁺ sensitive potassium channel protein nucleic acids of the present invention, a solution for washing and removing undesirable and non-hybridized nucleic acids, a substrate for detecting the hybridization complex, and/or instructions for performing and interpreting the assay.

Once the nucleic acids encoding a hSlo2.2 channel protein of the present invention are isolated and cloned, one may express the desired protein in a recombinantly engineered cell such as Xenopus, bacteria, yeast, insect (especially employing baculoviral vectors), and mammalian cells.

After reading this specification, those of skill in the art will be able to use expression systems available for expression of DNA encoding a hSlo2.2 channel protein(s).

In an aspect, the expression of natural or synthetic nucleic acids encoding Na⁺ sensitive potassium channel proteins of the present invention is achieved by operably linking the DNA or cDNA to a suitable promoter (which is either constitutive or inducible), followed by competent stable incorporation into an expression vector. The vectors are suitable for replication and integration in either prokaryotes or eukaryotes. Typical useful expression vector(s) contain transcription and translation terminators, initiation sequences, and promoters useful for regulation of the expression of the DNA encoding the hSlo2.2 channel protein. To obtain high level expression of a cloned gene, it is desirable to construct expression vectors which contain, at the minimum, a strong promoter to direct transcription, a ribosome binding site for translational initiation, and a transcription/translation terminator. After reading this specificity one of skill would recognize that modifications can be made to a hSlo2.2 channel protein without diminishing its biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

Suitable bacterial promoters are well known in the art and described, e.g., in Sambrook et al. and Ausubel et al. Bacterial expression systems for expressing the hSlo2.2 protein are available in, e.g., E. coli, Bacillus sp., and Salmonella (Palva et al., Gene 22:229-235 (1983); Mosbach et al., Nature, 302:543-545 (1983). Kits for such expression systems are commercially available. Eukaryotic expression systems for mammalian cells, yeast, and insect cells are well known in the art and are also commercially available.

A “promoter” is defined as an array of nucleic acid control sequences that direct suitable transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements that can be located as much as several thousand base pairs from the start site of transcription. A “constitutive” promoter is a promoter that is active under most environmental and developmental conditions. An “inducible” promoter is a promoter that is under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid.

The promoter used to direct expression of a heterologous nucleic acid depends upon the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the hSlo2.2 encoding DNA in host cells. A typical expression cassette thus contains a promoter operably linked to the DNA sequence encoding hSlo2.2 and signals required for efficient polyadenylation of the transcript, ribosome binding sites, and translation termination. The DNA sequence encoding the hSlo2.2 may typically be linked to a cleavable signal peptide sequence to promote secretion of the encoded protein by the transformed cell. Such signal peptides would include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, and fusion expression systems such as GST and LacZ. Epitope tags can also be added to recombinant proteins to provide convenient methods of isolation.

Useful expression vectors containing regulatory elements from eukaryotic viruses are typically used in eukaryotic expression vectors, e.g., SV40 vectors, papilloma virus vectors, and vectors derived from Epstein Bar virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as using a baculovirus vector in insect cells, with a hSlo2.2 encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, as any of the many resistance genes known in the art are suitable. The prokaryotic sequences are preferably chosen such that they do not interfere with the replication of the DNA in eukaryotic cells, if necessary.

Mammalian cell systems often will be in the form of monolayers of cells although mammalian cell suspensions may also be used. A number of suitable host cell lines capable of expressing intact proteins have been developed in the art, and include the HEK293, BHK21, and CHO cell lines, and various human cells such as COS cell lines, HeLa cells, myeloma cell lines, Jurkat cells. In some embodiments, Xenopus oocytes are used. Those of skill will recognize that preferred cell lines for expressing hSlo2.2 channels substantially lack conductances which compete with those provided by the Na⁺ sensitive potassium channels of the present invention (i.e., “quiet lines”). Other animal cells useful for production of hSlo2.2 channel proteins are available, for instance, from the American Type Culture Collection Catalogue of Cell Lines and Hybridomas (7th ed. 1992).

Standard transfection methods are used to produce bacterial, mammalian, yeast or insect cell lines that express large quantities of hSlo2.2 protein, which are then purified using standard techniques (see, e.g., Colley et al., .J. Biol. Chem. 264:17619-17622 (1989); Guide to Protein Purification, in Methods in Enzymology, vol. 182 (Deutscher ed., 1990)). Transformation of eukaryotic and prokaryotic cells are performed according to standard techniques (see, e.g., Morrison, J. Bact., 132:349-351 (1977); Clark-Curtiss & Curtiss, Methods in Enzymology, 101:347-362 (Wu et al., eds, 1983)).

In an aspect, well known procedures for introducing foreign nucleotide sequences into host cells are used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g. Sambrook et al., supra). It is only necessary that the particular genetic engineering procedure used be capable of successfully introducing at least one gene into the host cell capable of expressing the hSlo2.2 protein.

After the expression vector is introduced into the cells, the transfected cells are cultured under conditions favoring expression of the hSlo2.2 protein which is recovered from the culture using standard techniques identified below.

Alternatively, hSlo2.2 protein can be expressed transiently in a cell by introducing into a cell an RNA encoding the hSlo2.2 protein. In an aspect, the RNA is transcribed in vitro according to standard procedures and then introduced into a cell by means such as injection or electroporation. The RNA then expresses the hSlo2.2 protein. Such systems are useful for measuring single channel and whole cell conductance of a hSlo2.2 channel protein, e.g., when the RNA is transiently expressed in cells such as Xenopus oocytes, CHO, and HeLa cells.

Purification of hSlo2.2 Protein

Either naturally occurring or recombinant hSlo2.2 can be purified for use in functional assays. Naturally occurring hSlo2.2 is purified, e.g., from brain, kidney, or heart tissue. Recombinant hSlo2.2 is purified from any suitable expression system as illustrated below in nonlimiting fashion.

In an aspect, hSlo2.2 is purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed when recombinant hSlo2.2 is being purified. For example, proteins having established molecular adhesion properties can be reversible fused to hSlo2.2. With the appropriate ligand, hSlo2.2 can be selectively adsorbed to a purification column and then freed from the column in a relatively pure form. The fused protein is then removed by enzymatic activity. Finally, hSlo2.2 could be purified using immunoaffinity columns.

Purification of hSlo2.2 from Recombinant Bacteria

Recombinant proteins are expressed by transformed bacteria in large amounts, typically after promoter induction; but expression can be constitutive. Bacteria are grown according to standard procedures in the art.

Proteins expressed in bacteria may form insoluble aggregates (“inclusion bodies”). Several protocols are suitable for purification of hSlo2.2 inclusion bodies. For example, purification of inclusion bodies typically involves the extraction, separation and/or purification of inclusion bodies by disruption of bacterial cells, e.g., by incubation in a buffer of about 100-150 μg/ml lysozyme and 0.1% Nonidet P40, a non-ionic detergent. The cell suspension can be homogenized using a Polytron (Brinkman Instruments, Westbury, N.Y.). Alternatively, the cells can be sonicated on ice. Alternate methods of lysing bacteria are apparent to those of skill in the art (see, e.g., Sambrook et al., supra; Ausubel et al., supra).

The cell suspension is generally centrifuged and the pellet containing the inclusion bodies resuspended in buffer that does not dissolve but washes the inclusion bodies, e.g., 20 mM Tris-HCl (pH 7.2), 1 mM EDTA, 150 mM NaCI and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCI). Other appropriate buffers will be apparent to those of skill in the art after reading this specification.

Following washing, the inclusion bodies are solubilized by the addition of a solvent that is both a strong hydrogen acceptor and a strong hydrogen donor (or a combination of solvents each having one of these properties); the proteins that formed the inclusion bodies may then be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of immunologically and/or biologically active protein. After solubilization, the protein can be separated from other bacterial proteins by standard separation techniques.

Alternatively, it is possible to purify hSlo2.2 from bacteria periplasm. Where hSlo2.2 is exported into the periplasm of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

Standard Protein Separation Techniques for Purifying hSlo2.2

Solubility Fractionation

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

Size Differential Filtration

hSlo2.2 has a known molecular weight and this knowledge can be used to isolated it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

Column Chromatography

In an aspect, hSlo2.2 is be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

Immunological Detection of hSlo2.2

In addition to the detection of hSlo2.2 genes and gene expression using nucleic acid hybridization technology, one can also use immunoassays to detect hSlo2.2. Immunoassays can be used to qualitatively or quantitatively analyze hSlo2.2. A general overview of the applicable assay technology is found in Harlow & Lane, Antibodies: A Laboratory Manual (1988).

Antibodies to hSlo2.2

Methods of producing polyclonal and monoclonal antibodies that react specifically with hSlo2.2 are known to those of skill in the art (see, e.g., Coligan, Current Protocols in Immunology (1991); Harlow & Lane, supra; Goding, Monoclonal Antibodies: Principles and Practice (2d ed. 1986); and Kohier & Milstein, Nature, 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g, Huse et al., Science 246:1275-1281 (1989); Ward et al., Nature 341:544-546 (1989)).

A number of hSlo2.2 comprising immunogens may be used to produce antibodies specifically reactive with hSlo2.2. For example, recombinant hSlo2.2 or a antigenic fragment thereof such as the core or tail domain, is isolated as described herein. Recombinant protein can be expressed in eukaryotic or prokaryotic cells as described above, and purified as generally described above. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. An inbred strain of mice or rabbits is immunized with the protein using a standard adjuvant, such as Freund's adjuvant, and a standard immunization protocol. The animal's immune response to the immunogen preparation is monitored by taking test bleeds and determining the titer of reactivity to hSlo2.2. When appropriately high titers of antibody to the immunogen are obtained, blood is collected from the animal and antisera are prepared. Further fractionation of the antisera to enrich for antibodies reactive to the protein can be done if desired (see Harlow & Lane, supra).

Monoclonal antibodies may be obtained by various techniques familiar to those skilled in the art. Briefly, spleen cells from an animal immunized with a desired antigen are immortalized, commonly by fusion with a myeloma cell (see Kohier & Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse et al., Science 246:1275-1281 (1989).

Monoclonal antibodies and polyclonal sera are collected and titered against the immunogen protein in an immunoassay, for example, a solid phase immunoassay with the immunogen immobilized on a solid support. Polyclonal antisera with a titer of 10⁴ or greater are selected and tested for their cross reactivity against non-hSlo2.2 proteins or even other homologous proteins from other organisms, using a competitive binding immunoassay. Specific polyclonal antisera and monoclonal antibodies will usually bind with a K_{D} of at least about 0.1 mM, more usually at least about 1 μM, preferably at least about 0.1 μM or better, and most preferably, 0.01 μM or better.

Once hSlo2.2 specific antibodies are available, hSlo2.2 can be detected by a variety of immunoassay methods. For a review of immunological and immunoassay procedures, see Basic and Clinical Immunology (Stites & Terr eds., 7th ed. 1991). Moreover, the immunoassays of the present invention can be performed in any of several configurations, which are reviewed extensively in Enzyme Immunoassay (Maggio, ed., 1980); and Harlow & Lane, supra.

Immunological Binding Assays

As explained above, hSlo2.2 expression is associated with sperm physiology, e.g., capacitation and acrosome reactions. Thus, hSlo2.2 provides a marker with which to examine these reactions in sperm. In a preferred embodiment, hSlo2.2 is detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology Volume 37: Antibodies in Cell Biology (Asai, ed. 1993); Basic and Clinical Immunology (Stites & Terr, eds., 7th ed. 1991). Immunological binding assays (or immunoassays) typically utilize a “capture agent” to specifically bind to and often immobilize the analyte (in this case the hSlo2.2 or antigenic subsequence thereof). The capture agent is a moiety that specifically binds to the analyte. The antibody (anti-hSlo2.2) may be produced by any of a number of means well known to those of skill in the art and as described above.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled hSlo2.2 polypeptide or a labeled anti-hSlo2.2 antibody. Alternatively, the labeling agent may be a third moiety, such as another antibody, that specifically binds to the antibody/hSlo2.2 complex.

In an aspect, the labeling agent is a second hSlo2.2 bearing a label. Alternatively, the second antibody may lack a label, but it may, in turn, be bound by a labeled third antibody specific to antibodies of the species from which the second antibody is derived. The second can be modified with a detectable moiety, such as biotin, to which a third labeled molecule can specifically bind, such as enzyme-labeled streptavidin.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see generally Kronval, et al., J. Immunol., 111: 1401-1406 (1973); Akerstrom, et al., J. Immunol., 135: 2589-2542 (1985)).

Throughout the assays, incubation and/or washing steps may be required after each combination of reagents. Incubation steps can vary from about 5 seconds to several hours, preferably from about 5 minutes to about 24 hours. However, the incubation time will depend upon the assay format, analyte, volume of solution, concentrations, and the like. Usually, the assays will be carried out at ambient temperature, although they can be conducted over a range of temperatures, such as 10° C. to 40° C.

Non-Competitive Assay Formats

Immunoassays for detecting hSlo2.2 in samples may be either competitive or noncompetitive. Noncompetitive immunoassays are assays in which the amount of captured analyte (in this case the protein) is directly measured. In one preferred “sandwich” assay, for example, the capture agent (anti-hSlo2.2 antibodies) can be bound directly to a solid substrate on which they are immobilized. These immobilized antibodies then capture hSlo2.2 present in the test sample. hSlo2.2 is thus immobilized is then bound by a labeling agent, such as a second hSlo2.2 antibody bearing a label. Alternatively, the second antibody may lack a label, but it may, in turn, be bound by a labeled third antibody specific to antibodies of the species from which the second antibody is derived. The second can be modified with a detectable moiety, such as biotin, to which a third labeled molecule can specifically bind, such as enzyme-labeled streptavidin.

Competitive Assay Formats

In competitive assays, the amount of hSlo2.2 (analyte) present in the sample is measured indirectly by measuring the amount of an added (exogenous) analyte (i.e, the hSlo2.2) displaced (or competed away) from a capture agent (anti-hSlo2.2 antibody) by the analyte present in the sample. In one competitive assay, a known amount of, in this case, the hSlo2.2 is added to the sample and the sample is then contacted with a capture agent, in this case an antibody that specifically binds to the hSlo2.2. The amount of hSlo2.2 bound to the antibody is inversely proportional to the concentration of hSlo2.2 present in the sample. In a particularly preferred embodiment, the antibody is immobilized on a solid substrate. The amount of the hSlo2.2 bound to the antibody may be determined either by measuring the amount of hSlo2.2 present in a hSlo2.2/antibody complex, or alternatively by measuring the amount of remaining uncomplexed protein. The amount of hSlo2.2 may be detected by providing a labeled hSlo2.2 molecule.

A hapten inhibition assay is another competitive assay. In this assay a known analyte, in this case hSlo2.2, is immobilized on a solid substrate. A known amount of anti-hSlo2.2 antibody is added to the sample, and the sample is then contacted with the immobilized hSlo2.2. The amount of anti-hSlo2.2 antibody bound to the immobilized hSlo2.2 is inversely proportional to the amount of hSlo2.2 present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

Immunoassays in the competitive binding format can be used for crossreactivity determinations. For example, a protein partially encoded by at least one of SEQ ID NO:1 or SEQ ID NO:2, can be immobilized to a solid support. Proteins are added to the assay that compete with the binding of the antisera to the immobilized antigen. The ability of the above proteins to compete with the binding of the antisera to the immobilized protein is compared to hSlo2.2 encoded by at least one of SEQ ID NO:1 or SEQ ID NO:2. The percent crossreactivity for the above proteins is calculated, using standard calculations. Those antisera with less than 10% crossreactivity with each of the proteins listed above are selected and pooled. The cross-reacting antibodies are optionally removed from the pooled antisera by immunoabsorption with the considered proteins, e.g., distantly related homologues.

The immunoabsorbed and pooled antisera are then used in a competitive binding immunoassay as described above to compare a second protein, thought to be perhaps the protein of this invention, to the imniunogen protein (i.e., hSlo2.2 comprising at least one of SEQ ID NO:1 or SEQ ID NO:2). In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the protein partially encoded by at least one of SEQ ID NO:1 or SEQ ID NO:2, that is required to inhibit 50% of binding, then the second protein is said to specifically bind to the polyclonal antibodies generated to a hSlo2.2 immunogen.

Other Useful Assay Formats

Western blot (immunoblot) analysis is used to detect and quantify the presence of hSlo2.2 in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the hSlo2.2. The anti-hSlo2.2 antibodies specifically bind to the hSlo2.2 on the solid support. These antibodies may be directly labeled or alternatively may be subsequently detected using labeled antibodies (e.g., labeled sheep anti-mouse antibodies) that specifically bind to the anti-hSlo2.2 antibodies.

Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev. 5:34-41 (1986)).

Reduction of Non-Specific Binding

One of skill in the art will appreciate that it is often desirable to minimize non-specific binding in immunoassays. Particularly, where the assay involves an antigen or antibody immobilized on a solid substrate it is desirable to minimize the amount of nonspecific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

Labels

The particular label or detectable group used in the assay is not a critical aspect of the discovery, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., Dynabeads™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand-then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used in conjunction with the labeled, naturally occurring anti-ligands. Alternatively, any haptenic or antigenic compound can be used in combination with an antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Illustrative Chemiluminescent compounds include luciferin, and 2,3-dihydrophthalazinediones, e.g., luminol. For a review of various labeling or signal producing systems which may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

Isolated hSlo2.2 channel nucleic acids which are expressed in cells can be used in a variety of assays to identify compounds that modulate (increase or decrease) the flux (i.e., influx or efflux) of ions such as potassium, sodium, and potassium and analogues such as rubidium through the hSlo2.2 channels, respectively.

In an aspect, output of an assay coupled to a frengenic cell, is utilize to evaluate and determine and classify the therapeutic value of molecules to add to a transgenic cell expressing one of rSLO2 to hSLO2.2. The assig output of different externally presented modules 1 to the transgenic cell) is expected to classify, judge or determine which such modules are to be classified as therapeutic. In such a system these molecules determine to be therapeutic may be selected by more screening, based on an advanced screening track or dropped/discontinued in a screening program.

This use provides one advantageous method of screening libraries of compounds and moieties to identify those having therapeutic value.

Such determinates and classifications provided compared library management guidelines and techniques for such model systems.

Generally, compounds that decrease ion flux will cause a decrease by at least 10% as compared to a control or baseline, most preferably by at least 50% or 75% and higher. Compounds that increase the flux of ions will cause a detectable increase in the ion current density by increasing the probability of a hSlo2.2 channel being open, by decreasing the probability of it being closed, increasing conductance through the channel, and allowing the passage of ions. Typically the flux will increase by at least 10% as compared to a baseline control, more preferably 100%, often by at least 400%, 600%, 1,000%, 5,000% or 10,000%.

In an aspect modulation (increased or decreased) flux of ions may be assessed by determining changes in polarization (i.e., electrical potential) of the cell expressing the hSlo2.2 channel. A means to determine changes in cellular polarization is the voltage-clamp technique. Whole cell currents are conveniently determined using the conditions set forth in Example IV. Other known assays include: radiolabeled rubidium flux assays and fluorescence assays using voltage-sensitive dyes (see, e.g., Vestergarrd-Bogind et al., J. Membrane Biol. 88:67-75 (1988); Daniel et al., J. Pharmacol. Meth. 25:185-193 (1991); Holevinsky et al., J Membrane Biology 137:59-70 (1994)). Assays for compounds capable of inhibiting or increasing potassium flux through the hSlo2.2 channel protein can be performed by application of the compounds to a bath solution in contact with and comprising cells having an hSlo2.2 channel of the present invention (see, e.g., Blatz et al., Nature 323:718-720 (1986); Park, J. Physiol. 481:555-570 (1994)). Generally, the compounds to be tested are present in the range from about 1 pM to about 100 mM. Changes in function of the channels can be measured in the electrical currents or ionic flux, or by the consequences of changes in currents and flux.

The effects of test compounds upon the function of the channels can be measured by changes in the electrical currents or ionic flux or by the consequences of changes in currents and flux. Changes in electrical current or ionic flux are measured by either increases or decreases in flux of cations such as potassium or rubidium ions. The cations can be measured in a variety of standard ways. They can be measured directly by concentration changes of the ions or indirectly by membrane potential or by radiolabeling of the ions. Consequences of the test compound on ion flux can be quite varied. Accordingly, any suitable physiological change can be used to assess the influence of a test compound on the channels of this invention. The effects of a test compound can be measured by a toxin binding assay. When the functional consequences are determined using intact cells or animals, one can also measure a variety of effects such as transmitter release (e.g., dopamine), hormone release (e.g., insulin), transcriptional changes to both known and uncharacterized genetic markers (e.g., northern blots), cell volume changes (e.g., in red blood cells), immuno-responses (e.g., T cell activation), changes in cell metabolism such as cell growth or Na⁺ changes, and changes in intracellular second messangers such as Cl⁻.

Preferably, the hSlo2.2 channel of the assay will be selected from a channel protein of at least one of SEQ ID NO:1 or SEQ ID NO:2, conservatively modified variant thereof. Alternatively, the hSlo2.2 channel of the assay will be derived from a eukaryote and include an amino acid subsequence having sequence similarity to the core domain (S0-S8 of hSlo2.2) hSlo2.2 and hSlo2.2 channel proteins. Generally, the functional hSlo2.2 channel protein will be at least 200-600 amino acids in length. Generally, the sequence similarity will be at least 60%, typically at least 70%, generally at least 75%, preferably at least 80%, more preferably at least 85%, most preferably at least 90%, and often at least 95%. Thus, hSlo2.2 channel homologues will hybridize, under at least moderate hybridization conditions, to a nucleic acid of at least 100 nucleotides in length from the core domain of an hSlo2.2 nucleic acid and complementary sequences thereof.

The hSlo2.2 channel homologues will generally have substantially similar conductance characteristics (e.g., 80-120 pS) and Na⁺ sensitivity characteristics, as described above. Chimeras formed by expression of at least two hSlo2.2 may also be used. In a preferred embodiment, the cell placed in contact with a compound which is assayed for increasing or decreasing ion flux is a eukaryotic cell, e.g., an oocyte of Xenopus (e.g., Xenopus laevis) or a mammalian cell such as a CHO or HeLa cell.

hSlo2.2 is an active gene that is likely to encode an ion channel protein that can participate in disease processes of the type known to involve the potassium ion channels. It may be advantageous to modulate the K ion channel activity to treat such a disease state, either by increasing or decreasing the activity. This can be accomplished by targeting pharmaceuticals to the protein encoded by K. It will also be necessary to consider the physiological effect on those gene products on any treatment that modulates the activity of potassium ion channels.

Useful non-limiting examples of an express system utilizing hSlo2 and hSlo2.2 include a vector construct engineered for cRNA production which is used for injection of cRNA in amphibian oocyte expression system and a b. a vector engineered for expression in a mamalian, vertebrate, or invertebrate cell line. Such vector will use appropriate promoters for gene expression, such as a viral promoter for expression mammalian cell lines. (B) can be used for: 1. Transient expression assay and/or 2. Creation of a stabile transfected cell line in a mammalian, vertebrae, or invertebrate cell type.

Computer Assisted Drug Design

In an aspect, an assay for compounds that modulate potassium flux in Na⁺ sensitive potassium channels involves computer assisted drug design, in which a computer system is used to generate a three-dimensional structure of hSlo2.2 proteins based on the structural information encoded by the amino acid sequence. The amino acid sequence interacts directly and actively with a preestablished algorithm in a computer program to yield secondary, tertiary, and quaternary structural models of the protein. The models of the protein structure are then examined to identify regions of the structure that have the ability to bind to ligands. These regions are then used to identify ligands that bind to the protein.

In an aspect, a three-dimensional structural model of the protein is generated by receiving input of channel protein amino acid sequences or nucleic acid sequences encoding a channel protein into the computer system. The amino acid sequence of the channel protein is selected from the group consisting of: SEQ ID NOS:1 and 2, and conservatively modified versions thereof. The amino acid sequence represents the primary sequence of the protein, which encodes the structural information of the protein. At least 10 residues are entered into the computer system from computer keyboards or computer readable substrates that include, but are not limited to, electronic storage media (e.g., magnetic diskettes, tapes, cartridges, and chips), optical media (e.g., CD ROM), information distributed by internet sites, and RAM. The three-dimensional structural model of the channel protein is then generated by the interaction of the amino acid sequence and the computer system. The software is known to those skilled in the art.

The amino acid sequence represents a primary structure that encodes the information necessary to form the secondary, tertiary and quaternary structure of the monomer protein and channel. The software looks at certain parameters encoded by the primary sequence to generate the structural model. These parameters are referred to as energy terms,” and primarily include electrostatic potentials; hydrophobic potentials, solvent accessible surfaces, and hydrogen bonding. Secondary energy terms include vander Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Quaternary structure of multi-subunit proteins can be modeled in a similar fashion, using anisotrophy terms. These terms interface different protein subunits to energetically minimize the interaction of the subunits. In the case of channel proteins, typically four identical subunits make up the quaternary structure of the channel.

Useful Assays

Various assays are available for use in assessing the effect of a candidate molecule on functional operation of ion channels (hSlo2.2 and rSlo2). Useful nonlimiting ion channel assay methods are listed on page 432 of Drug Discovery infra. These methods includes electrophysiology (patch-clamp), binding assays, radioactive flux assays, redistribution (membrane-potential dyes), Ca+2 dyes and FRET-based voltage sensors.

Voltage Sensitive Dyes

In an aspect, a method of assaying the operation of a cloned channel biosensor involves use of voltage sensitive dyes which is disclosed in Cell-based Assays and Instrumentation for Screening Ion-channel Targets, See Drug Discovery Today, 4(9); 431-439, Gonzalez J E, Oades K, Leychkis Y, Harootunian A, Negulescu P A (1999) which is incorporated herein in its entirety by reference. This article provides a detailed analysis and operational setup techniques and considerations on pages 433 etc. There this article discloses the use of membrane based sensors based on FRET between voltage-sensing oxonol dyes and voltage-insensitive donor fluorophores associated with excitable cell membranes (plasma membranes).

In another aspect, voltage sensitive dyes which are useful in this biosensors are disclosed in Voltage-sensitive dyes for monitoring multineuronal activity in the intact central nervous system, Jian-Young W U, et al. Histochemical Journal 30, 169-187 (1998) which is incorporated herein in its entirety by reference.

In another aspect, voltage sensitive dyes which are useful in this biosensor are disclosed at http://betelgeuse.pc.cc.cmu.edu/bhe/undergradresearch/sheehan.html which is incorporated herein in its entirety by reference.

Voltage sensitive dyes are a heterogeneous group of compounds which have the capability of acting as molecular transducers which transducer changes in membrane potential into changes into flux or optical signals which are detected by capable detection assays.

Such dyes comprise slow dyes which have membrane potential response times in seconds and fast dyes which have membrane potential response times in milliseconds. Signals emanate from these dyes due to the change in potential of the dye molecules which are localized in the membrane or located very near the membrane.

In an aspect, detection methods such as direct detection methods record the potential of both the optical signal and with an electrode such as a voltage clamp at the same time if desired.

Voltage sensitive dyes are typically employed in an aqueous base composition in assays. Dissolving the voltage sensitive dyes in water takes care in that the dyes may need to be filtered to remove small crystals, however, this filtering can be done using a Millipore filter. Alternatively, warming slightly of an aqueous composition containing the dye is generally satisfactory to enhance solubilization of the dye. Hydrophobic dyes may require more extensive workup using ethanol and Pluronic F127.

In screening compounds it is highly desirable to screen candidate compounds against functional and functioning ion channels. It is important to reconstitute and measure channel function in a meaningful relevant function biological context such as in a transgenic cell so that meaningful classification of the candidate molecule as to it being an agonist, antagonist or ineffective classification is highly relevant.

In an aspect a first dye such as a coumarin is employed which is typified as a stationary donor voltage insensitive dye is used with an oxonol dye, which is an illustrative a mobile acceptor voltage sensitive dye.

This technology uses the sodium activated potassium channel proteins encoded and expressed in a transgenic cell, to screen for therapeutic compounds effective for treatment directed toward diseases and conditions affecting the heart, brain and kidney. Treatments will be directed towards protecting against the damage to cells caused by ischemia in heart and ischemia in brain during stroke or traumatic brain injury and epilepsy.

The identification of the gene encoding the sodium activated potassium channel allows for high throughput screens using cloned channels of this type. With this discovery, agonists and antagonists that are highly specific for the sodium activated potassium channel can be identified and discovered. The characterization of the basic properties of the cloned channel helps provide optimal conditions for drug screening, i.e. the best ionic conditions to maximize the sodium activated potassium channel.

As used herein, the term “agonist” refers to and includes any natural or synthetic molecule, ion or chemical moiety that is capable of opening a sodium activated potassium ion channel.

As used herein, the term “antagonist” refers to and includes any natural or synthetic molecule, ion or chemical moiety that is capable of inhibiting the action of an agonist by interacting directly or indirectly with a sodium activated potassium in channel.

In an aspect, agonists (channel openers) may be used for treatment of acute myocardial ischemia, stroke, and angina or as a prophylactic against transient ischemia due to coronary artery disease and as a preconditioning for organ transplants. Blockers may be useful for treatment of certain cardiac arrhythmias.

In an aspect, as noted above, screening technology is used to identify modulations of K_{Na}. In an aspect, a transgenic cell is prepared comprising a rSlo2 or hSlo2.2 stably integrated in its genome and subjected to one or more candidate therapeutic compounds. Assay technology such as described herein is used to detect and determine changes in the cell occurring after the addition of a candidate drug as a comparison is made with an assay profile of the transgenic cell before addition of the candidate therapeutic drug which is referred to as a baseline assay profile.

In an aspect, a comparison is performed using the array of the modulator of the cell by the added molecule and classifying the added molecule as an agonist, antagonist or ineffective depending on whether the array showed that the ion channel activity increased, decreased or did not change respectively in comparison with a baseline operation.

It is thought that molecules identified as agonists herein may be useful in the treatment of epilepsy, stroke and heart and organ transplants.

Epilepsy is understood to be a severe undesired neurological disorder which causes sudden rapid sequential bursts of electrical energy (discharges) in the brain. These sudden unexpected electrical discharges produce sudden, brief seizures. It is believed that a key to modulating epilepsy is to have the electrical membrane potential in the neuron membrane at zero or substantially zero potential so that membrane electrical activity is at zero or close to zero.

Also it is understood that sodium induced ion channels play an important role under ischemic condition in that ischemia causes an increase in sodium ion in the intracellular cell space, due primarily to the inhibition or shutdown of the normally active Na⁺/K⁺ ATPase intracellular mechanism. Without being bound by theory, it is believed that the sodium ion concentration increases in the intracellular space under ischemia insult resulting in the activation of sodium activated potassium ion channels which in turn reduces membrane electrical activity which may help protect excitable cells by conserving resources such as Na/K ATPase under ischemic conditions.

Without being bound by theory, it is believed that the cell protection brought about by ischemic conditions is that the cell electrical activity is greatly reduced.

Thus with the identification of a suitable agonist which opens a sodium activated potassium channel, it is envisioned that a cell could be provided with ischemia preconditioning to improve organ transplant, hypoxic preconditioning prior to organ transplant harvest and identify novel anticonvulsant drugs and drug therapies.

It is highly desired to identify a therapeutic candidate drug which would effectively enhance the sodium activated potassium channel thereby allowing an increased amount of potassium ions to be moved from the intracellular space to the extracellular space. The potential difference in the cell from the extracellular space to the intracellular space would be modulated in a direction of a low voltage, zero or substantially zero voltage.

Advantageously, the functional transgenic cell based assay satisfies an ever growing demand for a biosensor that identifies and categorizes candidate therapeutic drugs from among candidate drugs collections/libraries in a non-invasive assay. Candidate drugs refers to these drugs/molecules for which an identification and classification or re-classification is desired.

Once the structure has been generated, potential ligand binding regions are identified by the computer system. Three-dimensional structures for potential ligands are generated by entering amino acid or nucleotide sequences or chemical formulas of compounds, as described above. The three-dimensional structure of the potential ligand is then compared to that of the channel protein to identify ligands that bind to the channel protein. Binding affinity between the protein and ligands is determined using energy terms to determine which ligands have an enhanced probability of binding to the protein.

Computer systems are also used to screen for mutations of hSlo2.2 genes. Such mutations can be associated with disease states. Once the mutations are identified, diagnostic assays can be used to identify patients having such mutated genes associated with disease states. Identification of the mutated hSlo2.2 genes involves receiving input of a first nucleic acid sequence encoding a Na⁺ sensitive potassium channel protein having an amino acid sequence selected from the group consisting of SEQ ID NOS:1 and 2, and conservatively modified versions thereof. The sequence is entered into the computer system as described above. The first nucleic acid sequence is then compared to a second nucleic acid sequence that has substantial identity to the first nucleic acid sequence. The second nucleic acid sequence is entered into the computer system in the manner described above. Once the first and second sequences are compared, nucleotide differences between the sequences are identified. Such sequences can represent allelic differences in hSlo2.2 genes, and mutations associated with disease states.

Cellular Transfection and Gene Therapy

The present invention provides packageable hSlo2.2 channel protein nucleic acids (cDNAs), supra, for the transfection of cells in vitro and in vivo. These packageable nucleic acids can be inserted into any of a number of well known vectors for the transfection of target cells and organisms as described below. The nucleic acids are transfected into cells, ex vivo or in vivo, through the interaction of the vector and the target cell. The hSlo2.2 channel protein nucleic acid, under the control of a promoter, then expresses the Na⁺ sensitive potassium channel protein of the present invention thereby mitigating the effects of absent, partial inactivation, or abnormal expression of the hSlo2.2 channel protein gene. For example, the hSlo2.2 gene may be used to treat ischemic or digitalis toxicity conditions due to its involvement in intracellular physiology.

Such gene therapy procedures have been used to correct acquired and inherited genetic defects, cancer, and viral infection in a number of contexts. The ability to express artificial genes in humans facilitates the prevention and/or cure of many important human diseases, including many diseases that are not amenable to treatment by other therapies. As an example, in vivo expression of cholesterol-regulating genes, genes which selectively block the replication of HIV, and tumor-suppressing genes in human patients dramatically improves the treatment of heart disease, AIDS, and cancer, respectively. For a review of gene therapy procedures, see Anderson, Science (1992) 256:808-813; Nabel and Felgner (1993) TIBTECH 11: 211-217; Mitani and Caskey (1993) TIBTECH 11: 162-166; Mulligan (1993) Science 926-932; Dillon (1993) TIBTECH 11: 167-175; Miller (1992) Nature 357:455-460; Van Brunt (1988) Biotechnology 6(10):1149-1154; Vigne (1995) Restorative Neurology and Neuroscience 8:35-36; Kremer and Perricaudet (1995) British Medical Bulletin 51(1) 31-44; Haddada et al. (1995) in Current Topics in Microbiology and Immunology Doerfier and Böhm (eds) Springer-Verlag, Heidelberg Germany; and Yu et al., Gene Therapy (1994) 1:13-26.

Delivery of the gene or genetic material into the cell is the first critical step in gene therapy treatment of disease. A large number of delivery methods are well known to those of skill in the art. Such methods include, for example liposome-based gene delivery (Mannino & Gould-Fogerite, BioTechniques 6:682-691 (1988); U.S. Pat. No. 5,279,833; WO 91/06309; and Felgner et al., Proc. Natl. Acad. Sci. USA 84:7413-7414 (1987)), and replication-defective retroviral vectors harboring a therapeutic polynucleotide sequence as part of the retroviral genome (see, e.g, Miller et al., Mol. Cell. Biol. 10:4239 (1990); Kolberg, J. NIH Res. 4:43 (1992); and Cornetta et al. Hum. Gene Ther. 2:215 (1991)). Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/U594/05700.

AAV-based vectors are also used to transduce cells with target nucleic acids, e.g., in the in vitro production of nucleic acids and peptides, and in in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, Proc. Natl. Acad. Sci. USA 81:6466-6470 (1984); and Samulski et al., J. Virol. 63:03822-3828 (1989). Cell lines that can be transfected by rAAV include those described in Lebkowski et al., Mol. Cell. Biol. 8:3988-3996 (1988).

Ex Vivo Transfection of Cells

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a hSlo2.2 channel protein nucleic acid (gene or cDNA), and re-infused back into the subject organism (e.g., patient). Various cell types suitable for ex vivo transfection are well known to those of skill in the art (see, e.g., Freshney et al., Culture of Animal Cells, a Manual of Basic Technique (3d ed. 1994)).

As indicated above, in a preferred embodiment, the packageable nucleic acid which encodes a hSlo2.2 channel protein is under the control of an activated or constitutive promoter. The transfected cell(s) express a functional hSlo2.2 channel protein, which mitigates the effects of deficient or abnormal hSlo2.2 channel protein gene expression.

In Vivo Transfection

Vectors (e.g., retroviruses, adenoviruses, liposomes, etc.) containing therapeutic nucleic acids can be administered directly to the organism for transduction of cells in vivo. Administration is by any of the routes normally used for introducing a molecule into ultimate contact with blood or tissue cells. The packaged nucleic acids are administered in any suitable manner, preferably with pharmaceutically acceptable carriers. Suitable methods of administering such packaged nucleic acids are available and well known to those of skill in the art, and, although more than one route can be used to administer a particular composition, a particular route can often provide a more immediate and more effective reaction than another route.

Useful pharmaceutically acceptable carriers are determined in part by the particular composition being administered, as well as by the particular method used to administer the composition. Accordingly, there are a wide variety of suitable formulations of pharmaceutical compositions of the present invention.

The packaged nucleic acids, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Illustrative formulations suitable for parenteral administration, such as, for example, by intravenous, intramuscular, intradermal, and subceutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. The formulations of packaged nucleic acid can be presented in unit-dose or multi-dose sealed containers, such as ampules and vials.

Injection solutions and suspensions can be prepared from sterile powders, granules, and tablets of the kind previously described. Cells transduced by the packaged nucleic acid as described above in the context of ex vivo therapy can also be administered intravenously or parenterally as described above.

The dose administered to a patient, in the context of the present invention is an effective dose or should be sufficient to effect a beneficial therapeutic response in the patient over time. The dose will be determined by the efficacy of the particular vector employed and the condition of the patient, as well as the body weight or surface area of the patient to be treated. The size of the dose also will be determined by the existence, nature, and extent of any adverse side-effects that accompany the administration of a particular vector, or transduced cell type in a particular patient.

In determining the effective amount of the vector to be administered in the treatment or prophylaxis of conditions owing to diminished or aberrant expression of hSlo2.2 channel protein, an attending physician evaluates circulating plasma levels of the vector, vector toxicities, progression of the disease, and the production of anti-vector antibodies. In general, the dose equivalent of a naked nucleic acid from a vector is from about 1 μg to about 100 μg for a typical 70 kilogram patient, and doses of vectors which include a retroviral particle are calculated to yield an equivalent amount of therapeutic nucleic acid.

For administration, inhibitors and transduced cells of the present invention can be administered at an effective rate determined by the LD-50 of the inhibitor, vector, or transduced cell type, and the side-effects of the inhibitor, vector or cell type at various concentrations, as applied to the mass and overall health of the patient. Administration can be accomplished via single or divided doses.

In an aspect, transduced cells are prepared for reinfusion according to established methods (see, e.g., Abrahamsen et al., J. Clin. Apheresis 6:48-53 (1991); Carter et al., J. Clin. Apheresis, 4:113-117 (1988); Aebersold et al., J. Immunol. Meth. 112:1-7 (1988); Muul et al., J. Immunol. Methods 101:171-181 (1987); and Carter et al., Transfusion 27:362-365 (1987)). After a period of about 2-4 weeks in culture, the cells should number between 1×10⁸ and 1×10¹². In this regard, the growth characteristics of cells vary from patient to patient and from cell type to cell type. About 72 hours prior to reinfusion of the transduced cells, an aliquot is taken for analysis of phenotype, and percentage of cells expressing the therapeutic agent.

Chromosomal Assignment of the hSlo2.2 Gene

To identify the chromosomal location of hSlo2.2, the cDNA sequences encoding hSlo2.2 and the 5′ and 3′ untranslated DNA sequence, as well as DNA sequence derived from genomic DNA may be used to map the hSlo2.2 gene to a site on a number of different types of genetic maps. This may be accomplished by mapping methods which are well known in molecular genetics, including somatic cell hybrid mapping, radiation hybrid (RH) mapping, and chromosome mapping using fluorescent in situ hybridization (FISH).

An example of one of these methods which is commonly used to map a DNA sequence is the method of radiation hybrid mapping. This procedure allows one to establish with high resolution the position of a DNA sequence within the RH map by comparison of the experimental results with those obtained with known DNA markers, and evaluating the statistical probability that such a map assignment is non-random (see, e.g., Cox et al., Science 250:245-250 (1990)).

Typically, a human/hamster somatic cell hybrid panel is used for this purpose. These panels are commercially available, an example of which is the commonly used Stanford G3 panel, available from Research Genetics Inc. The panel is composed of genomic DNA from each of 83 different clonal human/hamster cell hybrid cell lines. Each cell line contains fragments of human genomic DNA in addition to the genomic host DNA of the hamster cell line from which they were derived. Since the human genomic DNA is distributed unevenly among the 83 clonal lines, PCR amplification of a specific human DNA fragment using genomic DNA from each of the clonal lines results in an amplified product in only those clonal lines containing the fragment of the corresponding human genomic DNA. Identification of the lines which produce a positive signal and which do not give a pattern that may be deconvoluted to a map position is determined by comparison of the pattern with patterns derived from other markers in a database, for example the RH server database at the Stanford Human Genome Center. Localization of the RH mapped DNA sequence to a site on a human chromosome may then be established using physical map information derived from nearby known RH markers that have already been assigned a locus on the physical map. This assignment may be accomplished using publicly available databases such as the Genome Database.

The chromosomal localization of hSlo2.2 may be used to determine whether a disease or genetic defect is attributable to changes in the genomic DNA containing the hSlo2.2 gene. Examples of such changes are well known in the literature and include point mutations, insertions, and deletions. Examples of human diseases attributable to changes in genomic DNA sequence include cystic fibrosis and long Q-T syndrome. Association of a disease with changes in the gene coding for a hSlo2.2 may be accomplished by examination of the genetics literature to find diseases for which the chromosomal assignment is already known but for which a specific mutation has not been determined. This can also be accomplished by examining genomic DNA sequence of an individual or group of individuals directly to determine if a mutation has occurred using established methods or a combination of both. Examples of such methods include but are not limited to PCR, single strand conformational polymorphism (SSCP) analysis, and direct sequencing of genomic DNA.

Alternatively, a disease may be mapped to a chromosomal location or a specific gene without prior knowledge of its identity by positional cloning or other methods know to those of skill. The identification of the gene may then be established by comparing the chromosomal location or actual DNA sequence with those derived from the literature or from databases containing known sequence data such as Genbank.

All publications and patent applications cited in this, specification are herein incorporated by reference as if each individual publication or patent application were specifically and individually indicated to be incorporated by reference.

The following examples are provided by way of illustration only and not by way of limitation. Those of skill in the art will readily recognize a variety of noncritical parameters that could be changed or modified to yield essentially similar results.

### EXAMPLES

### Example I

### Xenopus Oocyte Expression

A. Method:

To obtain efficient expression of rhSlo2.2 in Xencpus oocytes, the original rhSlo2.2 construct was cloned into pOX, a special vector optimized for oocyte expression. Capped cRNA was synthesized using the T3 mMessage mMachine kit (Ambion). rhSlo2.2 was linearized using NotI. Reactions were resuspended in nuclease-free water to a final concentration of 1.5 μg/μl. Oocytes were harvested from adult female Xenopus iaevis as described. Defolliculated oocytes were injected with approximately 75 ng of cRNA using a Drummond Nanojector. Injected oocytes were incubated at 19° C. in ND96 medium (in mM): 96 NaCl, 2 KCI, 1.8 CaCl₂, 1 MgCl₂, 5 HEPES, pH 7.5 with NaOH. Oocytes were analyzed 2-3 days after injection for single channel analysis or 3-6 days after injection for macroscopic current analysis.

In this Example, the oocyte from Zenopus laevis was used as a functioning expression system generally following the procedure reported at http://www.cx.unibe.ch/˜sigel/zenopus.html which is incorporated herein in its entirety by reference.

B. Results:

### Example II

### Electrophysiology

A. Methods:

Prior to recording, the vitelline membranes were mechanically removed. The contents of solutions used are described in the figure legends. Traces were acquired using an Axopatch 200A (Axon Instruments), digitized at 10 kHz and filtered at 2 kHz. Data were analyzed using pClamp 8.2 (Axon Instruments) and Sigmaplot 5 (Jandel Scientific) or Origin 5.0 (Microcal).

B. Results:

### Example III

### RT-PCR

A. Methods:

First strand synthesis was performed with PowerScript™ reverse transcriptase (Clonetech) for each tissue with 5.0 μg total RNA, primed with 5.0 μM random hexanucleotides, incubated with 250 μM dNTPs at 42° for 1 hour. 5% of each first strand reaction was assayed by PCR using 0.5 μM oligonucleotides, 200 μM dNTPs, and 1.0 unit of Taq DNA polymerase, cycled 35 times. Reaction products were electrophoresed on 2% agarose gels, using Tris borate (TBE) buffer and visualized with ethidium bromide. PCR primer pairs and cycling conditions available upon request.

Cell Culture

Embryonic cells were isolated and cultured as describedn Christensen et al. (Christensen et al., 2002) with the following modifications. Nematode eggs were not separated from adult carcasses in a sucrose gradient. Cellular debris and carcasses were removed upon filtration. Muscle cells were identified based on their distinctive morphology in cell culture. An integrated myo-3::GFP transformed strain, which labelled the body wall muscle cells with GFP, verified the method of identification (Christensen et al., 2002).

In Situ Muscle Recordings

Adult nematodes were filleted and prepared for single electrode whole-cell recording of body wall muscle as previously described (Richmond and Jorgensen, 1999; Wang et al., 2001).

Hypoxia Assay

Synchronous unstarved NGM agar cultures of adult C. elegans, two days post L4 stage, were transferred with 1 ml M9 to 1.5 ml polypropylene tubes and were placed in the hypoxia chamber (Form a Scientific, model #1025) filled with anoxic gas (5% CO₂, 10% H₂, 85% N₂) and maintained at 27°. The M9 was replaced 3 times with 1 ml of M9 (that had been vigorously bubbled for 30 minutes with anoxic gas) with the final wash removed to leave the animals in 1001. Oxygen tension in the hypoxia chamber was <0.2% as measured by an O₂ meter and electrode (Microelectrodes, Inc.—OM4 meter, M1730 electrode). After the hypoxic incubation period, the animals were transferred back to agar plates and allowed to recover in room air for 24 hours before scoring for spontaneous or evoked movement (touching with a platinum wire). Animals not moving were scored as dead (as a control experiment, we determined that 0% of wild-type and mutant animals (nf100) died with a similar incubation in atmospheric O₂). Students t-test was used to assess statistical significance. There was no significant change in pH in these experiments. The buffer used in the slo-2 hypoxia experiments was M9. M9 is a modified phosphate-buffered saline, 86 mM NaCI, 42 mM Na2HPO4, 22 mM KH2PO4, 1 mM MgSO4, pH=6.98-7.02. Deoxygenating M9 with the Hydrogen, Nitrogen, CO₂ mixture results in a negligible drop in pH of 0.01-0.05 units to 6.97.

In transplants, such as orthotopic cardiac transplant and orthotopic liver transplants, time is of the essence as regards retrieving a donor heart, having the donor travel to the hospital to be prepared to receive the donated organ and transporting the donated organ. It is believed that treatment of the donor tissue prior to the transplant with a suitable agonist will provide effective transplant precondition which will provide the organ recipient with a much better opportunity for a successful longer lasting transplanted organ.

It is desired to have a high transplant success rate as is the case in every operation. Several factors which have a bearing here include time limitations particularly as to recipient travel time to the transplant center following notification, recipient preparation time and time involving in transporting a donor organ to a transplant center where for implanting into the recipient. Since the total time involved is presently limited to about three hours, it would be beneficial to have that time extended so that the geographical travel rang for recipients to travel to a transplant center is increased, the time involved in prepping the recipient is expanded and the time involved for transporting the donor organ to the receipting transplant center is increased. With regard to the latter this means that the geographical range of organ donors is increased. Each and all of these go into enhancing the probably of a successful transplant to someone in need thereof.

Without being bound by theory it is believed that one can effectively identify suitable agonists which actively increase sodium activated potassium channels. Treatment of harvested tissue with these identified agonists prior to organ harvest would place the donor organ is an enhanced preconditioned pretransplant condition. Agonists which enhance (open) the sodium activated potassium ion channel would place the tissue in an electrical substitute neutral situation thus conserving precious cell resources.

Advantageously, such methods as described herein may be employed to provide ischemic precondition of a donor organ prior to transplant, provide pypoxic pre-condition prior to transplant, provide neuro-protective effect mediated by action of K9ATPase) channels, (as in an ichemic situation) and identify novel anticonvulascent drugs. Additionally, methods herein provide a method of prophylaxis treatment for ischemic conditions and transplants.

### REFERENCES

Adelman, J. P., Shen, K. Z., Kavanaugh, M. P., Warren, R. A., Wu, Y. N., Lagrutta, A., Bond, C. T., and North, R. A. (1992). Calcium-activated potassium channels expressed from cloned complementary DNAs, Neuron 9, 209-16.

Atkinson, N. S., Robertson, G. A., and Ganetzky, B. (1991). A component of calcium-activated potassium channels encoded by the Drosophila slo locus, Science 253, 55 1-5.

Bader, C. R., Bernheim, L., and Bertrand, D. (1985). Sodium-activated potassium current in cultured avian neurones, Nature 317, 540-2.

Bhattacharjee, A., Gan, L., and Kaczmarek, L. K. (2002) Localization of the Slack potassium channel in the rat central nervous system. J. Comp. Neurology 454, 24 1-254.

Bargmann, C. I. (1998). Neurobiology of the Caenorhabditis elegans genome, Science 282, 2028-33.

Bisehoff, U., Vogel, W., and Safronov, B. V. (1998). Na+-activated K+ channels in small dorsal root ganglion neurones of rat, J Physiol 510, 743-54.

Butler, A., Tsunoda, S., McCobb, D. P., Wei, A., and Salkoff, L. (1993). mSlo, a complex mouse gene encoding “maxi” calcium-activated potassium channels, Science 261, 221-4.

Christensen, M., Estevez, A., Yin, X., Fox, R., Morrison, R., McDonnell, M., Gleason, C., Miller, D. M., 3rd, and Strange, K. (2002). A primary culture system for functional analysis of C. elegans neurons and muscle cells, Neuron 33, 503-14.

Dale, N. (1993). A large, sustained Na(+)- and voltage-dependent K+ current in spinal neurons of the frog embryo, J Physiol 462, 349-72.

Dryer, S. E., Fujii, J. T., & Martin, A. R. (1989) A Na+-activated K+ current in cultured brain stem neurones from chicks. Journal of Physiology. 410:283-96,

Dryer S E. (1991) Na(±)-activated K± channels and voltage-evoked ionic currents in brain stem and parasympathetic neurones of the chick. Journal Physiol. 435:513-32,

Dryer, S. E. (1993). Properties of single Na(+)-activated K± channels in cultured central neurons of the chick embryo, Neurosci Lett 149, 133-6.

Dryer, S. E. (1994). Na(+)-activated K+ channels: a new family of large-conductance ion channels, Trends Neurosci 17, 155-60.

Dryer, S. E., Fujii, J. T., and Martin, A. R. (1989). A Na±-activated K+ current in cultured brain stem neurones from chicks, J Physiol 410, 283-96.

Egan, T. M., Dagan, D., Kupper, J., and Levitan, I. B. (1992a). Na(+)-activated K+ channels are widely distributed in rat CNS and in Xenopus oocytes, Brain Res 584, 319-21.

Egan, T. M., Dagan, D., Kupper, J., and Levitan, I. B. (1992b). Properties and rundown of sodium-activated potassium channels in rat olfactory bulb neurons, J Neurosci 12, 1964-76.

Haddad, G. G., and Jiang, C. (1993). O₂ deprivation in the central nervous system: on mechanisms of neuronal response, differential sensitivity and injury, Prog Neurobiol 40, 277-3 18.

Haimann, C., Bernheim, L., Bertrand, D., and Bader, C. R. (1990). Potassium current activated by intracellular sodium in quail trigeminal ganglion neurons, J Gen Physiol 95, 961-79.

Haimann, C., Magistretti, J., and Pozzi, B. (1992). Sodium-activated potassium current in sensory neurons: a comparison of cell-attached and cell-free single-channel activities, Pflugers Arch 422, 287-94.

Hille, B. (1992). Chapter 20: Evolution and Diversity. In Ionic Channels of Excitable Membranes (Sunderland, Sinauer Associates).

Jiang, Y., Lee, A., Chen, J., Cadene, M., Chait, B. T., and MacKinnon, R. (2002). Crystal structure and mechanism of a calcium-gated potassium channel, Nature 417, 515-22.

Jiang, Y., Pico, A., Cadene, M., Chait, B. T., and MacKinnon, R. (2001). Structure of the RCK domain from the E. coli K+ channel and demonstration of its presence in the human BK channel, Neuron 29, 593-601.

Joiner, W. J., Tang, M. D., Wang, L. Y., Dworetzky, S. I., Boissard, C. G., Gan, L., Gribkoff, V. K., and Kaczmarek, L. K. (1998). Formation of intermediate-conductance calcium-activated potassium channels by interaction of Slack and Sb subunits, Nat Neurosci 1, 462-9.

Kameyama, M. et al. Intracellular Na˜ activates a K˜ channel in mammalian cardiac cells. Nature 309, 354-6. (1984).

Kline R P, Hanna M S, Dresdner K P Jr, Wit A L. Time course of changes in intracellular K+, Na+, and pH of subendocardial Purkinje cells during the first 24 hours afier coronary occlusion. Circ Res 1992 March;70(3):566-75 Intracellular Na+ activates a K+ channel in mammalian cardiac cells, Nature 309, 354-6.

Lai, Z. F., and Nishi, K. (1998). Intracellular chloride activity increases in guinea pig ventricular muscle during simulated isehemia, Am J Physiol 275, H1613-9.

Luk, H. N., and Carmeliet, E. (1990). Na(+)-activated K+ current in cardiac cells: rectification, open probability, block and role in digitalis toxicity, Pflugers Arch 416, 766-8.

Meera, P., Wallner, M., Song, M., and Toro, L. (1997). Large conductance voltage- and calcium-dependent K⁺ channel, a distinct member of voltage-dependent ion channels with seven N-terminal transmembrane segments (S0-S6), an extracellular N terminus, and an intracellular (S9-S10) C terminus, Proc Natl Acad Sci USA 94, 14066-71.

Mistry, D. K., Tripathi, 0., and Chapman, R. A. (1997). Kinetic properties of unitary Na⁺-dependent K⁺ channels in inside-out patches from isolated guinea-pig ventricular myocytes, J Physiol 500, 39-50.

Mitani, A., and Shattock, M. J. (1992). Role of Na-activated K channel, Na—K—Cl cotransport, and Na—K pump in [K]e changes during isehemia in rat heart, Am J Physiol

Nakamura T, Hayashi H, Satoh H, Katoh H, Kaneko M, Terada H. A single cell model of myocardial reperfusion injury: changes in intracellular Na+ and Ca²⁺ concentrations in guinea pig ventricular myocytes. Mol Cell Biochem 1999 194(1-2):147-57263, H333-40.

Nicholls, J. G., Martin, A. R., and Wallace, B. G. (1992). Chapter 3: Ionic Basis of the Resting Potential. In From Neuron to Brain (Sunderland, Sinauer Associates).

Pogorelov A G, Pogorelova V N, Dubrovkin M I, Demin I P, Khrenova. Activation of specific membrane mechanisms in the myocardium cells on early stages of ischemia, Biofizika 2002 Jul-Aug;47(4):744-5 1

Richmond, J. E., and Jorgensen, E. M. (1999). One GABA and two acetylcholine receptors function at the C. elegans neuromuscular junction, Nat Neurosci 2, 791-7.

Safronov, B. V., and Vogel, W. (1996). Properties and functions of Na(+)-activated K⁺ channels in the soma of rat motoneurones, J Physiol 497, 727-34.

Schreiber, M., and Salkoff, L. (1997). A novel calcium-sensing domain in the BK channel, Biophys J 73, 1355-63.

Schreiber, M., Wei, A., Yuan, A., Gaut, J., Saito, M., and Salkoff, L. (1998). 51o3, a novel pH-sensitive K⁺ channel from mammalian spermatocytes, J Biol Chem 273, 3509-16.

Schreiber, M., Yuan, A., and Salkoff, L. (1999). Transplantable sites confer calcium sensitivity to BK channels, Nat Neurosci 2, 416-21.

Schwindt, P. C., Spain, W. J., and Crill, W. E. (1989). Long-lasting reduction of excitability by a sodium-dependent potassium current in cat neocortical neurons, J Neurophysiol 61, 233-44.

Scott, B. A., Avidan, M. S., and Crowder, C. M. (2002). Regulation of hypoxic death in C. elegans by the insulin/IGF receptor homolog DAF-2, Science 296, 2388-91.

Wallner, M., Meera, P., and Toro, L. (1996). Determinant for beta-subunit regulation in high-conductance voltage-activated and Ca(2+)-sensitive K⁺ channels: an additional transmembrane region at the N terminus, Proc Natl Acad Sci USA 93, 14922-7.

Wang, Z., Kimitsuki, T., and Noma, A. (1991). Conductance properties of the Na(±)-activated K⁺ channel in guinea-pig ventricular cells, J Physiol 433, 24 1-57.

Wang, Z. W., Saifee, 0., Nonet, M. L., and Salkoff, L. (2001). Slo-1 potassium channels control quantal content of neurotransmitter release at the C. elegans neuromuscular junction, Neuron 32, 867-81.

Wei, A., Yuan, A., Fawcett, G., Butler, A., Davis, T., Xu, S. -Y., and Salkoff, L. (2002). Efficient isolation of targeted Caenorhabditis elegans deletion strains using highly thermostable restriction endonucleases and PCR, Nucleic Acids Research 30.

Xia, X. M., Zeng, X., and Lingle, C. J. (2002). Multiple regulatory sites in large-conductance calcium-activated potassium channels, Nature 418, 880-4.

Yuan, A., Dourado, M., Butler, A., Walton, N., Wei, A., and Salkoff, L. (2000). Slo-2, a K⁺ channel with an unusual Cl⁻dependence, Nat Neurosci 3, 77 1-9.

Zhang, Z. W., Kaneda, T., Ku, K., Otaki, M., and Oku, H. (2001). Isehemic preconditioning and nicorandil pretreatment improve donor heart preservation, Jpn Circ J 65, 678-82.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of this discovery.

SEQUENCE LISTINGS:
SEQ ID 1 is
AGCGGCGCAATTAACCCTCCACTAAAGGGAACAAAAAGCTGGGTACCTAG
CTTCTTGTTCTTTTTGCAGAAGCTCAGAATAAACGCTCAACTTTGGCAGA
GCTAGCGCCACCATGAGCGACCTGGACTCCGAGGTGCTGCCCTTGCCGCC
GCGCTACCGCTTCCGGGACCTGCTGCTGGGCGACCCGTCCTTCCAGAACG
ACGACAGGGTCCAGGTGGAGTTCTACGTCAACGAGAACACCTTCAAGGAG
CGGCTCAAGCTGTTCTTCATCAAAAACCAAAGATCGAGCCTGAGGATCCG
GCTGTTCAACTTCTCCCTGAAGCTGCTCACCTGCCTGCTCTACATTGTGC
GCGTCCTGCTCGATGACCCGGCCCTGGGCATCGGATGCTGGGGCTGCCCA
AAGCAGAACTACTCCTTCAATGACTCGTCCTCCGAGATCAACTGGGCTCC
TATTCTGTGGGTGGAGAGAAAGATGACACTGTGGGCGATCCAGGTCATCG
TGGCCATAATAAGCTTCCTGGAGACGATGCTTCTCATCTACCTCAGCTAC
AAAGGCAACATCTGGGAGCAGATCTTCCGCGTGTCCTTCGTCCTGGAGAT
GATCAACACTCTGCCCTTCATCATCACGATCTTCTGGCCGCCGCTGCGGA
ACCTGTTCATCCCCGTCTTTCTGAACTGCTGGCTGGCCAAGCACGCGCTG
GAAAACATGATTAATGACTTCCACCGTGCCATCCTGCGGACACAGTCAGC
CATGTTCAACCAGGTCCTCATCCTCTTCTGCACCCTGCTGTGCCTCGTTT
TCACGGGGACCTGCGGCATCCAGCACCTGGAGCGGGCGGGCGAGAACCTG
TCCCTCCTGACCTCCTTCTACTTCTGCATCGTCACCTTCTCCACCGTGGG
CTACGGTGACGTCACGCCCAAGATCTGGCCATCGCAGCTGCTGGTGGTCA
TCATGATCTGCGTGGCCCTCGTGGTGCTCCCACTGCAGTTCGAGGAGCTC
GTCTACCTCTGGATGGAGCGGCAGAAGTCAGGGGGCAACTACAGCCGCCA
CCGTGCGCAGACGGAGAAGCACGTGGTCCTGTGTGTCAGCTCCCTCAAGA
TCGACCTTCTCATGGACTTCCTGAACGAGTTCTACGCCCACCCCCGGCTC
CAGGACTATTACGTGGTCATCCTGTGCCCCACGGAGATGGATGTCCAGGT
GCGCAGAGTCCTGCAGATCCCTCTGTGGTCCCAGCGGGTCATCTACCTCC
AGGGCTCTGCACTCAAAGACCAGCACCTCATGCGAGCCAAGATGGACAAT
GGGGAGGCCTGCTTCATCCTCAGCAGCAGGAACGAGGTGGACCGCACGGC
TGCAGACCACCAGACCATCCTGCGCGCCTGGGCCGTGAAGGACTTCGCCC
CCAACTGCCCCCTCTACGTCCAGATCCTCAAACCTGAAAACAAGTTTCAC
GTCCAAGTTTTTGCTGACCACGTGGTGTGTGAGGAGGAGTGCAAGTACGC
CATGCTGGCGCTGAACTGCATCTGCCCGGCGACCTCCACCCTCATCACCC
TGCTGGTGCACACGTCCCGCGGCCAGGAGGGACAGGAGTCTCCGGAGCAG
TGGCAGCGCATGTATGGGCGCTGCTCCGGCAACGAGGTGTACCACATCCG
CATGGGTGACAGCAAGTTCTTCCGCGAGTACGAGGGCAAGAGCTTCACCT
ACGCGGCCTTCCACGCCCACAAGAAGTATGGCGTGTGCCTCATCGGGCTG
AAGCGGGAGGACAACAAGAGCATCCTGCTGAACCCGGGGCCCCGGCACAT
CCTGGCCGCCTCTGACACCTGCTTCTACATCAACATCACCAAGGAGGAGA
ACTCGGCCTTCATCTTCAAGCAGGAGGAGAAGCGGAAGAAGAGGGCCTTC
TCGGGGCAGGGGCTGCACGAGGGTCCGGCCCGCCTGCCCGTGCACAGCAT
CATCGCCTCCATGGGGACAGTGGCCATGGACCTGCAGGGCACAGAGCACC
GGCCTACGCAGAGCGGCGGTGGGGGCGGGGGCAGCAAGCTGGCACTGCCC
ACGGAGAACGGCTCGGGCAGCCGGCGGCCCAGCATCGCGCCCGTCCTGGA
ACTGGCCGACAGCTCAGCCCTGCTGCCCTGCGACCTGCTGAGCGACCAGT
CGGAGGATGAGGTGACGCCGTCGGACGACGAGGGGCTCTCCGTGGTAGAG
TATGTGAAGGGCTACCCTCCCAACTCGCCCTACATCGGCAGCTCCCCAAC
CCTGTGCCACCTCCTGCCTGTGAAAGCCCCCTTCTGCTGCCTGCGGCTGG
ACAAGGGCTGCAAGCACAACAGCTATGAAGACGCCAAGGCCTACGGGTTC
AAGAACAAGCTGATCATCGTCTCGGCAGAGACGGCCGGCAATGGGCTGTA
CAACTTCATCGTGCCACTGCGGGCCTACTACAGATCCCGCAAGGAGCTGA
ACCCCATCGTGCTGCTGCTGGACAACAAGCCCGACCACCACTTCCTGGAA
GCCATCTGCTGCTTCCCCATGGTCTACTACATGGAGGGCTCTGTGGACAA
CCTGGACAGCCTGCTGCAGTGTGGCATCATCTATGCGGACAACCTGGTGG
TGGTGGACAAGGAGAGCACCATGAGCGCCGAGGAGGACTACATGGCGGAC
GCCAAGACCATCGTCAACGTGCAGACCATGTTCCGGCTCTTCCCCAGCCT
CAGCATCACCACGGAGCTCACCCACCCTTCCAACATGCGCTTCATGCAGT
TCCGCGCCAAGGACAGCTACTCTCTGGCTCTTTCCAAACTAGAAAAGAGG
GAGCGAGAGAATGGCTCCAACCTGGCCTTCATGTTCCGCCTGCCGTTCGC
CGCCGGCCGCGTCTTCAGCATCAGCATGTTGGACACACTGCTCTACCAGT
CCTTCGTGAAGGACTACATGATCACCATCACCCGGCTGCTGCTGGGCCTG
GACACCACGCCGGGCTCGGGGTACCTCTGTGCCATGAAAATCACCGAGGG
CGACCTGTGGATCCGCACGTACGGCCGCCTCTTCCAGAAGCTCTGCTCCT
CCAGCGCCGAGATCCCCATTGGCATCTACCGGACAGAGAGCCACGTCTTC
TCCACCTCGGAGCCCCACGACCTCAGAGCCCAGTCCCAGATCTCGGTGAA
CGTGGAGGACTGTGAGGACACACGGGAAGTGAAGGGGCCCTGGGGCTCCC
GCGCTGGCACCGGAGGCAGCTCCCAGGGCCGCCACACGGGCGGCGGTGAC
CCCGCAGAGCACCCACTGCTACGGCGCAAGAGCCTGCAGTGGGCCCGGAG
GCTGAGCCGCAAGGCGCCCAAGCAGGCAGGCCGGGCGGCGGCCGCGGAGT
GGATCAGCCAGCAGCGCCTCAGCCTGTACCGGCGCTCTGAGCGCCAGGAG
CTCTCCGAGCTGGTGAAGAACCGCATGAAGCACCTGGGGCTGCCCACCAC
CGGCTACGAGGACGTAGCAAATTTAACAGCCAGTGATGTCATGAATCGGG
TAAACCTGGGATATTTGCAAGACGAGATGAACGACCACCAGAACACCCTC
TCCTACGTCCTCATCAACCCTCCGCCCGACACGAGGCTGGAGCCCAGTGA
CATTGTCTATCTCATCCGCTCCGACCCCCTGGCTCACGTGGCCAGCAGCT
CCCAGAGCCGGAAGAGCAGCTGCAGCCACAAGCTGTCGTCCTGCAACCCC
GAGACTCGCGACGAGACACAGCTCTGAGCCAGCCCTGCACGGAGCTCAGG
CCACCAAGCCCGGGGTCCTCAGGAAGGACGTGGAGGAGCGTGTGAGGACA
CGGTGGCACTAGCGTGACCCTGGGGATGGCACACTCTACTCACCATGGCT
CCTGGGACTCCACCCTGGAAAGGAGCCCCTCATGCGGGGGGAGGGCCAGC
TCACCCCTGGGCACCTGCAGGCTAGTGAGGAGAGTTTTTTAACCTATTTT
TACACGTCGATGCAGTCCACTTCTCTTTACACAGATGTACCGCAACTCGT
GACCAGGGCTGGCTGGGAGGGCAACGCAGGGACTGGACGCCCTACAGGGC
CGAGCCCAGGCTGTGCTGGAGGGTGGGGCTGGGGTGCATGGGGAGGGGAG
CAGAACCCAGAACCCAGGAGCCCCGCGTGGGCCACACCCAACTCAGAGCC
GGCCTGAGCGTTCACGGCCAGGCAGCCTCGCTTCCTTGCAGCCAAGGGCT
GGGGGCCAGGGCTGCTGTTCTGCACTCTGGGGTGGGTGAGGGGGACCCTG
GGCTGTTTGCTGTCCCAAGCCCCTTCTGGAAGTTAGAAGCAGCAAAGGGC
CCGGGGAAGCCGGGCATGTGAGAGGGGTGCGTCCCCAGGTCCCCCAGAGG
GCCCTGTCGCCGAGGACCTTTCTGAAGGAAGCAGAAGACGCCATTTCCTC
TACTTCACACTGAACTGTCCCAGCCACTGCATCTAGGGGGCATTGGGCGG
AAGATGGTGCATTTCCATGGACCATTTTACACTTACCTTTTAAAGCAAAG
CCTCATTTTCTAAACCCCTGACTTGTGAAGCACAATTCAGCCTCCGGGCT
GGGCCACGTGGAGAGAGAGGATCTTCTCAGCAAGGCGAGATCCCGGGCGG
CGGCTGACATCAGGAGCGCCACCCTGCGTCCTTTGCTGCTGGTTCCTTAC
TGGTTTGTACGGTCAGCGCTGGAAACTTCTATTAAATGGATGCATTCTGG
AGGCATGAAGTTAAAAAAAAAAAAAAAAAAAAAAAAAAAAAAAAAAAAAA
AGGAATTCCTGCAGACCCGGGGGATCCACTAGTTNCTAGANC
And SEQ ID 2 =
ATGAGCGACCTGGACTCCGAGGTGCTGCCCTTGCCGCCGCGCTACCGCTT
CCGGGACCTGCTGCTGGGCGACCCGTCCTTCCAGAACGACGACAGGGTCC
AGGTGGAGTTCTACGTCAACGAGAACACCTTCAAGGAGCGGCTCAAGCTG
TTCTTCATCAAAAACCAAAGATCGAGCCTGAGGATCCGGCTGTTCAACTT
CTCCCTGAAGCTGCTCACCTGCCTGCTCTACATTGTGCGCGTCCTGCTCG
ATGACCCGGCCCTGGGCATCGGATGCTGGGGCTGCCCAAAGCAGAACTAC
TCCTTCAATGACTCGTCCTCCGAGATCAACTGGGCTCCTATTCTGTGGGT
GGAGAGAAAGATGACACTGTGGGCGATCCAGGTCATCGTGGCCATAATAA
GCTTCCTGGAGACGATGCTTCTCATCTACCTCAGCTACAAAGGCAACATC
TGGGAGCAGATCTTCCGCGTGTCCTTCGTCCTGGAGATGATCAACACTCT
GCCCTTCATCATCACGATCTTCTGGCCGCCGCTGCGGAACCTGTTCATCC
CCGTCTTTCTGAACTGCTGGCTGGCCAAGCACGCGCTGGAAAACATGATT
AATGACTTCCACCGTGCCATCCTGCGGACACAGTCAGCCATGTTCAACCA
GGTCCTCATCCTCTTCTGCACCCTGCTGTGCCTCGTTTTCACGGGGACCT
GCGGCATCCAGCACCTGGAGCGGGCGGGCGAGAACCTGTCCCTCCTGACC
TCCTTCTACTTCTGCATCGTCACCTTCTCCACCGTGGGCTACGGTGACGT
CACGCCCAAGATCTGGCCATCGCAGCTGCTGGTGGTCATCATGATCTGCG
TGGCCCTCGTGGTGCTCCCACTGCAGTTCGAGGAGCTCGTCTACCTCTGG
ATGGAGCGGCAGAAGTCAGGGGGCAACTACAGCCGCCACCGTGCGCAGAC
GGAGAAGCACGTGGTCCTGTGTGTCAGCTCCCTCAAGATCGACCTTCTCA
TGGACTTCCTGAACGAGTTCTACGCCCACCCCCGGCTCCAGGACTATTAC
GTGGTCATCCTGTGCCCCACGGAGATGGATGTCCAGGTGCGCAGAGTCCT
GCAGATCCCTCTGTGGTCCCAGCGGGTCATCTACCTCCAGGGCTCTGCAC
TCAAAGACCAGCACCTCATGCGAGCCAAGATGGACAATGGGGAGGCCTGC
TTCATCCTCAGCAGCAGGAACGAGGTGGACCGCACGGCTGCAGACCACCA
GACCATCCTGCGCGCCTGGGCCGTGAAGGACTTCGCCCCCAACTGCCCCC
TCTACGTCCAGATCCTCAAACCTGAAAACAAGTTTCACGTCCAAGTTTTT
GCTGACCACGTGGTGTGTGAGGAGGAGTGCAAGTACGCCATGCTGGCGCT
GAACTGCATCTGCCCGGCGACCTCCACCCTCATCACCCTGCTGGTGCACA
CGTCCCGCGGCCAGGAGGGACAGGAGTCTCCGGAGCAGTGGCAGCGCATG
TATGGGCGCTGCTCCGGCAACGAGGTGTACCACATCCGCATGGGTGACAG
CAAGTTCTTCCGCGAGTACGAGGGCAAGAGCTTCACCTACGCGGCCTTCC
ACGCCCACAAGAAGTATGGCGTGTGCCTCATCGGGCTGAAGCGGGAGGAC
AACAAGAGCATCCTGCTGAACCCGGGGCCCCGGCACATCCTGGCCGCCTC
TGACACCTGCTTCTACATCAACATCACCAAGGAGGAGAACTCGGCCTTCA
TCTTCAAGCAGGAGGAGAAGCGGAAGAAGAGGGCCTTCTCGGGGCAGGGG
CTGCACGAGGGTCCGGCCCGCCTGCCCGTGCACAGCATCATCGCCTCCAT
GGGGACAGTGGCCATGGACCTGCAGGGCACAGAGCACCGGCCTACGCAGA
GCGGCGGTGGGGGCGGGGGCAGCAAGCTGGCACTGCCCACGGAGAACGGC
TCGGGCAGCCGGCGGCCCAGCATCGCGCCCGTCCTGGAACTGGCCGACAG
CTCAGCCCTGCTGCCCTGCGACCTGCTGAGCGACCAGTCGGAGGATGAGG
TGACGCCGTCGGACGACGAGGGGCTCTCCGTGGTAGAGTATGTGAAGGGC
TACCCTCCCAACTCGCCCTACATCGGCAGCTCCCCAACCCTGTGCCACCT
CCTGCCTGTGAAAGCCCCCTTCTGCTGCCTGCGGCTGGACAAGGGCTGCA
AGCACAACAGCTATGAAGACGCCAAGGCCTACGGGTTCAAGAACAAGCTG
ATCATCGTCTCGGCAGAGACGGCCGGCAATGGGCTGTACAACTTCATCGT
GCCACTGCGGGCCTACTACAGATCCCGCAAGGAGCTGAACCCCATCGTGC
TGCTGCTGGACAACAAGCCCGACCACCACTTCCTGGAAGCCATCTGCTGC
TTCCCCATGGTCTACTACATGGAGGGCTCTGTGGACAACCTGGACAGCCT
GCTGCAGTGTGGCATCATCTATGCGGACAACCTGGTGGTGGTGGACAAGG
AGAGCACCATGAGCGCCGAGGAGGACTACATGGCGGACGCCAAGACCATC
GTCAACGTGCAGACCATGTTCCGGCTCTTCCCCAGCCTCAGCATCACCAC
GGAGCTCACCCACCCTTCCAACATGCGCTTCATGCAGTTCCGCGCCAAGG
ACAGCTACTCTCTGGCTCTTTCCAAACTAGAAAAGAGGGAGCGAGAGAAT
GGCTCCAACCTGGCCTTCATGTTCCGCCTGCCGTTCGCCGCCGGCCGCGT
CTTCAGCATCAGCATGTTGGACACACTGCTCTACCAGTCCTTCGTGAAGG
ACTACATGATCACCATCACCCGGCTGCTGCTGGGCCTGGACACCACGCCG
GGCTCGGGGTACCTCTGTGCCATGAAAATCACCGAGGGCGACCTGTGGAT
CCGCACGTACGGCCGCCTCTTCCAGAAGCTCTGCTCCTCCAGCGCCGAGA
TCCCCATTGGCATCTACCGGACAGAGAGCCACGTCTTCTCCACCTCGGAG
CCCCACGACCTCAGAGCCCAGTCCCAGATCTCGGTGAACGTGGAGGACTG
TGAGGACACACGGGAAGTGAAGGGGCCCTGGGGCTCCCGCGCTGGCACCG
GAGGCAGCTCCCAGGGCCGCCACACGGGCGGCGGTGACCCCGCAGAGCAC
CCACTGCTACGGCGCAAGAGCCTGCAGTGGGCCCGGAGGCTGAGCCGCAA
GGCGCCCAAGCAGGCAGGCCGGGCGGCGGCCGCGGAGTGGATCAGCCAGC
AGCGCCTCAGCCTGTACCGGCGCTCTGAGCGCCAGGAGCTCTCCGAGCTG
GTGAAGAACCGCATGAAGCACCTGGGGCTGCCCACCACCGGCTACGAGGA
CGTAGCAAATTTAACAGCCAGTGATGTCATGAATCGGGTAAACCTGGGAT
ATTTGCAAGACGAGATGAACGACCACCAGAACACCCTCTCCTACGTCCTC
ATCAACCCTCCGCCCGACACGAGGCTGGAGCCCAGTGACATTGTCTATCT
CATCCGCTCCGACCCCCTGGCTCACGTGGCCAGCAGCTCCCAGAGCCGGA
AGAGCAGCTGCAGCCACAAGCTGTCGTCCTGCAACCCCGAGACTCGCGAC
GAGACACAGCTCTGA
And SEQ ID 3 =
MSDLDSEVLPLPPRYRFRDLLLGDPSFQNDDRVQVEFYVNENTFKERLKL
FFIKNQRSSLRIRLFNFSLKLLTCLLYIVRVLLDDPALGIGCWGCPKQNY
SFNDSSSEINWAPILWVERKMTLWAIQVIVAIISFLETMLLIYLSYKGNI
WEQIFRVSFVLEMINTLPFIITIFWPPLRNLFIPVFLNCWLAKHALENMI
NDFHRAILRTQSAMFNQVLILFCTLLCLVFTGTCGIQHLERAGENLSLLT
SFYFCIVTFSTVGYGDVTPKIWPSQLLVVIMICVALVVLPLQFEELVYLW
MERQKSGGNYSRHRAQTEKHVVLCVSSLKIDLLMDFLNEFYAHPRLQDYY
VVILCPTEMDVQVRRVLQIPLWSQRVIYLQGSALKDQHLMRAKMDNGEAC
FILSSRNEVDRTAADHQTILRAWAVKDFAPNCPLYVQILKPENKFHVQVF
ADHVVCEEECKYAMLALNCICPATSTLITLLVHTSRGQEGQESPEQWQRM
YGRCSGNEVYHIRMGDSKFFREYEGKSFTYAAFHAHKKYGVCLIGLKRED
NKSILLNPGPRHILAASDTCFYINITKEENSAFIFKQEEKRKKRAFSGQG
LHEGPARLPVHSIIASMGTVAMDLQGTEHRPTQSGGGGGGSKLALPTENG
SGSRRPSIAPVLELADSSALLPCDLLSDQSEDEVTPSDDEGLSVVEYVKG
YPPNSPYIGSSPTLCHLLPVKAPFCCLRLDKGCKLHNSYEDAKAYGFKNK
LIIVSAETAGNGLYNFIVPLRAYYRSRKELNPIVLLLDNKPDHHFLEAIC
CFPMVYYMEGSVDNLDSLLQCGIIYADNLVVVDKESTMSAEEDYMADAKT
IVNVQTMFRLFPSLSITTELTHPSNMRFMQFRAKDSYSLALSKLEKRERE
NGSNLAFMFRLPFAAGRVFSISMLDTLLYQSFVKDYMITITRLLLGLDTT
PGSGYLCAMKITEGDLWIRTYGRLFQKLCSSSAEIPIGTYRTESHVFSTS
EPHDLRAQSQISVNVEDCEDTREVKGPWGSRAGTGGSSQGRHTGGGDPAE
HPLLRRKSLQWARRLSRKAPKQAGRAAAAEWISQQRLSLYRRSERQELSE
LVKNRMKHLGLPTTGYEDVANLTASDVMNRVNLGYLQDEMNDHQNTLSYV
LINPPDTRLEPSDIVYLIRSDPLAHVASSSQSRKSSCSHKLSSCNPETRD
ETQL
SEQ ID NO. 4:
GCGGGCGGGCGAGAACCTGTC
SEQ ID NO. 5:
GTAGAGGGGGCAGTTGGGGGCGAAGT
SEQ ID NO. 6:
ACTTCGCCCCCAACTGCCCCCTCTAC
SEQ ID NO. 7:
GTCCTCCCGCTTCAGCCCGATGAG
SEQ ID NO. 8:
GGGCTGAAGCGGGAGGACAACAAGAG
SEQ ID NO. 9:
GCTTTCACAGGCAGGAGGTGGCACAG
SEQ ID NO. 10:
ATGAGCGACCTGGACTCCGAGGTGCTG
SEQ ID NO. 11:
TCAGAGCTGTGTCTCGTCGCGAGTCTC

What is claimed is:

1. An isolated nucleic acid molecule comprising a sequence of nucleotides as shown in the at least one of SEQ ID NO:1 or SEQ ID NO:2 or the complement thereof comprises a nucleic acid sequence which encodes a polypeptide monomer having the amino acid sequence SEQ ID NO:3, which encodes a sodium activated potassium channel that is provided wherein the peptide monomer (i) has a calculated molecular weight of between about 120,000 kDa and about 150,000 kDa; (ii) has a unit conductance of about 60 to 190 pS, which depends upon the ionic conditions of measurement when the monomer is in a functional tetrameric form of a potassium channel and is expressed in a Xenopus oocyte or mammalian cell; (iii) has increased activity when intracellular sodium concentrations are raised from about 1 to about 80 mM; and (iv) specifically binds to polyclonal antibodies generated against SEQ ID NO:3, and forms a sodium activated potassium channel.

2. A nucleic acid in accordance with claim 1, wherein said nucleic acid encodes hSlo2.2.

3. A nucleic acid in accordance with claim 2 wherein the nucleic acid has a nucleotide sequence of at least one of SEQ ID NO:1 or SEQ ID NO:2.

4. A nucleic acid in accordance with claim 3 wherein the nucleic acid encodes the amino acid sequence SEQ ID NO:3.

5. A nucleic acid in accordance with claim 4 wherein the nucleic acid is amplified by primers that selectively hybridize under stringent hybridization conditions to the same sequence as the primer sets selected from the group consisting of:

| | | |
|---|---|---|
| Set 1: | | |
| Upper: | GCGGGCGGGCGAGAACCTGTC | (SEQ ID NO:4) |
| Lower: | GTAGAGGGGGCAGTTGGGGGCGAAGT | (SEQ ID NO:5) |
| Set 2: | | |
| Upper: | ACTTCGCCCCCAACTGCCCCCTCTAC | (SEQ ID NO:6) |
| Lower: | GTCCTCCCGCTTCAGCCCGATGAG | (SEQ ID NO:7) |
| Set 3: | | |
| Upper: | GGGCTGAAGCGGGAGGACAACAAGAG | (SEQ ID NO:8) |
| Lower: | GCTTTCACAGGCAGGAGGTGGCACAG | (SEQ ID NO:9) |
| Set 4: | | |
| Upper: | ATGAGCGACCTGGACTCCGAGGTGCTG | (SEQ ID NO:10) |
| Lower: | TCAGAGCTGTGTCTCGTCGCGAGTCTC | (SEQ ID NO:11) |

6. A nucleic acid in accordance with claim 5 wherein the isolated nucleic acid encodes at least 1151 contiguous amino acids forming a sodium activated potassium channel polypeptide monomer, said monomer having an amino acid sequence of SEQ ID NO:3, and conservatively modified variants thereof.

7. A nucleic acid in accordance with claim 6 wherein the nucleic acid encodes a sodium sensitive potassium channel polypeptide monomer having: (i) unit conductance of 60 pS to about 190 pS when the monomer is in a functional tetrameric form of a potassium channel and is expressed in a Xenopus oocyte or mammalian cell; (ii) a molecular weight of about 120 kDa to about 150 kDa; and (iii) increased activity above an intracellular sodium concentration of 1 mm; and
where the nucleic acid either: (i) selectively hybridizes under moderate stringency hybridization conditions to a nucleotide sequence of at least one of SEQ ID NO:1 or SEQ ID NO:2 or (ii) encodes a protein which can be encoded by a nucleic acid that selectively hybridizes under moderate stringency hybridization conditions to a nucleotide of SEQ ID NO:3.

8. An isolated nucleic acid which encodes a polypeptide monomer of a sodium activated potassium ion channel, the sequence: (i) encoding a monomer having a core domain that has greater than 60% amino acid sequence identity to amino acids 200-600 of a hSlo2.2 core domain as measured using a sequence comparison algorithm; and (ii) specifically binding to polyclonal antibodies raised against the core domain of SEQ ID NO:3.

9. An isolated polypeptide monomer of a sodium sensitive potassium channel, the monomer having: (i) a calculated molecular weight of between 120 kDa to at 150 kDa; (ii) a unit conductance of approximately 60 pS to about 190 pS when the monomer is in a functional tetrameric form of a potassium channel and is expressed in a cell; (iii) increased activity above approximately intracellular sodium concentration of 1 mm; and (iv) specifically binding to polyclonal antibodies generated against SEQ ID NO:3.

10. An antibody that selectively binds to hSlo2.2.

11. An expression vector which provides a nucleic acid encoding a polypeptide monomer of a sodium activated potassium channel, the monomer having: (i) a calculated molecular weight of about 120 kDa to abut 150 kDa; (ii) a unit conductance of about 60 pS to about 190 pS when the monomer is in a functional tetrameric form of a potassium channel and is expressed in a cell; (iii) increased activity above approximately intracellular sodium concentration of about 1 mm; and (iv) specifically binding to polyclonal antibodies generated against at least one of SEQ ID NO:1 or SEQ ID NO:2. A host cell comprising this expression vector.

12. A method for identifying a compound that modulates ion flux through a sodium activated potassium channel encoded by a gene selected from one of either hSlo2.2 or rSlo2 which comprises: (i) contacting a eukaryotic host cell or cell membrane in which has been expressed a sodium sensitive potassium channel monomer polypeptide with a candidate: (a) the peptide having a calculated molecular weight of about 120 kDa to about 150 kDa; (b) having a unit conductance of about 60 pS to about 190 pS when the monomer is in the functional tetrameric form of a potassium channel and is expressed in an eukaryotic cell; and (c) specifically binding to polyclonal antibodies generated against SEQ ID NO:3; and (ii) determining the functional effect of the compound upon the cell or cell membrane expressing the sodium activated potassium channel.

13. A method in accordance with claim 12 wherein the modulation flux of ions is determined by measuring whole cell conductance.

14. A method in accordance with claim 13 wherein the modulation is selected from increasing flux and decreasing flux.

15. A method in accordance with claim 14 wherein the sodium activated potassium channel monomer polypeptide is recombinant.

16. A method of detecting the presence of hSlo2.2 in mammalian tissue which comprises (i) isolating a functional biological sample from a patient; (ii) contacting the biological sample with a hSlo2.2 specific reagent that selectively binds to hSlo2.2 and, (iii) detecting the level of hSlo2.2 specific reagent that selectively associates with the functional biological sample.

17. A method in accordance with claim 16 wherein the hSlo2.2 specific reagent is selected from the group consisting of hSlo2.2 specific antibodies, hSlo2.2 specific oligonucleotide primers, and Slo2 nucleic acid probes.

18. A method in accordance with claim 17 wherein the biological sample is from a human.

19. A method of screening for mutations of hSlo2.2 genes, comprising (i) receiving input of a first nucleic acid sequence encoding a sodium activated potassium channel protein having a nucleotide sequence of at least one of SEQ ID NO:1 or SEQ ID NO:2, and conservatively modified versions thereof; (ii) comparing the first nucleic acid sequence with a second nucleic acid sequence having substantial identity to the first nucleic acid sequence; and (iii) identifying nucleotide differences between the first nucleic acid sequence and its second nucleic acid sequence.

20. A method for identifying a three-dimensional structure of hSlo2.2 proteins, the method comprising: (i) receiving input of about 270 to about 290 nucleotides or about 90 to about 300 amino acids of an amino acid sequence of a sodium activated potassium channel monomer or a nucleotide sequence of a gene encoding the protein, the protein having an amino acid sequence of SEQ ID NO:3, and conservatively modified versions thereof; and (ii) generating a three-dimensional structure of the protein encoded by the amino acid sequence.

21. A method in accordance with claim 20 wherein the amino acid sequence is a primary structure generated by a process comprising (i) forming a secondary structure from said primary structure using energy terms encoded by the primary structure and (ii) forming a tertiary structure from the secondary structure using energy terms encoded by the secondary structure.

22. A method in accordance with claim 21 wherein the generation includes forming a quaternary structure from the tertiary structure using anisotrophy terms encoded by the tertiary structure.

23. A method in accordance with claim 22 wherein generation further comprises identifying regions of the three-dimensional structure of the protein that bind to ligands and using the regions to identify ligands that bind to the protein.

24. A transgenic cell having a gene including isolated nucleic acids selected from one of either hSlo2.2 or rSlo2 encoding and expressing a polypeptide monomer of sodium activated potassium channels, and having an assay system operably connected thereto, wherein said cell is configurably enabled to produce an output associatively indicative of sodium activated potassium ion channel activity modulated by an externally presented molecule.

25. A biosensor comprising a transgenic cell having in its genome a gene selected from one of hSlo2.2 or rSlo2 which encodes and expresses as a protein a cloned sodium activated potassium ion channel, the transgenic cell having an assay system operatively coupled thereto wherein the assay system is configurably enabled to produce an output signal associatively indicative of sodium induced potassium ion channel modulation.

26. A method of preparing a biosensor which comprises competently and stably transfecting a gene selected from one of hSlo2.2 or rSlo2 having a nucleic acid functionally encoding and expressing a sodium activated potassium ion channel as its expression product in a cell, coupling an assay method operatively to the cell, configuring the assay method to capably produce an output associatively indicative of the activity of the channel expressed by the hSlo2.2 or rSlo2 gene in the biosensor.

27. A method for determining the expression of hSlo2.2 or rSlo2 includes using antibodies in vitro as diagnostic tools to examine hSlo2.2 or rSlo2 expression.

28. A method for treating a patient having conditions related to cardiomyocyte and neuron physiology including ischemic conditions and digitalis toxicity includes isolating cells from the patient, transfecting the isolated cells with a hSlo2.2 channel protein nucleic acid (gene or cDNA), and re-infusing the transfected cells back into the patient.

29. A method for preconditioning organ transplants includes isolating cells from the candidate organ explant, transfecting the candidate explant with a hSlo2.2 channel protein nucleic acid (gene or cDNA) and re-infusing the transfected cells back into the candidate organ explant prior to harvesting of the organ.

30. A method for preconditioning organ transplants includes transfecting the candidate explant organ with a hSlo2.2 nucleic acid (gene or cDNA).

31. A kit for determining in a biological sample the presence or absence of a normal gene selected from one of hSlow2.2 or rSlo2 or gene product encoding one of hSlo2.2 or rSlo2 encoded protein, for the presence or absence of an abnormal gene or gene product encoding a hSlow2.2 or rSlo2 sodium activated potassium channel, or quantification of the transcription levels of normal or abnormal hSlo2.2 or rSlo2 channel protein gene product, the kit comprising a stable preparation of nucleic acid probes for performing the assay of the present invention and optionally includes a hybridization solution in either dry or liquid form for the hybridization of probes to target sodium sensitive potassium channel proteins or sodium sensitive potassium channel protein nucleic acids of the present invention, a solution for washing and removing undesirable and non-hybridized nucleic acids and a substrate suitable for detecting the hybridization complex.

32. A method for modulating the membrane potential of a cell which comprises adding a voltage modulating effective amount of an agonist to a transgenic cell expressing the gene selected from one of hSlo2.2 or rSlo2 whereby the cell membrane potential is modulated to place the cell in a position of maximum potassium ion outflow. The amount of agonist is that amount which opens and renders active a sodium activated potassium ion channel yet is a nontoxic, cell life sustaining amount.

33. A method of treating a central or peripheral nervous system disorder or condition through modulation of a voltage-dependent potassium channel, includes administering to a subject in need of such treatment, an effective amount of a compound which increases the sodium activated potassium channel activity.

34. A method for reducing pain in a subject in need thereof by increasing ion flow through K potassium channels in a cell includes administering to the subject a pharmaceutical composition comprising a pharmaceutically acceptable carrier and a compound able to increase ion flow through sodium activated potassium channels, when the composition administered to the subject is a potassium channel opening amount, thereby reducing pain in the subject.


SEQUENCE LISTING (11 sequences)

SEQ ID NO: 1 - DNA, 4792 bp, Homo sapiens
  misc_feature (4785): a, t, c, g, unknown or other
agcggcgcaattaaccctccactaaagggaacaaaaagctgggtacctagcttcttgttctttttgcagaagctcagaataaacgctcaactttggcagagctagcgccaccatgagcgacctggactccgaggtgctgcccttgccgccgcgctaccgcttccgggacctgctgctgggcgacccgtccttccagaacgacgacagggtccaggtggagttctacgtcaacgagaacaccttcaaggagcggctcaagctgttcttcatcaaaaaccaaagatcgagcctgaggatccggctgttcaacttctccctgaagctgctcacctgcctgctctacattgtgcgcgtcctgctcgatgacccggccctgggcatcggatgctggggctgcccaaagcagaactactccttcaatgactcgtcctccgagatcaactgggctcctattctgtgggtggagagaaagatgacactgtgggcgatccaggtcatcgtggccataataagcttcctggagacgatgcttctcatctacctcagctacaaaggcaacatctgggagcagatcttccgcgtgtccttcgtcctggagatgatcaacactctgcccttcatcatcacgatcttctggccgccgctgcggaacctgttcatccccgtctttctgaactgctggctggccaagcacgcgctggaaaacatgattaatgacttccaccgtgccatcctgcggacacagtcagccatgttcaaccaggtcctcatcctcttctgcaccctgctgtgcctcgttttcacggggacctgcggcatccagcacctggagcgggcgggcgagaacctgtccctcctgacctccttctacttctgcatcgtcaccttctccaccgtgggctacggtgacgtcacgcccaagatctggccatcgcagctgctggtggtcatcatgatctgcgtggccctcgtggtgctcccactgcagttcgaggagctcgtctacctctggatggagcggcagaagtcagggggcaactacagccgccaccgtgcgcagacggagaagcacgtggtcctgtgtgtcagctccctcaagatcgaccttctcatggacttcctgaacgagttctacgcccacccccggctccaggactattacgtggtcatcctgtgccccacggagatggatgtccaggtgcgcagagtcctgcagatccctctgtggtcccagcgggtcatctacctccagggctctgcactcaaagaccagcacctcatgcgagccaagatggacaatggggaggcctgcttcatcctcagcagcaggaacgaggtggaccgcacggctgcagaccaccagaccatcctgcgcgcctgggccgtgaaggacttcgcccccaactgccccctctacgtccagatcctcaaacctgaaaacaagtttcacgtccaagtttttgctgaccacgtggtgtgtgaggaggagtgcaagtacgccatgctggcgctgaactgcatctgcccggcgacctccaccctcatcaccctgctggtgcacacgtcccgcggccaggagggacaggagtctccggagcagtggcagcgcatgtatgggcgctgctccggcaacgaggtgtaccacatccgcatgggtgacagcaagttcttccgcgagtacgagggcaagagcttcacctacgcggccttccacgcccacaagaagtatggcgtgtgcctcatcgggctgaagcgggaggacaacaagagcatcctgctgaacccggggccccggcacatcctggccgcctctgacacctgcttctacatcaacatcaccaaggaggagaactcggccttcatcttcaagcaggaggagaagcggaagaagagggccttctcggggcaggggctgcacgagggtccggcccgcctgcccgtgcacagcatcatcgcctccatggggacagtggccatggacctgcagggcacagagcaccggcctacgcagagcggcggtgggggcgggggcagcaagctggcactgcccacggagaacggctcgggcagccggcggcccagcatcgcgcccgtcctggaactggccgacagctcagccctgctgccctgcgacctgctgagcgaccagtcggaggatgaggtgacgccgtcggacgacgaggggctctccgtggtagagtatgtgaagggctaccctcccaactcgccctacatcggcagctccccaaccctgtgccacctcctgcctgtgaaagcccccttctgctgcctgcggctggacaagggctgcaagcacaacagctatgaagacgccaaggcctacgggttcaagaacaagctgatcatcgtctcggcagagacggccggcaatgggctgtacaacttcatcgtgccactgcgggcctactacagatcccgcaaggagctgaaccccatcgtgctgctgctggacaacaagcccgaccaccacttcctggaagccatctgctgcttccccatggtctactacatggagggctctgtggacaacctggacagcctgctgcagtgtggcatcatctatgcggacaacctggtggtggtggacaaggagagcaccatgagcgccgaggaggactacatggcggacgccaagaccatcgtcaacgtgcagaccatgttccggctcttccccagcctcagcatcaccacggagctcacccacccttccaacatgcgcttcatgcagttccgcgccaaggacagctactctctggctctttccaaactagaaaagagggagcgagagaatggctccaacctggccttcatgttccgcctgccgttcgccgccggccgcgtcttcagcatcagcatgttggacacactgctctaccagtccttcgtgaaggactacatgatcaccatcacccggctgctgctgggcctggacaccacgccgggctcggggtacctctgtgccatgaaaatcaccgagggcgacctgtggatccgcacgtacggccgcctcttccagaagctctgctcctccagcgccgagatccccattggcatctaccggacagagagccacgtcttctccacctcggagccccacgacctcagagcccagtcccagatctcggtgaacgtggaggactgtgaggacacacgggaagtgaaggggccctggggctcccgcgctggcaccggaggcagctcccagggccgccacacgggcggcggtgaccccgcagagcacccactgctacggcgcaagagcctgcagtgggcccggaggctgagccgcaaggcgcccaagcaggcaggccgggcggcggccgcggagtggatcagccagcagcgcctcagcctgtaccggcgctctgagcgccaggagctctccgagctggtgaagaaccgcatgaagcacctggggctgcccaccaccggctacgaggacgtagcaaatttaacagccagtgatgtcatgaatcgggtaaacctgggatatttgcaagacgagatgaacgaccaccagaacaccctctcctacgtcctcatcaaccctccgcccgacacgaggctggagcccagtgacattgtctatctcatccgctccgaccccctggctcacgtggccagcagctcccagagccggaagagcagctgcagccacaagctgtcgtcctgcaaccccgagactcgcgacgagacacagctctgagccagccctgcacggagctcaggccaccaagcccggggtcctcaggaaggacgtggaggagcgtgtgaggacacggtggcactagcgtgaccctggggatggcacactctactcaccatggctcctgggactccaccctggaaaggagcccctcatgcggggggagggccagctcacccctgggcacctgcaggctagtgaggagagttttttaacctatttttacacgtcgatgcagtccacttctctttacacagatgtaccgcaactcgtgaccagggctggctgggagggcaacgcagggactggacgccctacagggccgagcccaggctgtgctggagggtggggctggggtgcatggggaggggagcagaacccagaacccaggagccccgcgtgggccacacccaactcagagccggcctgagcgttcacggccaggcagcctcgcttccttgcagccaagggctgggggccagggctgctgttctgcactctggggtgggtgagggggaccctgggctgtttgctgtcccaagccccttctggaagttagaagcagcaaagggcccggggaagccgggcatgtgagaggggtgcgtccccaggtcccccagagggccctgtcgccgaggacctttctgaaggaagcagaagacgccatttcctctacttcacactgaactgtcccagccactgcatctagggggcattgggcggaagatggtgcatttccatggaccattttacacttaccttttaaagcaaagcctcattttctaaacccctgacttgtgaagcacaattcagcctccgggctgggccacgtggagagagaggatcttctcagcaaggcgagatcccgggcggcggctgacatcaggagcgccaccctgcgtcctttgctgctggttccttactggtttgtacggtcagcgctggaaacttctattaaatggatgcattctggaggcatgaagttaaaaaaaaaaaaaaaaaaaaaaaaaaaaaaaaaaaaaaaggaattcctgcagacccgggggatccactagttnctaganc

SEQ ID NO: 2 - DNA, 3615 bp, Homo sapiens
atgagcgacctggactccgaggtgctgcccttgccgccgcgctaccgcttccgggacctgctgctgggcgacccgtccttccagaacgacgacagggtccaggtggagttctacgtcaacgagaacaccttcaaggagcggctcaagctgttcttcatcaaaaaccaaagatcgagcctgaggatccggctgttcaacttctccctgaagctgctcacctgcctgctctacattgtgcgcgtcctgctcgatgacccggccctgggcatcggatgctggggctgcccaaagcagaactactccttcaatgactcgtcctccgagatcaactgggctcctattctgtgggtggagagaaagatgacactgtgggcgatccaggtcatcgtggccataataagcttcctggagacgatgcttctcatctacctcagctacaaaggcaacatctgggagcagatcttccgcgtgtccttcgtcctggagatgatcaacactctgcccttcatcatcacgatcttctggccgccgctgcggaacctgttcatccccgtctttctgaactgctggctggccaagcacgcgctggaaaacatgattaatgacttccaccgtgccatcctgcggacacagtcagccatgttcaaccaggtcctcatcctcttctgcaccctgctgtgcctcgttttcacggggacctgcggcatccagcacctggagcgggcgggcgagaacctgtccctcctgacctccttctacttctgcatcgtcaccttctccaccgtgggctacggtgacgtcacgcccaagatctggccatcgcagctgctggtggtcatcatgatctgcgtggccctcgtggtgctcccactgcagttcgaggagctcgtctacctctggatggagcggcagaagtcagggggcaactacagccgccaccgtgcgcagacggagaagcacgtggtcctgtgtgtcagctccctcaagatcgaccttctcatggacttcctgaacgagttctacgcccacccccggctccaggactattacgtggtcatcctgtgccccacggagatggatgtccaggtgcgcagagtcctgcagatccctctgtggtcccagcgggtcatctacctccagggctctgcactcaaagaccagcacctcatgcgagccaagatggacaatggggaggcctgcttcatcctcagcagcaggaacgaggtggaccgcacggctgcagaccaccagaccatcctgcgcgcctgggccgtgaaggacttcgcccccaactgccccctctacgtccagatcctcaaacctgaaaacaagtttcacgtccaagtttttgctgaccacgtggtgtgtgaggaggagtgcaagtacgccatgctggcgctgaactgcatctgcccggcgacctccaccctcatcaccctgctggtgcacacgtcccgcggccaggagggacaggagtctccggagcagtggcagcgcatgtatgggcgctgctccggcaacgaggtgtaccacatccgcatgggtgacagcaagttcttccgcgagtacgagggcaagagcttcacctacgcggccttccacgcccacaagaagtatggcgtgtgcctcatcgggctgaagcgggaggacaacaagagcatcctgctgaacccggggccccggcacatcctggccgcctctgacacctgcttctacatcaacatcaccaaggaggagaactcggccttcatcttcaagcaggaggagaagcggaagaagagggccttctcggggcaggggctgcacgagggtccggcccgcctgcccgtgcacagcatcatcgcctccatggggacagtggccatggacctgcagggcacagagcaccggcctacgcagagcggcggtgggggcgggggcagcaagctggcactgcccacggagaacggctcgggcagccggcggcccagcatcgcgcccgtcctggaactggccgacagctcagccctgctgccctgcgacctgctgagcgaccagtcggaggatgaggtgacgccgtcggacgacgaggggctctccgtggtagagtatgtgaagggctaccctcccaactcgccctacatcggcagctccccaaccctgtgccacctcctgcctgtgaaagcccccttctgctgcctgcggctggacaagggctgcaagcacaacagctatgaagacgccaaggcctacgggttcaagaacaagctgatcatcgtctcggcagagacggccggcaatgggctgtacaacttcatcgtgccactgcgggcctactacagatcccgcaaggagctgaaccccatcgtgctgctgctggacaacaagcccgaccaccacttcctggaagccatctgctgcttccccatggtctactacatggagggctctgtggacaacctggacagcctgctgcagtgtggcatcatctatgcggacaacctggtggtggtggacaaggagagcaccatgagcgccgaggaggactacatggcggacgccaagaccatcgtcaacgtgcagaccatgttccggctcttccccagcctcagcatcaccacggagctcacccacccttccaacatgcgcttcatgcagttccgcgccaaggacagctactctctggctctttccaaactagaaaagagggagcgagagaatggctccaacctggccttcatgttccgcctgccgttcgccgccggccgcgtcttcagcatcagcatgttggacacactgctctaccagtccttcgtgaaggactacatgatcaccatcacccggctgctgctgggcctggacaccacgccgggctcggggtacctctgtgccatgaaaatcaccgagggcgacctgtggatccgcacgtacggccgcctcttccagaagctctgctcctccagcgccgagatccccattggcatctaccggacagagagccacgtcttctccacctcggagccccacgacctcagagcccagtcccagatctcggtgaacgtggaggactgtgaggacacacgggaagtgaaggggccctggggctcccgcgctggcaccggaggcagctcccagggccgccacacgggcggcggtgaccccgcagagcacccactgctacggcgcaagagcctgcagtgggcccggaggctgagccgcaaggcgcccaagcaggcaggccgggcggcggccgcggagtggatcagccagcagcgcctcagcctgtaccggcgctctgagcgccaggagctctccgagctggtgaagaaccgcatgaagcacctggggctgcccaccaccggctacgaggacgtagcaaatttaacagccagtgatgtcatgaatcgggtaaacctgggatatttgcaagacgagatgaacgaccaccagaacaccctctcctacgtcctcatcaaccctccgcccgacacgaggctggagcccagtgacattgtctatctcatccgctccgaccccctggctcacgtggccagcagctcccagagccggaagagcagctgcagccacaagctgtcgtcctgcaaccccgagactcgcgacgagacacagctctga

SEQ ID NO: 3 - protein, 1204 aa, Homo sapiens
MSDLDSEVLPLPPRYRFRDLLLGDPSFQNDDRVQVEFYVNENTFKERLKLFFIKNQRSSLRIRLFNFSLKLLTCLLYIVRVLLDDPALGIGCWGCPKQNYSFNDSSSEINWAPILWVERKMTLWAIQVIVAIISFLETMLLIYLSYKGNIWEQIFRVSFVLEMINTLPFIITIFWPPLRNLFIPVFLNCWLAKHALENMINDFHRAILRTQSAMFNQVLILFCTLLCLVFTGTCGIQHLERAGENLSLLTSFYFCIVTFSTVGYGDVTPKIWPSQLLVVIMICVALVVLPLQFEELVYLWMERQKSGGNYSRHRAQTEKHVVLCVSSLKIDLLMDFLNEFYAHPRLQDYYVVILCPTEMDVQVRRVLQIPLWSQRVIYLQGSALKDQHLMRAKMDNGEACFILSSRNEVDRTAADHQTILRAWAVKDFAPNCPLYVQILKPENKFHVQVFADHVVCEEECKYAMLALNCICPATSTLITLLVHTSRGQEGQESPEQWQRMYGRCSGNEVYHIRMGDSKFFREYEGKSFTYAAFHAHKKYGVCLIGLKREDNKSILLNPGPRHILAASDTCFYINITKEENSAFIFKQEEKRKKRAFSGQGLHEGPARLPVHSIIASMGTVAMDLQGTEHRPTQSGGGGGGSKLALPTENGSGSRRPSIAPVLELADSSALLPCDLLSDQSEDEVTPSDDEGLSVVEYVKGYPPNSPYIGSSPTLCHLLPVKAPFCCLRLDKGCKHNSYEDAKAYGFKNKLIIVSAETAGNGLYNFIVPLRAYYRSRKELNPIVLLLDNKPDHHFLEAICCFPMVYYMEGSVDNLDSLLQCGIIYADNLVVVDKESTMSAEEDYMADAKTIVNVQTMFRLFPSLSITTELTHPSNMRFMQFRAKDSYSLALSKLEKRERENGSNLAFMFRLPFAAGRVFSISMLDTLLYQSFVKDYMITITRLLLGLDTTPGSGYLCAMKITEGDLWIRTYGRLFQKLCSSSAEIPIGIYRTESHVFSTSEPHDLRAQSQISVNVEDCEDTREVKGPWGSRAGTGGSSQGRHTGGGDPAEHPLLRRKSLQWARRLSRKAPKQAGRAAAAEWISQQRLSLYRRSERQELSELVKNRMKHLGLPTTGYEDVANLTASDVMNRVNLGYLQDEMNDHQNTLSYVLINPPPDTRLEPSDIVYLIRSDPLAHVASSSQSRKSSCSHKLSSCNPETRDETQL

SEQ ID NO: 4 - DNA, 21 bp, Artificial Sequence
  Description of Artificial Sequence Primer
gcgggcgggcgagaacctgtc

SEQ ID NO: 5 - DNA, 26 bp, Artificial Sequence
  Description of Artificial Sequence Primer
gtagagggggcagttgggggcgaagt

SEQ ID NO: 6 - DNA, 26 bp, Artificial Sequence
  Description of Artificial Sequence Primer
acttcgcccccaactgccccctctac

SEQ ID NO: 7 - DNA, 24 bp, Artificial Sequence
  Description of Artificial Sequence Primer
gtcctcccgcttcagcccgatgag

SEQ ID NO: 8 - DNA, 26 bp, Artificial Sequence
  Description of Artificial Sequence Primer
gggctgaagcgggaggacaacaagag

SEQ ID NO: 9 - DNA, 26 bp, Artificial Sequence
  Description of Artificial Sequence Primer
gctttcacaggcaggaggtggcacag

SEQ ID NO: 10 - DNA, 27 bp, Artificial Sequence
  Description of Artificial Sequence Primer
atgagcgacctggactccgaggtgctg

SEQ ID NO: 11 - DNA, 27 bp, Artificial Sequence
  Description of Artificial Sequence Primer
tcagagctgtgtctcgtcgcgagtctc